(19) United States
(12) Patent Application Publication
(10) Pub. No.: US 2003/0220524 A1
(43) Pub. Date: Nov. 27, 2003

(54) Sulfur containing compounds

(76) Inventors: Richard Francis Langler, New Brunswick (CA); Felix J Baerlocher, New Brunswick (CA); Linda Z Penn, Toronto (CA)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/204,944

(22) PCT Filed: Feb. 26, 2001
(86) PCT No.: PCT/CA01/00234

(30) Foreign Application Priority Data
Feb. 25, 2000 (CA) 2299247

(51) Int. Cl. C07C 323/65

(52) U.S. Cl. 568/23; 558/250

(57) ABSTRACT

This invention is directed to novel and known stufur containing compounds and pharmaceutically acceptable salts thereof that have utility as antifungals and as antiproliferative agents against mammalian cells, in particular cancer cells and most particularly leukemia-derived cells. The invention provides a method for synthesizing certain of the sulfur containing compounds that is more efficient than previously known methods.

### RELATED APPLICATIONS

This application claims the benefit of the priority of the filing dates of U.S. Provisional Patent Application No. 60/185,189, filed Feb. 25, 2000 and Canadian Patent Application No. 2,299,247, filed Feb. 25, 2000.

### FIELD OF THE INVENTION

This invention is directed to novel and known sulfur containing compounds and pharmaceutically acceptable salts thereof that have utility as antifungal agents and as antiproliferative agents against mammalian cells, in particular cancer cells and most particularly leukemia-derived cells. The invention provides a method for synthesizing certain of the sulfur containing compounds that is more efficient than previously known methods.

### BACKGROUND OF THE INVENTION

There is an enormous need worldwide for novel, safe, effective therapeutics in the clinical treatment of cancers. The majority of chemotherapeutics presently available are less than ideal as they show non-specific, genotoxic killing of both normal as well as tumour cells. Recent success stories suggest natural products research will uncover new molecules to help fight the cancer problem (Cardenas, M. E., Sanfridson, A., Cutler, N. S., and Heitman, J. (1998) Signal-transduction cascades as targets for therapeutic intervention by natural products. Trends Biotechnol.16:427-33, Marks, P. A., Richon, V. M. and Rifkind, R. A. (2000) Histone deacetylase inhibitors: inducers of differentiation or apoptosis of transformed cells. J Natl Cancer Inst. 92::1210-6). For example, the taxanes (taxol), derived from the bark of the yew tree, have emerged as effective anti-tumour agents in a wide variety of malignancies (Vaishampayan, U., Parchment, R. E., Jasti, B. R., and Hussain, M. (1999) Taxanes: an overview of the pharmacokinetics and pharmacodynamics. i Urology 54, 22-9, Walsh, V., and Goodman, J. (1999) Cancer chemotherapy, biodiversity, public and private property: the case of the anti-cancer drug taxol. Soc Sci Med 49, 1215-25). Enormous experimental effort has led to the identification of organosulfur compound (OSCs) as the active components of medicial plants such as garlic, onions, the leaves of the Mahogany tree, yet this knowledge is primarily applied to nutritional aspects of cancer prevention strategies and not directly to acute treatment protocols (Fukushima, S., Takada, N., Hori, T., and Wanibuchi, H. (1997) Cancer prevention by organosulfur compounds from garlic and onion. J Cell Biochem Suppl 27, 100-5, Jiao, D., Smith, T. J., Yang, C. S., Pittman, B., Desai, D., Amin, S., and Chung, F. L. (1997) Chemopreventive activity of thiol conjugates of isothiocyanates for lung tumorigenesis. Carcinogenesis 18, 2143-7, Reddy, B. S., Rao, C. V., Rivenson, A., and Kelloff, G. (1993) Chemoprevention of colon carcinogenesis by organosulfur compounds. Cancer Res 53, 3493-8, Wargovich, M. J. (1987) Diallyl sulphide, a flavor component of garlic (Allium sativum), inhibits dimethylhydrazine-induce colon cancer. Carcinogenesis 8, 487-9, Wargovich, M. J., Imada, O., and Stephens, L. C. (1992) Initiation and post-initiation chemopreventive effects of diallyl sulphide in esophageal carcinogenesis. Cancer Lett 64, 39-42, Wargovich, M. J., Woods, C., Eng. V. W., Stephens, L. C. and Gray, K. (1988) Chemoprevention of N-nitrosomethylbenzylamine-induced esophageal cancer in rats by the naturally occurring thioether, diallyl sulphide. Cancer Res 48, 6872-5). It appears that the knowledge gained from natural products research is not widely and immediately exploited for the treatment of cancer primarily because of technical limitations. For instance, only a very low yield of active metabolite can be recovered from typical natural sources, the extraction procedure often leads to mixtures of structural relatives of varying specific activity compared to the target natural product, there can be environmental issues associated with harvesting medicinal plants, and mining the active component is often labour-intensive, difficult to quality assure and is expensive to produce in large quantities. Numerous studies have shown that OSCs have antiproliferative potential (Jogia, M K, Andersen, R J, Mantus, E K and Clardy, J (1989) Dysoxysulfone, a sulfur rich metabolite from the Fijian medicinal plant Dysoxylum Richii. Tetrahedron Letters 30: 4919-4920, Block, E, DeOrazio, R and Thiruvazhi, M (1994) Simple total synthesis of biologically-active pentathiadecane natural-products, 2,4,5,7,9-pentathiadecane 2,2,9,9-tetraoxide (dysoxysulfone), from Dysoxylum-Richii, and 2,3,5,7,9-pentathiadecane 9,9-dioxide, the misidentified lenthionine precursor Se-3 from Shiitake mushroom (Lentinus-Edodes). J Org Chem 59: 2273-2275, Perchellet, J P, Perchellet, E M and Belman, S (1990) Inhibition of DMBA-induced mouse skin tumorigenesis by garlic oil and inhibition of two tumor-promotion stages by garlic and onion oils. Nutr Cancer 14: 183-93). Despite the documented biological activity of OSC (Block, E: The organosulfur chemistry of the genus Allium—implications for the organic chemistry of sulfur. Angew. Chem. Int. Ed. Engl. 31: 1135-1178, 1992, Lea, M A: Organosulfur compounds and cancer. Adv Exp Med Biol 401: 147-54, 1996), the key structural features that direcuy contribute to their antiproliferative activity remains unclear.

There is also a need for effective antifungal agents that are readily biodegradable and which can be used against a wide variety of pathogens in both animals and humans.

There are known sulfur containing compounds which have been found to be useful as antifungal agents. For example, U.S. Pat. No. 5,648,354 issued Jul. 15, 1997 to Bierer, et al and U.S. Pat. No. 5,580,897 issued Dec. 3, 1996 to Bierer, et al and U.S. Pat. No. 5,583,235 issued Dec. 10, 1996 to Bierer, et al disclose novel 1,2-dithiin compounds having such utility.

In U.S. Pat. No. 5,698,564 issued Dec. 16, 1997 to Katsuyama, et al there are described diphenyl disulphide compounds having an inhibiting activity against the production of Interleukin-1β (IL-1β) or the release of Tumor Necrosis Factor α (TNFα), which are useful in the treatment or prophylaxis of diseases such as chronic rheumatism and sepsis.

The literature contains a number of papers disclosing antifungal compounds of the type described herein. Baerlocher, Felix Jacob, et al Aust. J. Chem., 1999, 52, 167-172 entitled Structure-Activity Relationship for Selected Sulfur-Rich Antifungal Compounds reported that the inhibition of fungal growth correlates with the presence of both sulfone and disulphide functional properties. The disclosures of this paper are incorporated herein by reference.

In Langler, Richard Francis, et al Aust. J. Chem published Febery 2000, and entitled A New Synthesis for Antifungal α-Sulfone Disulphides, there is described the preparation of several new α-sulfone disulphides using an α-ester disulphide precursor. These α-sulfone disulphides were all shown to be fungitoxic against Aspergillis niger and Aspergillis flavus. The disclosures of this paper are incorporated herein by reference.

In 1989, Andersen et al. reported the isolation and structure proof of dysoxysulfone (CH₃SO₂CH₂SCH₂SSCH₂SO₂CH₃) (see Tetra. Lett. 30, 4919 (1989)). Sample size was limited and the compound showed some antibiotic activity.

In 1989, Block et al. described a synthesis for dysoxysulfone, which provided larger amounts (see Block et al., J. Org. Chem. 59, 2273 (1994)). They showed that dysoxysulfone and some related natural products were active against Candida albicans, a P388 murine leukemia cell line, Staphylococcus aureus, Bacillus subtilis, a human adrenocarcinoma cell line and a human ovarian carcinoma cell line.

U.S. Pat. No. 4,643,994 granted to Block et al. described sulfur compounds like 3 as antithrombotic agents.

RSO₂CH₂CH═CHSSR′3

The patent describes the use of these compounds against “bacteria and fungi”, as well as for “flavor enhancers in foods”. In Block's patent, compounds like 2 of the formula RSO₂CH₂SSR′ were inadvertently included. In 1987, Block et al. obtained a Certificate of Correction, which withdrew compounds like 2 from the patent.

The following publications are relevant to the present invention:

Langler, Richard Francis, et al.: A New Synthesis for Antifungal α-Sulfone Disulphides. Aust J. Chem., 1999, 52, 1119-1121.

Wong, W. Wei-Lynn, et al.: Novel Synthetic Organosulfur Compounds Induce Apoptosis of Human Leukemic Cells. Anticancer Research 20: 1367-1374 (2000).

Baerlocher, Felix Jakob, et al.: Structure-Activity Relationships for Selected Sulfur-Rich Antifungal Compounds. Aust. J. Chem., 1999, 52,167-172.

Baerlocher, Felix Jakob, et al.: Antifungal Thiosulfonates: Potency with Some Selectivity. Aust. J. Chem., 2000, 53, 399-402.

Baerlocher, Felix Jakob, et al.: New and More Potent Antifungal Disulphides. Aust J. Chem., 2000, 53, 1-5.

“New Antifungal Disulphides: Approaching Submicrogram Toxicity”, F. J. Baerlocher,, M. O. Baerlocher, C. L. Chaulk, R. F. Langler and E. M. O'Brien, Sulfur Lett.,—in press

The disclosures of theseferences are incorporated herein by reference.

### SUMMARY OF THE INVENTION

In one aspect of the invention, there are provided novel sulfone disulphides of the general formula

RSO₂CH₂SSR¹ I

wherein R is phenyl or lower alkyl, and R¹ is lower alkyl or phenyl.

This invention provides specific novel sulfone disulphides of this general formula selected from the group consisting of C₆H₅SO₂CH₂SSCH₃, CH₃SO₂CH₂SSCH₃, and CH₃SO₂CH₂SSC₆H5.

Another aspect of the invention provides a process for preparing a sulfone disulphide of the general formula I as described above, which comprises reacting a transition metal oxidant or a peroxyanhydride oxidant with a symmetrical dialkyl or arylalkyl disulphide to obtain an α-ester disulphide compound of the formula

RSSCH₂OC(O)R¹

wherein R and R¹ are as defined above, which compound is further reacted with a sulfinic acid salt to obtain the title compound.

The invention also provides compounds of the general formula RSSCH₂OC(O)R¹, wherein R and R¹ may be the same or different and each is selected from the group of substituents comprising lower alkyl or phenyl. Preferably, lower alkyl is methyl or ethyl.

The invention also provides a process for making the compounds of the formula defined above, which comprises reacting a transition metal oxidant or a peroxyanhydride oxidant with a symmetrical dialkyl or arylalkyl disulfide to obtain the α-ester disulfide compound.

In yet another aspect of the invention, there is provided a process for preparing a compound of the formula PhSO₂CH₂SSCH₃, wherein Ph is phenyl which comprises reacting a transition metal oxidant with dimethyl disulphide to obtain an α-ester disuiphide compound of the formula CH₃SSCH₂OC(O)CH₂CH₃, which compound is further reacted with the sodium salt of p-toluenesulfinic acid in either aqueous acetonitrile or aqueous acetone to obtain the title compound or the compound is further reacted with potassium p-toluenesulfonate to yield the title compound.

Another aspect of the invention provides a process for preparing compounds of the formula RSO₂CH₂SSR¹, wherein R and R¹ may be the same or different and each is selected from lower alkyl and phenyl, which comprises reacting a transition metal oxidant or a peroxyanhydride oxidant with a symmetrical dialkyl or arylalkyl disulfide to obtain the α-ester disulfide compound RSSCH₂OC(O)R¹, wherein R and R¹ are as defined above, which compound is further reacted with a sulfinic acid salt to obtain the required compound.

Another novel compound is of the formula ClSCH₂OC(O)CH₂CH₃, which is useful as an intermediate.

Another part of the invention comprises antifungal agents comprising as active ingredients a therapeutically effective amount of at least one compound of the formula

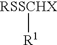

wherein R¹ is H or CH₃;

R is CH₃, CH₂CH₃, C₆H₅, o-CH₃O₂C(C₆H₄), o-CH₃SO₂(C₆H₄), p-CH₃SO₂(C₆H₄), o-NO₂(C₆H₄), m-NO₂(C₆H₄), p-NO₂(C₆H₄), or CH₂O₂CCH₂CH₃; and

X is SO₂(C₆H₄)CH₃-p, SO₂CH₃, SO₂C₆H₅, SO₂CH₂CH₃, H, O₂CCH₃, O₂CCH₂CH₃, or CO₂CH₃;

and a pharmaceutically acceptable carrier.

Another aspect of the invention provides antifungal agents comprising as active ingredient at least one antifungally active compound of the formulae

RSO₂CH₂SSR¹

wherein R is lower alkyl or phenyl and R¹ is lower alkyl or phenyl, and

RC(O)OCH₂SSR¹

wherein R and R¹ are as defined above optionally together with pharmaceutically acceptable carriers.

In yet another aspect of the invention, there is provided antifungal agents as described above wherein the active ingredient is selected from the group of compounds consisting of

o-CH₃OC(O)(C₆H₄)SSCH₃,

[o-CH₃SO₂(C₆H₄)S]₂,

[p-CH₃SO₂(C₆H₄)S]₂,

m-O₂N(C₆H₄)SSCH₃,

o-O₂N(C₆H₄)SSCH₃,

p-O₂N(C₆H₄)SSCH₃,

p-CH₃(C₆H₄)SO₂CH₂SSCH₃,

C₆H₅SO₂CH₂SSCH₃,

CH₃SO₂CH₂SSCH₃,

CH₃CH₂C(O)OCH₂SSCH₃,

CH₃SO₂CH₂SSPh,

CH₃SO₂CH₂SSCH₂CH₃,

CH₃SSCH₂OC(O)CH₃,

CH₃SSCH₂OC(O)CH₂CH₃,

CH₃SSCH₂OC(O)Ph, and

PhSSCH₂OC(O)CH₂CH₃,

optionally together with a pharmaceutically acceptable carrier.

Another aspect of the invention provides an antiproliferative agent have against mammalian cells comprising as active ingredient at least one compound of the formulae

RSO₂CH₂SSR¹

wherein R is lower alkyl or phenyl and R¹ is lower alkyl or phenyl, and

RC(O)OCH₂SSR¹

wherein R and R¹ are as defined above, and excluding those compounds disclosed herein that do not exhibit such activity, and optionally together with conventional pharmaceutically acceptable ingredients.

Another aspect of the invention provides an antiproliferative agent active against mammalian cells comprising as active ingredient at least one compound selected from the group of compounds consisting of

p-CH₃(C₆H₄)SO₂CH₂SSCH₃,

PhSSPh,

CH₃SO₂CH₂SSPh,

CH₃SO₂CH₂CH₂SSCH₃,

CH₃SSCH₂C(O)OCH3, and

CH₃SSCH₂OC(O)CH₃, and optionally together with conventional pharmaceutically acceptable ingredients.

In yet another aspect of the invention, there is provided the use of at least one compound of the formulae

RSO₂CH₂SSR¹ (I)

wherein R is lower alkyl or phenyl and R¹ is lower alkyl or phenyl, and

RC(O)OCH₂SSR¹ (II)

wherein R and R¹ are as defined above, and excluding those compounds disclosed herein that do not exhibit such activity in the preparation of an antifungal agent for the treatment of micoses.

Another aspect of the invention provides the use as described above wherein the agent is for the treatment of aspergillosis.

The invention also provides the use of at least one compound of the formulae

RSO₂CH₂SSR¹ (I)

wherein R is lower alkyl or phenyl and R¹ is lower alkyl or phenyl, and

RC(O)OCH₂SSR¹ (II)

wherein R and R¹ are as defined above, and excluding those compounds disclosed herein that do not exhibit such activity, in the preparation of an antiproliferative agent active against mammalian cells for the treatment of cancer.

In yet another aspect of the invention, there is provided the use of at least one compound selected from the group of compounds as described above in the preparation of a medicament for the treatment of cancer.

### DETAILED DESCRIPTION OF THE INVENTION

The following correlates the chemical structures and the chemical names of the compounds discussed in this application.

| Chemical Structure | Chemical Name |
|---|---|
| CH₃SO₂CH₃ | dimethyl sulfone |
| CH₃SSCH₃ | dimethyl disulphide |
| CH₃SCH₂SCH₂SCH₃ | 2,4,6-trithiaheptane |
| CH₃SO₂CH₂SCH₃ | 2,4-dithiapentane 2,2-dioxide |
| CH₃SO₂CH₂SCH₂SO₂CH₃ | 2,4,6-trithiaheptane 2,2,6,6-tetraoxide |
| CH₃SCH₂SSCH₃ | 2,3,5-trithiahexane |
| CH₃SO₂CH₂SSPh | 5-phenyl-2,4,5-trithiapentane 2,2-dioxide |
| CH₃SO₂CH₂SSCH₂CH₃ | 2,4,5-trithiaheptane 2,2,-dioxide |
| CH₃SO₂CH₂CH₂SSCH₃ | 2,3,6-trithiaheptane 6,6,-dioxide |
| CH₃SO₂CH₂CH₂CH₂SSCH₃ | 2,3,7-trithiaoctane 7,7-dioxide |
| CH₃SSCH₂C(O)OCH₃ | methyl 3,4-dithiopentanoate |
| CH₃SSCH₂OC(O)CH₃ | 2,3-dithiabutyl acetate |
| CH₃SSCH₂CH₂OC(O)CH₃ | 3,4-dithiapentyl acetate |
| p-CH₃(C₆H₄)SO₂CH₂SSCH₃ | α-p-toluenesulfonyl dimethyl disulphide |
| C₆H₅SO₂CH₂SSCH₃ | α-phenylsulfonyl dimethyl disulphide |
| CH₃SO₂CH₂SSCH₃ | α-methylsulfonyl dimethyl disulphide |
| p-CH₃(C₆H₄)SO₂CH₂SH | α-p-toluenesulfonyl methyl mercaptan |
| CH₃CH₂C(O)OCH₂SSCH₃ | 2,3-dithiabutyl propionate |
| C₆H₅SSSCH₃ | phenyl methyl disulphide |
| C₆H₅SSSPh | diphenyl disulphide |
| o-CH₃OC(O)(C₆H₄)SSCH₃ | methyl-o(methyldithio)benzoate |
| [o-CH₃SO₂(C₆H₄)S]₂ | o-methylsulfonylphenyl disulphide |
| [p-CH₃SO₂(C₆H₄)S]₂ | p-methylsulfonylphenyl disulphide |
| m-O₂N(C₆H₄)SSCH₃ | methyl m-nitrophenyl disulphide |
| o-O₂N(C₆H₄)SSCH₃ | methyl o-nitrophenyl disulphide |
| p-O₂N(C₆H₄)SSCH₃ | methyl p-nitrophenyl disulphide |
| p-CH₃SO₂(C₆H₄)OSO₂CH₃ | p-methylsulfonylphenyl methanesulfonate |
| p-O₂N(C₆H₄)OSO₂CH₃ | p-nitrophenyl methanesulfonate |
| CH₃SSCH₂OC(O)CH₃ | 2,3-dithiabutyl acetate |
| CH₃SSCH₂OC(O)CH₂CH₃ | 2,3-dithiabutyl propionate |
| CH₃SSCH₂OC(O)Ph | 2,3-dithiabutyl benzoate |
| PhSSCH₂OC(O)CH₂CH₃ | 1-phenyl-1,2-dithiapropyl propionate |
| (CH₃OC(O)CH₂S)₂ | dimethyl 3,4-dithiaadipate |
| CH₃OC(O)CH₂SSCH₂OC(O)C₂H₅ | methyl 3,4-dithia-5-propionoxypentanoate |
| (C₂H₅C(O)OCH₂S)₂ | 2,3-dithiabutane-1,4-dipropionate |
| C₂H₅OC(O)CH₂SSCH₂SO₂(C₆H₄)-p-CH₃ | 1-p-toluenesulfonyl-4-propionoxy-2,3-dithiabutane |
| (C₆H₅S)C(O)CH₂SSCH₃ | phenacyl methyl disulphide |

Biological Testing

Anti-fungal Activity

Sulfur compounds were tested for antifungal activity against pure cultures of Aspergillus niger and Aspergillus flavus supplied by Ward's Natural Science Ltd (St. Catharines, Ontario, Canada). They were maintained on Sabouraud Dextrose Agar.

For the test, 6 agar plugs (5 mm diameter) were cut from a 5-8 day old colony and homogenized in distilled, sterilized water (2 ml). A portion of this suspension (0.5 ml) was transferred aseptically to a Petri plate with Sabouraud Dextrose Agar (15 ml) and spread evenly over the entire surface. Each plate was supplied with four evenly spaced paper disks (7 mm, Whatman Number 1 filter paper) containing the test compound (0, 25, 50 and 100 μg respectively). Each test compound was applied to the disks as a solution (50 mg compound/10 ml acetone). Control disks were treated with neat acetone (20 μl). Test plates with fungal homogenates and disks were incubated at 18-20° C. for 2 days. For each test four replicate plates were used. The diameter of the clear zone surrounding the disk was taken as the indicator of antifungal activity.

Reasonable levels of antifungal activity can be gauged from the result of (CH₃SCH₂S)₂ which is a known antifungal natural product which shows clear zone diameters of 2.5 and 2.3 mm against A. niger and A. flavus, respectively, at a dose of 100 μg/disk.

TABLE 3

| | | | Diameter (mm) | |
|---|---|---|---|---|
| | Cpd. | Disk | of clear Zone | |
| Compound | No. | (μg/disk) | A. niger | A. flavus |
| CH₃SO₂CH₃ | (2) | 100 | 0 | 0 |
| CH₃SSCH₃ | (3) | 100 | 0 | 0 |
| CH₃SCH₂SCH₂SCH₃ | (4) | 100 | 0 | 0 |
| CH₃SO₂CH₂SCH₃ | (5) | 100 | 0 | 0 |
| CH₃SO₂CH₂SCH₂SO₂CH₃ | (6) | 100 | 0 | 0 |
| CH₃SCH₂SSCH₃ | (7) | 100 | 0 | 0 |
| CH₃SO₂CH₂SSPh | (8) | 25 | 0.8 | 1.8 |
| CH₃SO₂CH₂SSCH₂CH₃ | (9) | 25 | 4.8 | 3.3 |
| CH₃SO₂CH₂CH₂SSCH₃ | (10) | 100 | 0 | 0 |
| CH₃SO₂CH₂CH₂CH₂SSCH₃ | (11) | 100 | 0 | 0 |

TABLE 3

| | | | Diameter (mm) of | |
|---|---|---|---|---|
| | | Disk | clear Zone | |
| Compound | Cpd. No. | (μg/disk) | A. niger | A. flavus |
| CH₃SSCH₂C(O)OCH₃ | (12) | 100 | 0 | 0 |
| CH₃SSCH₂OC(O)CH₃ | (13) | 100 | 2.3 | 2.3 |
| CH₃SSCH₂CH₂OC(O)CH₃ | (14) | 100 | 0 | 0 |

Compounds (8) and (9) show activity against Aspergillus niger and Aspergillus flavus comparable in magnitude to that reported for Dysoxysulfone (1) against Staphylococcus aureus, Bacillus subtilis and Candida albicans (Block, E., DeOrazio, R., and Thiruvazhi, M., J. Org. Chem., 1994, 59, 2273). It would appear that the inhibition of fungal growth correlates with the presence of both sulfone and disulphide functional groups.

TABLE 4

| | | | Diameter (mm) | |
|---|---|---|---|---|
| | Cpd. | Disk | of clear Zone | |
| Compound | No. | (μg/disk) | A. niger | A. flavus |
| p-CH₃(C₆H₄)SO₂CH₂SSCH₃ | (4) | 25 | 10.9 | 8.0 |
| C₆H₅SO₂CH₂SSCH₃ | | 25 | 5.5 | 4.2 |
| CH₃SO₂CH₂SSCH₃ | | 25 | 2.7 | 3.8 |
| p-CH₃(C₆H₄)SO₂CH₂SH | | 100 | 0 | 0 |
| CH₃CH₂C(O)OCH₂SSCH₃ | (2) | 25 | 14.8 | 7.6 |

Qualitatively, the observed toxicity of the compounds in Table 4 is in complete accord with the earlier proposal (Baerlocher, F. J., Langler, R. F., Frederiksen, M. U., Georges, N. M., and Witherell, R. D., Aust. J. Chem, 1999, 52, 167) that activated antifungal disulphides (those with a reasonable leaving group attached to the α-carbon) will have pronounced antifungal activity. Quantitatively, the first and last compounds in Table 4 are the most potent fungitoxic disuiphides described to date.

TABLE 5

| | |
|---|---|
| | Diameter (mm) of |
| Disk | clear Zone |

| Compound | Cpd. No. | (μg/disk) | A. niger | A. flavus |
|---|---|---|---|---|
| C₆H₅SSCH₃ | (4) | 100 | 0 | 0 |
| C₆H₅SSPh | (5) | 100 | 0 | 0 |

TABLE 5

| | | | Diameter (mm) of | |
|---|---|---|---|---|
| | | Disk | clear Zone | |
| Compound | Cpd. No. | (μg/disk) | A. niger | A. flavus |
| o-CH₃OC(O)(C₆H₄)SSCH₃ | (6) | 25 | 2.8 | 4.3 |
| [o-CH₃SO₂(C₆H₄)S]₂ | (7) | 25 | 3.3 | 3.0 |
| [p- CH₃SO₂(C₆H₄)S]₂ | (8) | 25 | 4.0 | 6.9 |
| m-O₂N(C₆H₄)SSCH₃ | (9) | 10 | 1.7 | 1.8 |
| o-O₂N(C₆H₄)SSCH₃ | (10) | 10 | 2.8 | 1.9 |
| p-O₂N(C₆H₄)SSCH₃ | (11) | 10 | 3.9 | 4.0 |
| p-CH₃SO₂(C₆H₄)OSO₂CH₃ | (12) | 100 | 0 | 0 |
| p-O₂N(C₆H₄)OSO₂CH₃ | (13) | 100 | 0 | 0 |

Toxicity testing against A. niger and A. flavus revealed that (5) is considerably more potent than any other compound disclosed herein. The simple disulphides (4) and (5) are completely inactive against A. niger and A. flavus at a dose of 100 μg/disk.

TABLE 6

| | | | Diameter (mm) of | |
|---|---|---|---|---|
| | | Disk | clear Zone | |
| Compound | Cpd. No. | (μg/disk) | A. niger | A. flavus |
| CH₃SSCH₂OC(O)CH₃ | (2) | 100 | 2.3 | 2.3 |
| CH₃SSCH₂OC(O)CH₂CH₃ | (3) | 25 | 14.8 | 7.6 |
| CH₃SSCH₂OC(O)Ph | (4) | 25 | 6.1 | 8.4 |
| PhSSCH₂OC(O)CH₂CH₃ | (5) | 2.5 | 3.5 | 2.3 |

Compounds (4) and (5) in Table 6 are novel compounds.

TABLE 7

| | | | Diameter (mm) of | | |
|---|---|---|---|---|---|
| | Cpd | Dose | Clear Zone | | Appl'n |
| Compound | No. | (μg/disk) | A. niger | A. flavus | Solvent |
| (CH₃OC(O)CH₂S)₂ | (1) | 100 | 0 | 0 | Acetone |
| CH₃OC(O)CH₂SSCH₂OC(O)C₂H₅ | (2) | 0.25 | 3.0 | 2.3 | Acetone |
| (C₂H₅C(O)OCH₂S)₂ | (3) | 0.25 | 3.6 | 6.7 | Acetone |

TABLE 7

| | | | Diameter (mm) of | | |
|---|---|---|---|---|---|
| | Cpd | Dose | Clear Zone | | Appl'n |
| Compound | No. | (μg/disk) | A. niger | A. flavus | Solvent |
| C₂H₅OC(O)CH₂SSCH₂SO₂(C₆H₄)—p-CH₃ | (4) | 0.25 | 8.5 | 5.2 | Acetone |
| Amphotericin B | | 0.25 | 4.2 | 4.1 | DMSO |
| CH₃OC(O)CH₂SSCH₂OC(O)C₂H₅ | (2) | 0.25 | 11.7 | 7.9 | DMSO |
| (C₂H₅OC(O)CH₂S)₂ | (1) | 0.25 | 8.0 | 6.1 | DMSO |

Compounds (2), (3) and (4) of Table 7 are novel compounds. A novel method for preparing compounds (2) and (4) is described in detail in the section entitled “Preparatory Methods”. Compound (3) is prepared via a known method and this is detailed in the aforementioned section. Compound (1) is a known compound and the literature contains many references to standaard mathods that can be used to produce it.

Amphotericin B was the most potent of the 3 commercial antifungals tested (included Nystatin and Griseofulvin).

TABLE 8

| | | | Diameter (mm) of | | |
|---|---|---|---|---|---|
| | | Dose | Clear Zone | | |
| | Cpd | (μg/ | A. niger | | |
| Compound | No | disk) | A. flavus | | Reference |
| CH₃SO₂CH₂SSCH₃ | (2) | 25 | 2.7 | 3.8 | 2 |
| o-CH₃SS(C₆H₄)NO₂ | (3) | 10 | 2.8 | 1.9 | 3 |
| CH₃SSCH₃ | (10) | 100 | 0 | 0 | 3 |
| CH₃SO₂SCH₃ | (11) | 100 | 3.3 | 0 | — |
| CH₃CH₂SO₂SCH₃ | (5) | 50 | 2.0 | 1.3 | — |
| (C₆H₅)SSCH₃ | (12) | 100 | 0 | 0 | 3 |
| CH₃SO₂S(C₆H₅) | (4)* | 25 | 0 | 3.7 | — |
| p-CH₃(C₆H₄)SO₂SCH₃ | (13) | 25 | 1.4 | 3.6 | — |
| (C₆H₅)SS(C₆H₅) | (14) | 100 | 0 | 0 | 3 |
| (C₆H₅)SO₂S(C₆H₅) | (15) | 25 | 3.9 | 3.8 | — |
| p-CH₃(C₆H₄)SO₂S(C₆H₅) | (16) | 25 | 4.3 | 3.3 | — |
| o-CH₃SS(C₆H₄)CO₂CH₃ | (17) | 25 | 2.8 | 4.3 | 3 |
| o-CH₃SO₂S(C₆H₄)CO₂CH₃ | (18) | 100 | 0 | 0 | — |
| p-CH₃SS(C₆H₄)NO₂ | (7) | 10 | 3.8 | 4.0 | 3 |
| p-CH₃SO₂S(C₆H₄)NO₂ | (6) | 25 | 1.9 | 1.2 | — |
| p-CH₃(C₆H₄)SO₂S(C₆H₄)NO₂—p | (8) | 25 | 3.0 | 2.0 | — |
| CH₃SSCH₂CO₂CH₃ | (20) | 100 | 0 | 0 | 1 |
| p-CH₃(C₆H₄)SO₂SCH₂CO₂CH₃ | (9) | 50 | 2.9 | 1.7 | — |

*Compound (4) had no measureable fungitoxicity against A. niger at 100 μg/disk.

Thiosulfonates as Antifungal Agents

In exploring the development of new, potent antifungal disulphides (Baerlocher, F. J., Langler, R. F., Frederiksen, M. U., Georges, N. M., and Witherell, R. D., Aust. J. Chem., 1999, 52, 167, Langler, R. F., Ma [symbol] urrie, S. L., McNamara, R. A., and O'Conn [symbol] E., Aust. J. Chem., 52,1119 (1999), Baerlocner, F. J., Baerlocher, M. O., Langler, R. F., MacQuarrie, S. L., and Marchand, M. E., Aust. J. Chem., 531 (2000), and Baerlocher, F. J., Baerlocher, M. O., Chaulk, C. L., Langler, R. F., and O'Brien, E. M., Sulphur Letters—in press, the view that fungitoxicity is likely to be associated with biochemical sulfenylations accomplished by the present disulphides was adopted. Thus, enhancing toxicity has meant making disulphides which are progressively more electrophilic at disulphide sulfur. As an example, phenyl methyl disulphide exerts no observable fungitoxicity at 100 μg/disk (Baerlocher, F. J., Baerlocher, M. O., Langler, R. F., MacQuarrie, S. L., and Marchand, M. E., Aust. J. Chem., 53 1 (2000)) while o-nitrophenyl methyl disulphide (2) has pronounced toxicity at a dose of 10 μg/disk (see Table 8). Since thiosulfonates are well-known sulfenylating agents for mercaptide anions (see p. 323 of Bere, C. M., and Smiles, S., J. Chem. Soc., 1924, 125, 2361), selected thiosulfonates have been prepared and tested as potential fungitoxins.

Dimethyl disulphide (10) shows no antifungal behaviour, while the two closely related thiosulfonates (5) and (11) have measureable toxicity (vide Table 8). Methyl methanethiosulfonate (11) is the first compound examined which is effective against only one of the test fungi. Note that the enhanced toxicity of (5) is not unexpected since, up to a chain length of nine carbons, longer unbranced alkyl groups are known to enhance pharnacological effects (Silverman, R. B., ‘The Organic Chemistry of Drug Design and Drug Action’ p. 16 (Academic Press: San Diego 1992)).

For the series of compounds (4), (12), (13), the disulphide was inactive and the thiosulfonates antifungal. Interestingly, the thiosulfonate (4) killed only A. flavus in contrast to (11) which selectively inhibited the growth of A. niger.

Compounds (14)-(16) also demonstrated the superior antifungal potency of thiosulfonates relative to simple disulphides (see Table 1) but for this set, no selectivity for either of the fungi was observed.

Compound (17)(Table 8) was the first of the potent second-generation antifungal disulphides to be considered here. Perhaps surprisingly, the closely realted thiosulfonate (18) is not antifungal at all. Similar results (diminshed fungitoxicity for the thiosulfonates relative to the disulphide) were obtained for the set of compounds (6), (7), (8). It now appears that thiosulfonates tend to have moderate fungitoxicity—enhanced relative to inactive disulphides and diminished relative to more potent antifungal disulphides. The results in Table 8 open up the possibility that there might be a pharmacological equivalent to the well-known Reactivity Selectivity Principle in organic chemistry (Lowry, T. H., and Richarson, K. S., ‘Mechanism and Theory in Organic Chemistry’ 3rd. Ed., p. 148 (Harper and Row: New York 1987)). The pharmacological equivalent might assert that structural modifications which decrease the potency of a particular agent may be associated with enhanced selectivity in toxicity within a set of closely related organisms.

The interest in antifung [symbol] ulphides was encouraged by the recent [symbol] ervations (Pfaller, M., and Wenzell, R., Eur. J. Clin. Microbiol. Infect. Dis. 1992, 11, 287, Debono, M, and Gordee, R. S., Ann. Rev. Microbiol., 1994, 48, 471, and Sternberg, S., Science, 1994, 266, 1632) that fungal infections frequently prove to be lethal for immunocompromised patients. It has recently been learned that (19), a disulphide related to the potent aryl disulphide fungitoxins (e.g. (7) and (17) in Table 8), has been patented for use in inhibiting the production of Interleukin-1β and Tumor Necrosis Factor a (Katsuyama, K., Ariga, M., Saito, Y., Hatanaka, S., and Takahashi, T., U.S. Pat. No. 5,698,564 (1997)).

Finally, it has been reported earlier, that the α-ester disulphide (20) (see Table 8) had no antifungal activity (Baerlocher, F. J., Langler, R. F., Frederiksen, M. U., Georges, N. M., and Witherell, R. D., Aust. J. Chem., 1999, 52,167). Nonetheless, when tested, it showed promise as a lead compound in fighting leukemia (Wong, W. W. L., MacDonald, S., Langler, R. F., and Penn, L. Z., Anticancer Research 20:1367). The related thiosulfonate (9) shows clearly enhanced antifungal activity.

In terms of currently-available sulfur-rich antifungal compounds, thiosulfonates show intriguing selectivity.

Anti-cancer Activity

Summary of Results

The following tables group the tested compounds according to their demonstrated activities.

Compounds with No Antiproliferative Activity

| | |
|---|---|
| A | CH₃SO₂CH₃ |
| B | CH₃SO₂CH₂SCH₂SCH₃ |
| G | CH₃SO₂CH₂SCH₂SO₂CH₃ |
| J | CH₃SCH₂SCH₂SCH₃ |

Compounds with Antiproliferative Activity

| | |
|---|---|
| C | CH₃(C₆H₄)SO₂CH₂SSCH₃ |
| D | (C₆H₅)SS(C₆H₅) |
| E | CH₃SO₂CH₂SS(C₆H₅) |
| I | CH₃SSCH₂OC(O)CH₃ |
| K | CH₃SO₂CH₂SSCH₂CH₂CH₂CH₂CH₃ |
| L | CH₃SO₂S(C₆H₅) |
| M | p-NO₂(C₆H₄)SSCH₃ |
| N | CH₃SCH₂SSCH₃ |
| O | CH₃OC(O)CH₂SSCH₂C(O)OCH₃ |
| P | (C₆H₅)C(O)CH₂SSCH₃ |
| Q | (C₆H₅)SSCH₂OC(O)CH₂CH₃ |

Compounds P and Q are novel compounds. Compound Q is compound (5) in Table 6. A new method for preparing compound Q is described in detail in the section entitled “Preparatory Methods”.

Compounds with Antiproliferative, Tumour-specific Activity

| | |
|---|---|
| F | CH₃SO₂CH₂CH₂SSCH₃ |
| H | CH₃SSCH₂C(O)OCH₃ |

### MATERIALS AND METHODS

For references pertaining to methods, please refer to Wong, W. W-L., Macdonald, S., Langler, R. F., Penn, L. Z. Anticancer Res 20:1367, the disclosures of which are incorporated herein by reference. The number of times each experiment was performed is listed in individual compound data sets.

Cell Culture

All cell lines were assayed as asynchronously growing cells. Leukemic cell lines, OCI-AML-2, OCI-AML-3 (referred to hereafter as AML-2 and AML-3, respectively), NB-4, KK, B1, G2, and W1 were cultured in alpha-minimal essential medium (α-MEM) (Princess Margaret Hospital Media Services) supplemented with 10% fetal bovine serum (FBS) (Sigma, St. Louis, Mo.). Non-transformed, diploid fibroblast lines WI38 and IMR90, were cultured in α-MEM and MEM F-15, respectively supplemented with 10% FBS. Media for IMR90 cells was also supplemented with 1.5 g/L bicarbonate and 1 mM pyruvate. Breast tumour cell lines, MDA-231, SK-BR-3, MCF-7, ZR-751, and melanoma tumour cell lines, WM9, WM983, WM793, 1232, were grown in α-MEM supplemented with 10% fetal bovine serum. Prostate tumour cell lines, DU145 and PC-3, were grown in RPMI 1640 media supplemented with 10% FBS. All cell lines were cultured in the presence of penicillin/streptomycin. Mononucleated cells were isolated from normal bone marrow using Ficoll hypaque and then T-cell depleted. T-cell depletion was performed by incubating the mononucleated bone marrow cells in α-MEM at a concentration of 2×10⁷ cells/mL with 10% absor FBS and 10% (v/v) sheep red blood cells for 4 to 16 h. Absor FBS was prepared beforehand by heating FBS at 56° C. for 1 h, incubating with sheep red blood cells at 0.5% (v/v) for 1 h and then filtering the FBS with a 0.2 μM filter. The T-cell depleted, mononucleated cells were recovered using Ficoll hypaque and were maintained in α-MEM supplemented with 20% FBS and 10% 5637 [symbol] nditioned media. 5637 conditioned media [symbol] s previously harvested from confluent 5637 cells after a 3 day incubation. Normal bone marrow from bone marrow transplant donor was collected following informed consent according to institutional guidelines.

Organosulfur Compound Preparation for in vitro Testing

Approximately 20 mg of each compound was dissolved in 5 mL of ACS grade acetone (Sigma) using a 5 mL volumetric flask. Stock solutions of the compounds were stored in the dark at −20° C. Compounds were diluted prior to each experiment.

MTT Assay

Adherent cells were seeded at 67×10³ cells/mL in a 96 well plate (Falcon, Mississauga, Ontario) the day prior to exposure to compound. Suspension cells were seeded at 27×10⁴ cells/mL in a 96 well plate the day of exposure to compound. Mononucleated, T-cell depleted normal bone marrow cells were seeded at 67×10⁴ cells/mL in a 96 well plate the day of exposure to compound. Compounds and solvent control were added to cells and assayed in triplicate or sextuplet. Following 48 h of incubation at 37° C. with 5% CO₂, 40 μL of a 5 mg/mL solution of 3-[4,5-dimethylthiazol-2-yl]-2,5-diphenyltetrazolium bromide (MTT) substrate (Sigma) in Dulbecco's phosphate-buffered saline (D-PBS) was added. After 4 h of incubation at 37° C. with 5% CO₂, the resulting violet formazan precipitate was solubilized by the addition of 80 μL of a 0.01 M HCI, 10% sodium dodecyl sulfate (SDS; Sigma) solution overnight at 37° C. with 5% CO₂. The plates were then analyzed using the BioRad Benchmark Microplate Reader (BioRad Laboratories, Hercules, Calif.) at 570 nm to determine the optical density of the samples. MTT data was analyzed using Prism 3.0 (GraphPad Software, Inc., San Diego, Calif.) by the Chou-Talalay method. MTT graphs shown are a representative experiment.

Trypan Blue Exclusion Assay

Leukemic cells were seeded in a 24-well plate (Nunc, Naperville, Ill.) at 25×10⁴ cells/mL. Cells were then exposed in triplicate to solvent control or the approximate MTT50 concentration of compounds B, C, F, G or H at 10 μg/mL, or I at 5 μg/mL. The final solvent volume was 2.4 μL/mL of media for trypan blue exclusion assay. The compound was replenished after 48 h of treatment. Cell counts were evaluated using a 1:1 dilution of cell suspension in trypan blue (Gibco-BRL, Mississauga, Ontario, Canada). Viable and nonviable cells were counted using a hemocytometer. Cells which excluded trypan blue were counted as viable whereas stained cells were counted as nonviable. Trypan blue exclusion graphs shown are a representative experiment.

Fixed Propidium Lodide ( [symbol] ) Taining

Adherent cell lines were plated at 35×10⁴ cells/60 cm² dish the day before exposure to compound. Suspension cells were seeded at 25×10⁴ cells/mL in a 6 well dish (Falcon) the day of exposure to compound. Mononucleated, T-cell depleted normal bone marrow cells were seeded at 50×10⁴ cells/mL in a 6 well dish the day of exposure to compound. Cells were exposed to solvent control or approximate MTT50 and MTT30 concentrations of the compound. After 48 h of exposure to the compound, cells were harvested, fixed in 80% ethanol for 1 h on ice and labeled with 50 μg/mL propidium iodide (Sigma). Approximately 10⁶ cells were analyzed using a XL-MCL flow cytometer (Coulter Corporation, Miami, Fla.) and a FACScalibar cytometer (Becton Dickinson, San Jose, Calif.). Profiles shown are a representative.

Tdt-mediated dUTP-biotin Nick End-labeling (TUNEL)

Adherent cell lines were plated at 35×10⁴ cells/60 cm² dish the day before exposure to compound. Suspension cells were seeded at 25×10⁴ cells/mL in a 6 well dish (Falcon) the day of exposure to compound. Mononucleated, T-cell depleted normal bone marrow cells were seeded at 50×10⁴ cells/mL in a 6 well dish the day of exposure to compound. Cells were exposed to solvent control or approximate MTT50 and MTT30 concentrations of the compound. After 48 h of exposure to the compound, cells were harvested and fixed with a final concentration of 4% formaldehyde. Fixed cells were stored at −20° C. in 70% ethanol for no more than 5 days. Approximately 10⁵ cells were pelleted and labeled with 0.02 mM Biotin-dUTP and 12.5 U TdT enzyme in a 1× reaction buffer (200 mM potassium cacodylate, 25 mM Tris-HCl, 25 μg/mL bovine serum albumin, pH 6.6), 2.5 mM CoCl₂ and 0.01 mM dTTP (Roche Molecular Biochemicals, Laval, QC, Canada) for 45 min at 37° C. Samples were washed and incubated in 200 μL of 1:1000 fluorescein isothiocyanate (FITC)-conjugated avidin (Sigma) in 4×SSC, 5% skim milk powder and 0.05% Tween-20 (Sigma). Following 1 h of mixing at room temperature, the samples were washed and resuspended in 500 μL of D-PBS containing 2.5 μg/mL DNase-free RNase (Boehringer Mannheim) and 10 μg/mL PI. Following a 30 min incubation at room temperature, cells were analyzed using a XL-MCL flow cytometer (Coulter Corporation, Miami, Fla.) and a FACScalibar cytometer (Becton Dickinson, San Jose, Calif.). Profiles shown are representative of one experiment.

Materials and Methods

For references pertaining to methods, please refer to Wong, W. W-L., Macdonald, S., Langler, R. F., Penn, L. Z. Anticancer Res 20:1367, the disclosures of which are incorporated herein by reference. The number of times each experiment was performed is listed in individual compound data sets.

Cell culture

All cell lines were assayed as asynchronously growing cells. Leukemic cell lines, OCI-AML-2, OCI-AML-3 (referred to hereafter as AML-2 and AML-3, respectively), NB-4, KK, B1, G2, and W1 were cultured in alpha-minimal essential medium (α-MEM) (Princess Margaret Hospital Media Services) supplemented with 10% fetal bovine serum (FBS) (Sigma, St. Louis, Mo.). Non-transformed, diploid fibroblast lines WI38 and IMR90, were cultured in α-MEM and MEM F-1 5, respectively supplemented with 10% FBS. Media for IMR90 cells was also supplemented with 1.5 g/L bicarbonate and 1 mM pyruvate. Breast tumour cell lines, MDA-231, SK-BR-3, MCF-7, ZR-751, and melanoma tumour cell lines, WM9, WM983, WM793, 1232, were grown in α-MEM supplemented with 10% fetal bovine serum. Prostate tumour cell lines, DU145 and PC-3, were grown in RPMI 1640 media supplemented with 10% FBS. All cell lines were cultured in the presence of penicillin/streptomycin. Mononucleated cells were isolated from normal bone marrow using Ficoll hypaque and then T-cell depleted. T-cell depletion was performed by incubating the mononucleated bone marrow cells in α-MEM at a concentration of 2×10⁷ cells/mL and supplemented with 10% absor FBS (FBS heated at 56° C. for 1 h, incubated with sheep red blood cells at 0.5% (v/v) for 1 h and then filtered with a 0.2 μM filter) and 10% sheep red blood cells for 4 to 16 h. The T-cell depleted, mononucleated cells were recovered using Ficoll hypaque and were maintained in α-MEM supplemented with 20% FBS and 10% 5637 conditioned media. 5637 conditioned media was previously harvested from confluent 5637 cells after a 3 day incubation. Normal bone marrow from bone marrow transplant donor was collected following informed consent according to institutional guidelines.

Organosulfur Compound Preparation for in vitro Testing

Approximately 20 mg of each compound was dissolved in 5 mL of ACS grade acetone (Sigma) using a 5 mL volumetric flask. Stock solutions of the compounds were stored in the dark at −20° C. Compounds were diluted prior to each experiment.

MTT Assay

Adherent cells were seeded at 67×10³ cells/mL in a 96 well plate (Falcon, Mississauga, Ontario) the day prior to exposure to compound. Suspension cells were seeded at 27×10⁴ cells/mL in a 96 well plate the day of exposure to compound. Mononucleated, T-cell depleted normal bone marrow cells were seeded at 67×10⁴ cells/mL in a 96 well plate the day of exposure to compound. Compounds and solvent control were added to cells and assayed in triplicate or sextuplet. Following 48 h of incubation at 37° C. with 5% CO₂, 40 μL of a 5 mg/mL solution of 3-[4,5-dimethylthiazol-2-yl]-2,5-diphenyltetrazolium bromide (MTT) substrate (Sigma) in Dulbecco's phosphate-buffered saline (D-PBS) was added. After 4 h of incubation at 37° C. with 5% CO₂, the resulting violet formazan precipitate was solubilized by the addition of 80 μL of a 0.01 M HCl, 10% sodium dodecy [symbol] ate (SDS; Sigma) solution overnight at [symbol] with 5% CO₂. The plates were then analyzed using the BioRad Benchmark Microplate Reader (BioRad Laboratories, Hercules, Calif.) at 570 nm to determine the optical density of the samples. MTT data was analyzed using Prism 3.0 (GraphPad Software, Inc., San Diego, Calif.) by the Chou-Talalay method (Chou, T C and Talalay, P: Quantitative analysis of dose-effect relationships: the combined effects of multiple drugs or enzyme inhibitors. Adv Enzyme Reg 22: 27-55,1984, Chou, T C. (1991) in Synergism and antagonism in chemotherapy (T. C. Chou and D. C. Rideout, eds.), pp. 61-102, Academic Press, Inc., San Diego). MTT graphs shown are a representative experiment.

Trypan Blue Exclusion Assay

Leukemic cells were seeded in a 24-well plate (Nunc, Naperville, Ill.) at 25×10⁴ cells/mL. Cells were then exposed in triplicate to solvent control or the approximate MTT50 concentration of compounds F (10 μg/mL), G (10 μg/mL) or 1 (5 μg/mL). The final solvent volume was 2.4 μL/mL of media for trypan blue exclusion assay. The compound was replenished after 48 h of treatment. Cell counts were evaluated using a 1:1 dilution of cell suspension in trypan blue (Gibco-BRL, Mississauga, Ontario, Canada). Viable and nonviable cells were counted using a hemocytometer. Cells which excluded trypan blue were counted as viable whereas stained cells were counted as nonviable. Trypan blue exclusion graphs shown are a representative experiment.

Fixed Propidium Iodide (PI) Staining

Adherent cell lines were plated at 35×10⁴ cells/60 cm2 dish the day before exposure to compound. Suspension cells were seeded at 25×10⁴ cells/mL in a 6 well dish (Falcon) the day of exposure to compound. Mononucleated, T-cell depleted normal bone marrow cells were seeded at 50×10⁴ cells/mL in a 6 well dish the day of exposure to compound. Cells were exposed to solvent control or approximate MTT50 and MTT30 concentrations. After 48 h of exposure to the compound, cells were harvested, fixed in 80% ethanol for 1 h on ice and labeled with 50 μg/mL propidium iodide (Sigma). Approximately 10⁶ cells were analyzed using a XL-MCL flow cytometer (Coulter Corporation, Miami, Fla.) and a FACScalibar cytometer (Becton Dickinson, San Jose, Calif.). Profiles shown are a representative. Cell viability counts and Pi staining were performed as previously described (Dimitroulakos, J, Nohynek, D, Backway, K L, Hedley, D W, Yeger, H, Freedman, M H, Minden, M D and Penn, L Z: Increased sensitivity of acute myeloid leukemias to lovastatin-induced apoptosis: A potential therapeutic approach. Blood 93: 1308-18, 1999).

Tdt-mediated dUTP-biotin Nick End-labeling (TUNEL)

Adherent cell lines were plated at 35×10⁴ cells/60 cm² dish the day before exposure to compound. Suspension cells were seeded at 25×10⁴ cells/mL in a 6 well dish (Falcon) the day of exposure to compound. Mononucleated, T-cell depleted normal bone marrow cells were seeded at 50×10⁴ cells/mL in a 6 well dish the day of exposure to compound. [symbol] s were exposed to solvent control or appr [symbol] nate MTT50 and MTT30 concentrations of compound. After 48 h of exposure to the compound, cells were harvested and fixed with a final concentration of 4% formaldehyde. Fixed cells were stored at −20° C. in 70% ethanol for no more than 5 days. Approximately 10⁵ cells were pelleted and labeled with 0.02 mM Biotin-dUTP and 12.5 U TdT enzyme in a 1× reaction buffer (200 mM potassium cacodylate, 25 mM Tris-HCl, 25 μg/mL bovine serum albumin, pH 6.6), 2.5 mM CoCl₂ and 0.01 mM dTTP (Roche Molecular Biochemicals, Laval, QC, Canada) for 45 min at 37° C. Samples were washed and incubated in 200 μL of 1:1000 fluorescein isothiocyanate (FITC)-conjugated avidin (Sigma) in 4×SSC, 5% skim milk powder and 0.05% Tween-20 (Sigma). Following 1 h of mixing at room temperature, the samples were washed and resuspended in 500 μL of D-PBS containing 2.5 μg/mL DNase-free RNase (Boehringer Mannheim) and 10 μg/mL Pl. Following a 30 min incubation at room temperature, cells were analyzed using a XL-MCL flow cytometer (Coulter Corporation, Miami, Fla.) and a FACScalibar cytometer (Becton Dickinson, San Jose, Calif.). Profiles shown are representative of one experiment.

### BRIEF DESCRIPTION OF THE DRAWINGS

The accompanying drawings are used to illustrate the invention only and should not be used to limit the scope of the claims. The letter prefix to each of the Table or Figure numbers corresponds to a respective compound. The Tables and Figures have corresponding labels. Thus, Compound A data can be found in Table A-1 which corresponds to Figure A-1. In the accompanying Figures,

FIG. A-1 illustrates a MTT Assay with Leukemic cell lines, WI38 and normal bone marrow;

FIG. A-2 illustrates Fixed Propidium Iodide Profiles;

FIG. B-1 illustrates a MTT Assay with Leukemic cell lines, WI38 and normal bone marrow;

FIG. B-2 illustrates Trypan Blue Exclusion Assay;

FIG. B-3 illustrates Fixed Propidium Iodide Profiles;

FIG. B-4 illustrates TUNEL Profiles;

FIG. C-1 illustrates a MTT Assay with Leukemic cell lines, WI38 and normal bone marrow;

FIG. C-2 illustrates a Trypan Blue Exclusion Assay;

FIG. C-3 illustrates Fixed Propidium Iodide Profiles;

FIG. C-4 illustrates TUNEL Profiles;

FIG. D-1 illustrates a MTT Assay with Leukemic cell lines, WI38 and normal bone marrow;

FIG. D-2 illustrates Fixed Propidium Iodide Profiles;

FIG. E-1 illustrates MTT Assay with Leukemic cell lines, WI38 [symbol] and normal bone marrow;

FIG. E-2 illustrates Fixed Propidium Iodide Profiles;

FIG. F-1 illustrates a MTT Assay with Leukemic cell lines, WI38 and normal bone marrow;

FIG. F-2 illustrates a Trypan Blue Exclusion Assay;

FIG. F-3 illustrates Fixed Propidium Iodide Profiles;

FIG. F-4 illustrates TUNEL Profiles;

FIG. G-1 illustrates a MTT Assay with Leukemic cell lines, WI38 and normal bone marrow;

FIG. G-2 illustrates a Trypan Blue Exclusion Assay;

FIG. G-3 illustrates Fixed Propidium Iodide Profiles;

FIG. G-4 illustrates TUNEL Profiles;

FIG. H-1 illustrates a MTT Assay with Leukemic cell lines, WI38 and normal bone marrow;

FIG. H-2 illustrates a Trypan Blue Exclusion Assay;

FIG. H-3 illustrates Fixed Propidium Iodide Profiles;

FIG. H-4 illustrates TUNEL Profiles;

FIG. I-1 illustrates a MTT Assay with Leukemic cell lines, WI38 and normal bone marrow;

FIG. I-2 illustrates a Trypan Blue Exclusion Assay;

FIG. I-3 illustrates Fixed Propidium Iodide Profiles;

FIG. I-4 illustrates TUNEL Profiles;

FIG. J-1 illustrates a MTT Assay with Leukemic cell lines, WI38 and normal bone marrow;

FIG. J-2 illustrates Fixed Propidium Iodide Profiles;

FIG. K-1 illustrates a MTT Assay;

FIG. K-2 illustrates a MTT Assay with Normal Bone Marrow;

FIG. L-1 illustrates a MTT Assay;

FIG. L-2 illustrates a MTT Assay with Normal Bone Marrow;

FIG. M-1 illustrates a MTT Assay;

FIG. M-2 illustrates a MTT Assay with Normal Bone Marrow;

FIG. M-3 illustrates Fixed Propidium Iodide Profiles;

FIG. N-1 illustrates a MTT Assay;

FIG. N-2 illustrates a MTT Assay with Normal Bone Marrow;

FIG. N-3 illustrates Fixed Propidium Iodide Profiles;

FIG. N-4 illustrates TUNEL Profiles;

FIG. O-1 illustrates a MTT Assay;

FIG. O-2 illustrates a MTT Assay with Normal Bone Marrow;

FIG. P-1 illustrates a MTT Assay;

FIG. P-2 illustrates a MTT Assay with Normal Bone Marrow,

FIG. P-3 illustrates Fixed Propidium Iodide Profiles;

FIG. P-4 illustrates TUNEL Profiles;

FIG. Q-1 illustrates a MTT Assay;

FIG. Q-2 illustrates a MTT Assay with Normal Bone Marrow;

FIG. Q-3 illustrates a MTT Assay with Breast/Prostate/Melanoma cell lines;

FIG. Q-4 illustrates Fixed Propidium Iodide Profiles;

FIG. 0-5 illustrates TUNEL Profiles.

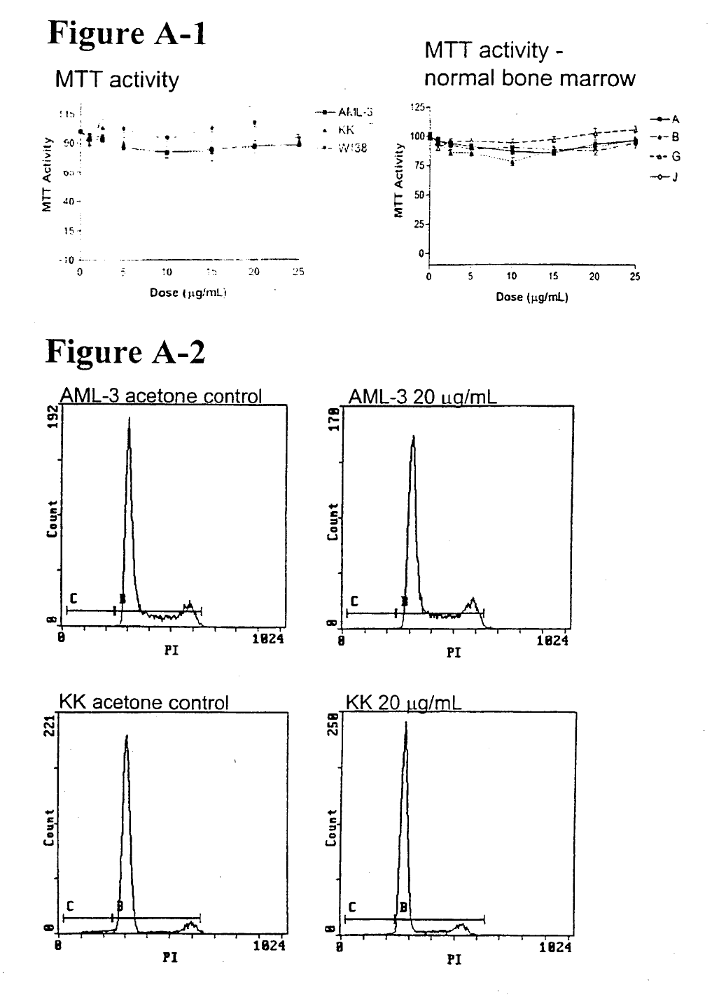

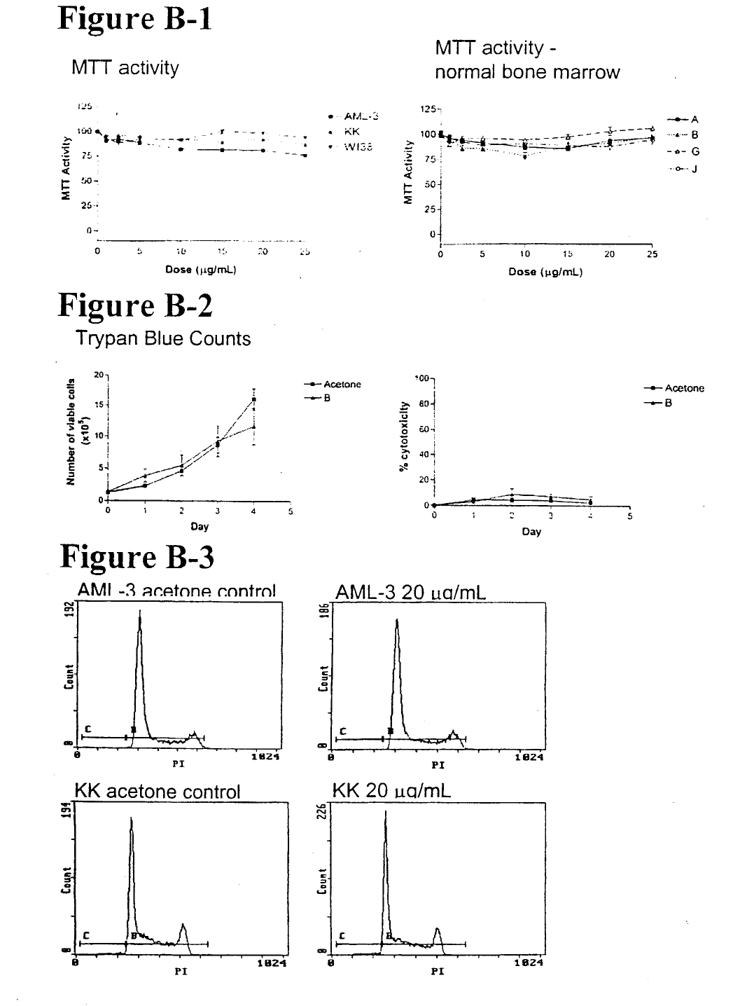

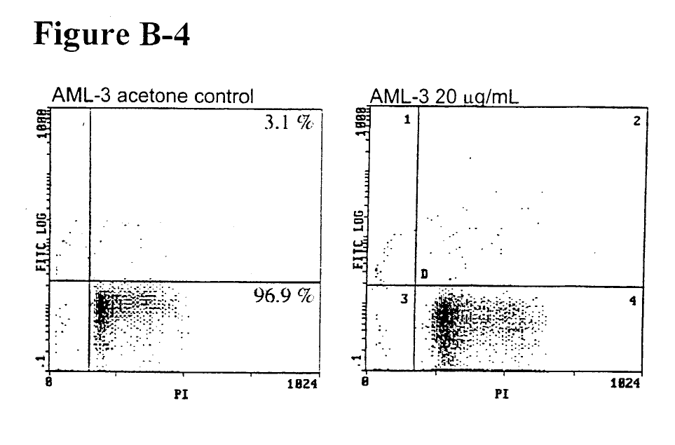

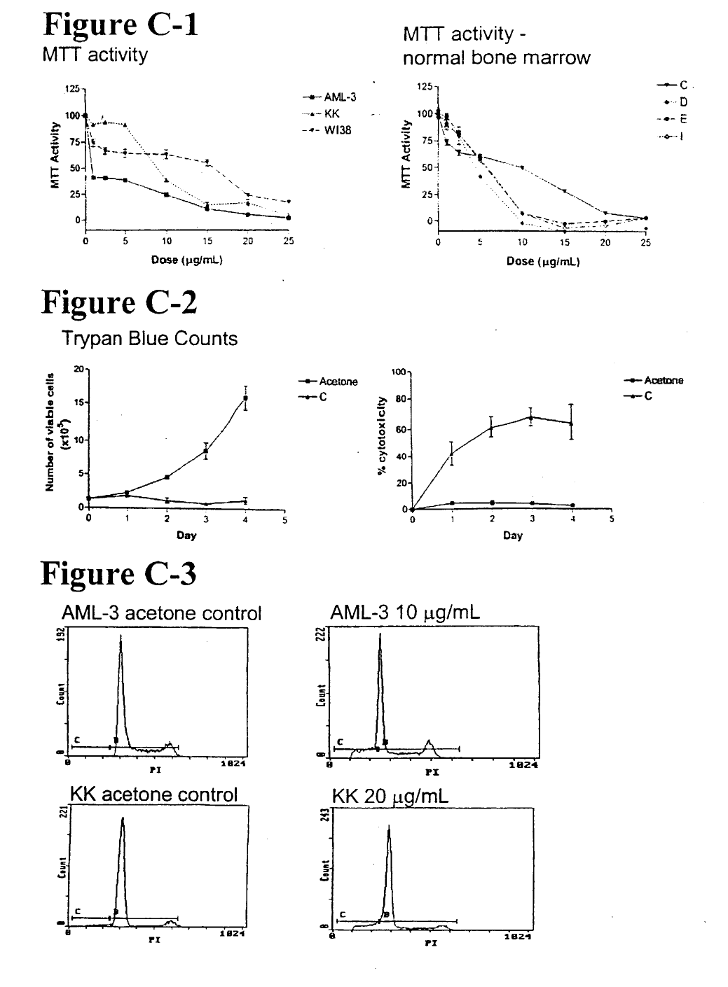

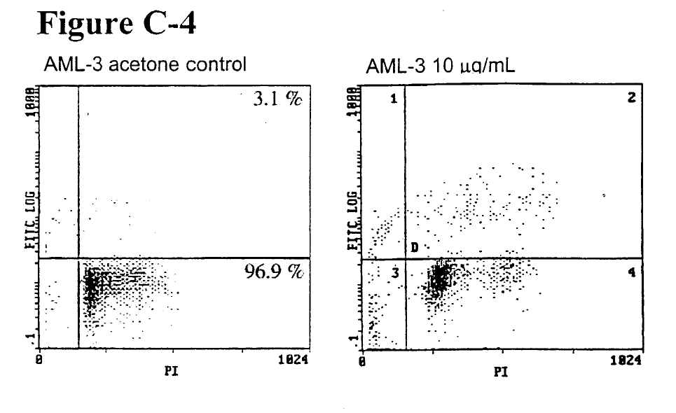

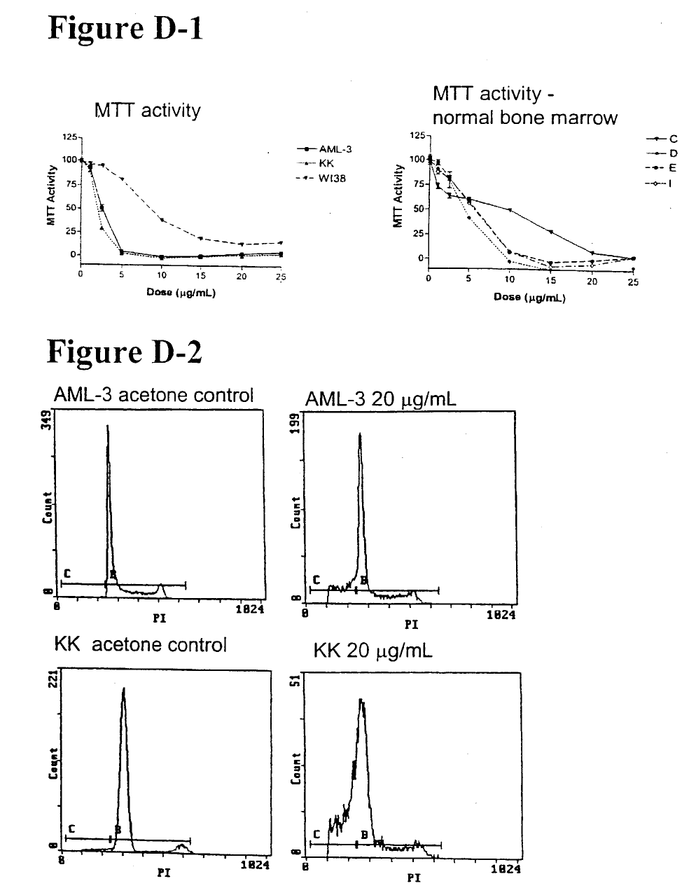

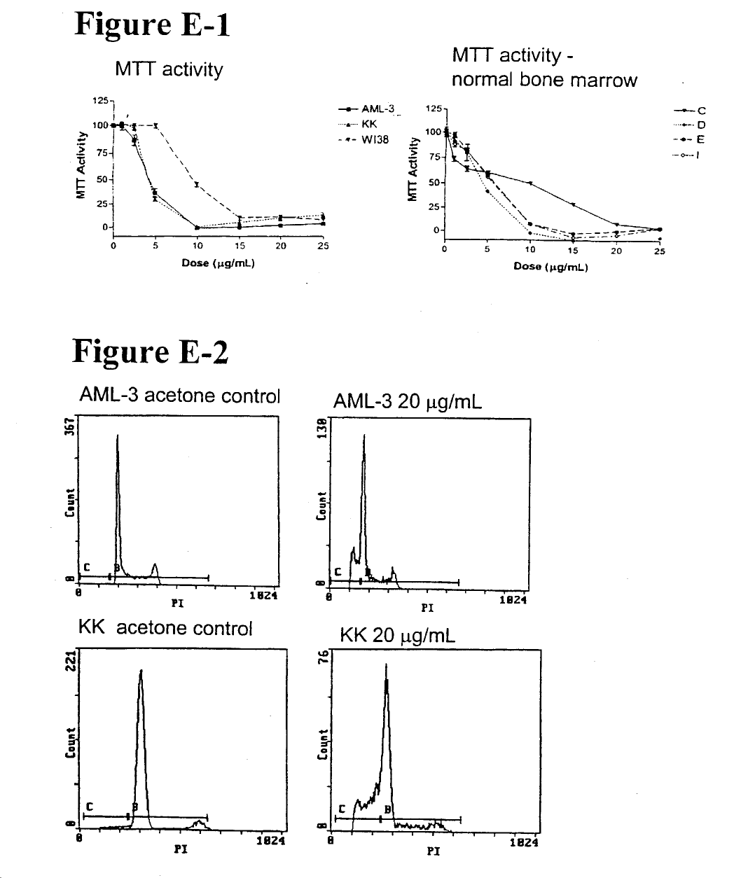

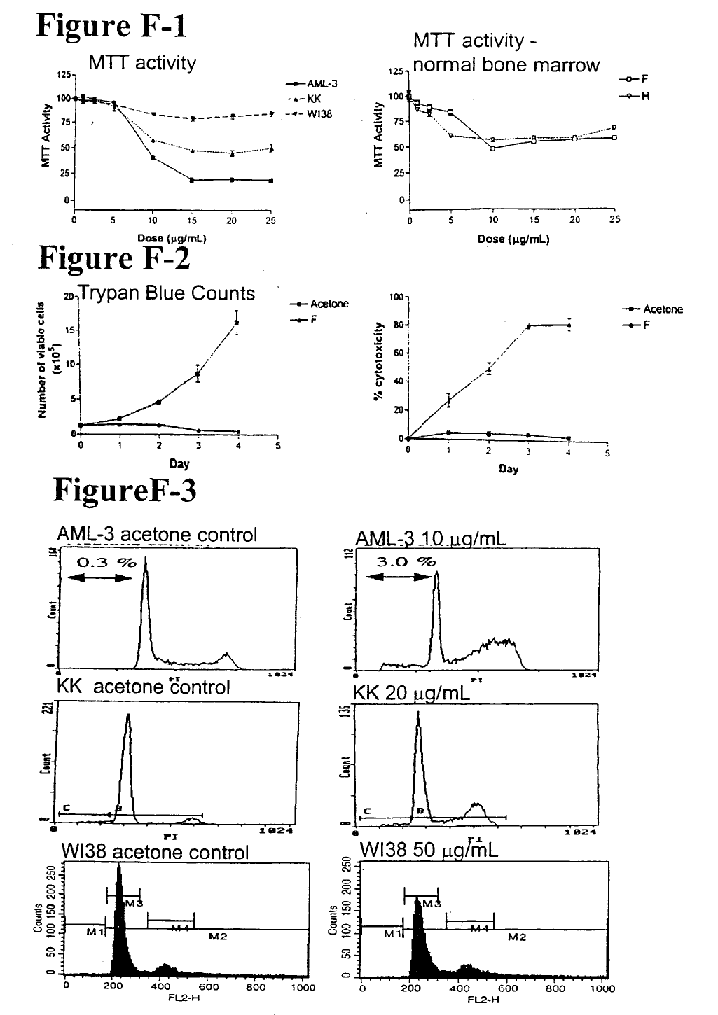

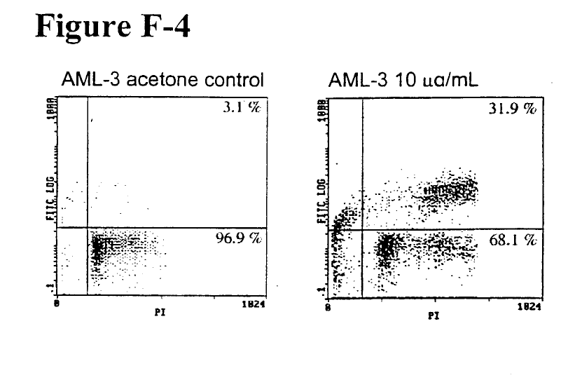

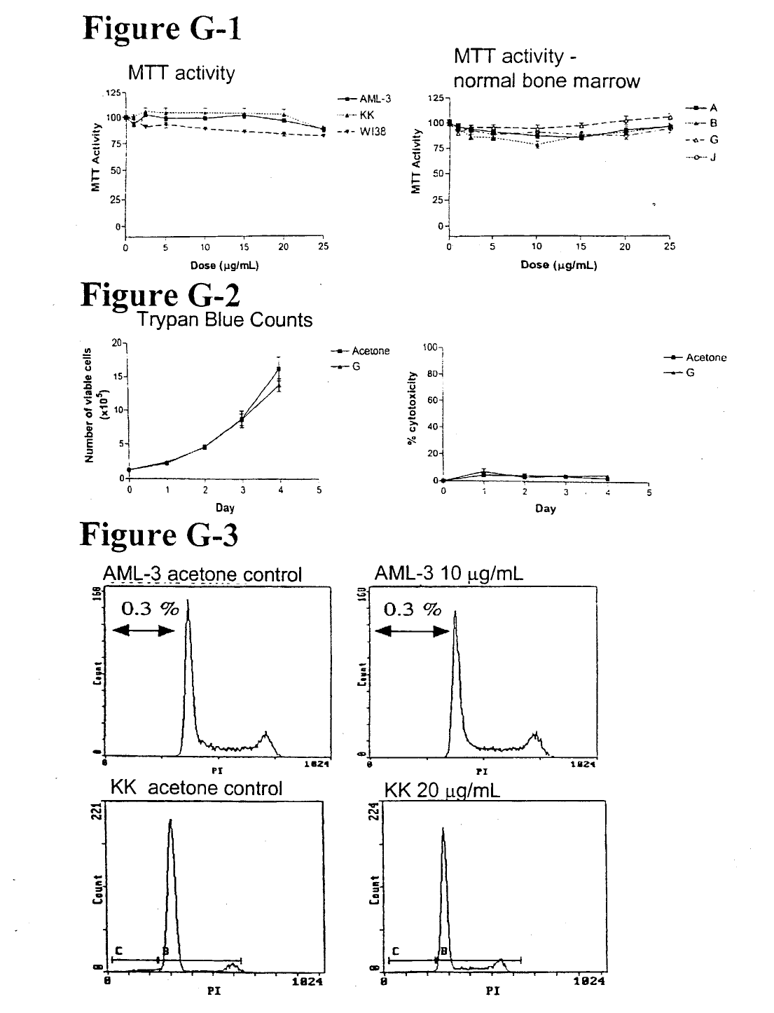

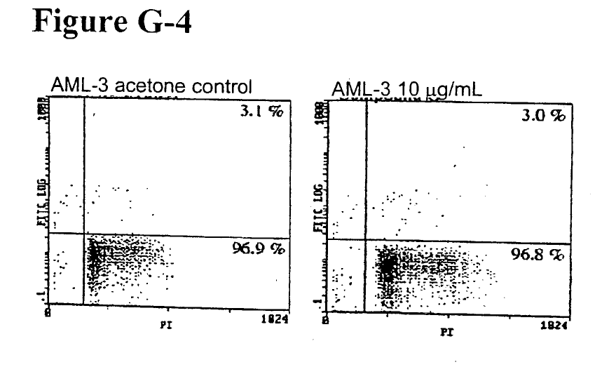

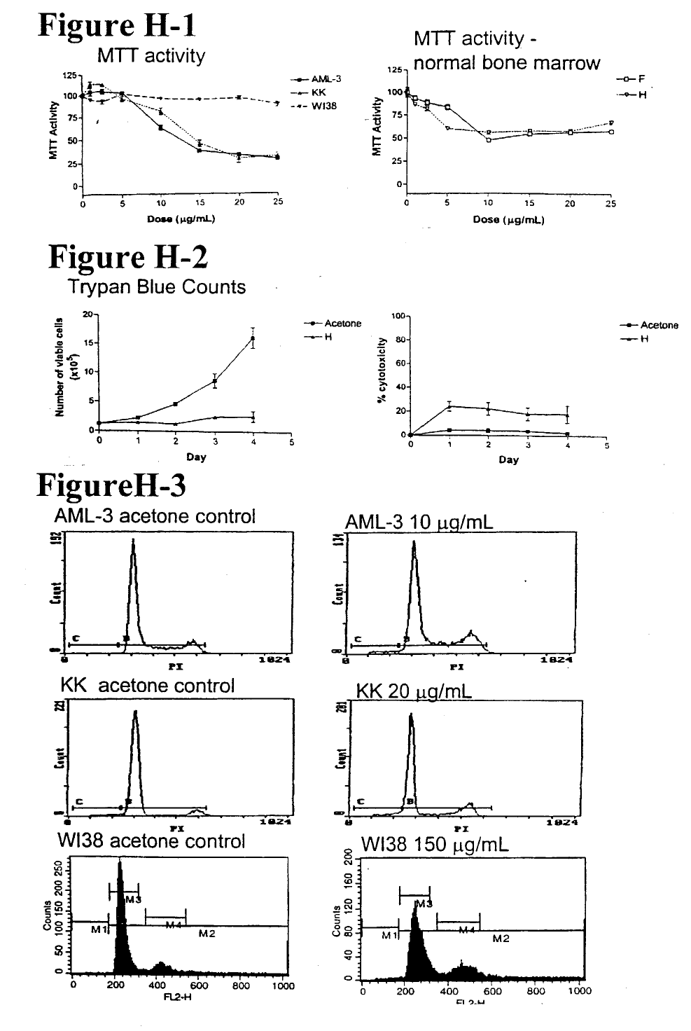

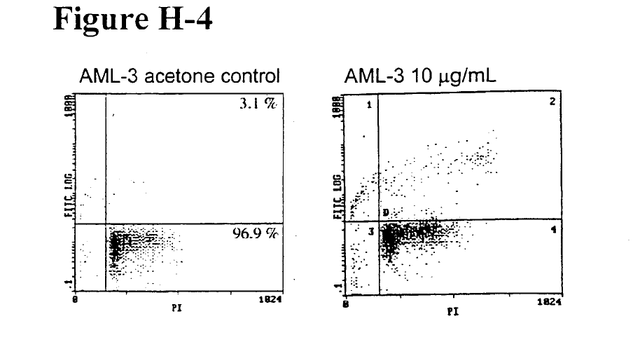

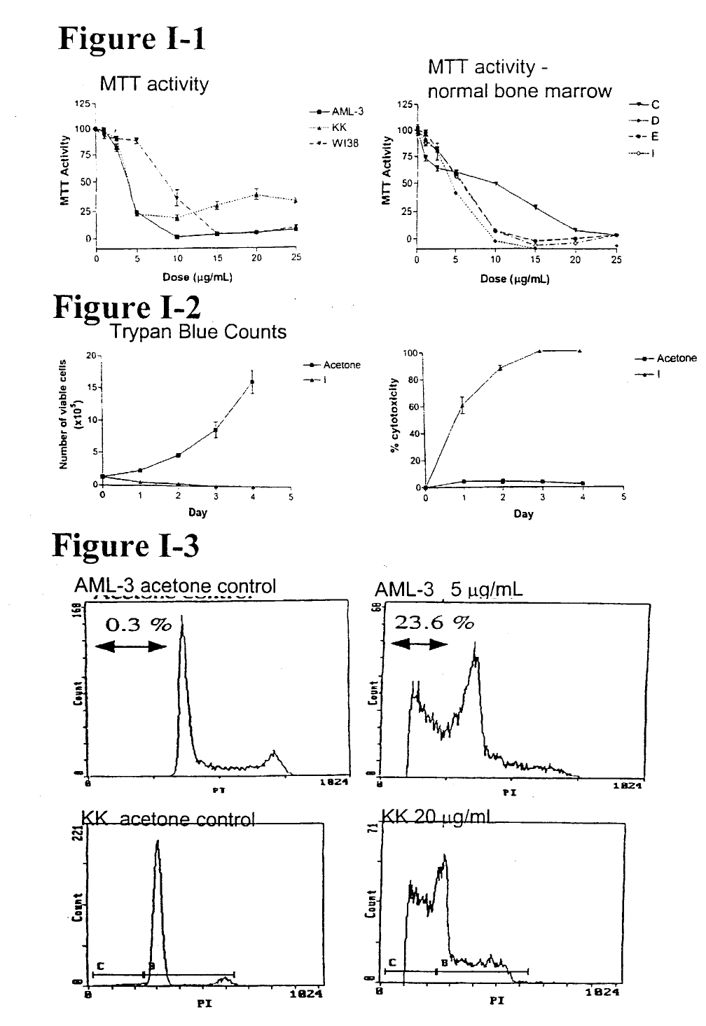

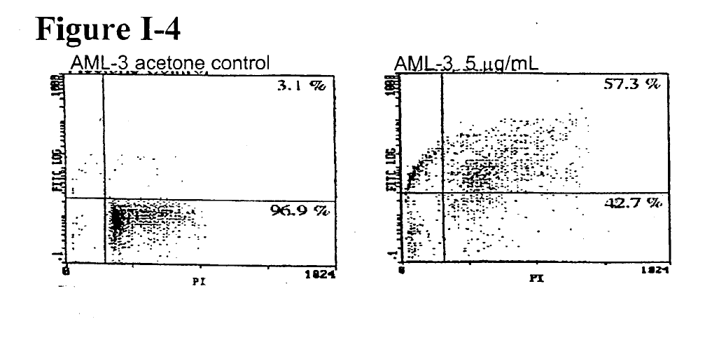

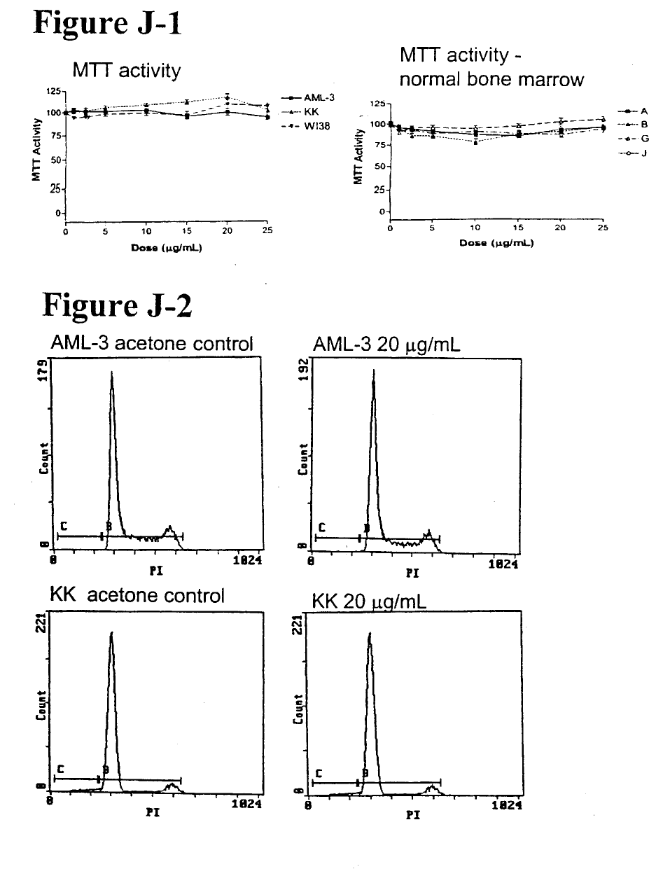

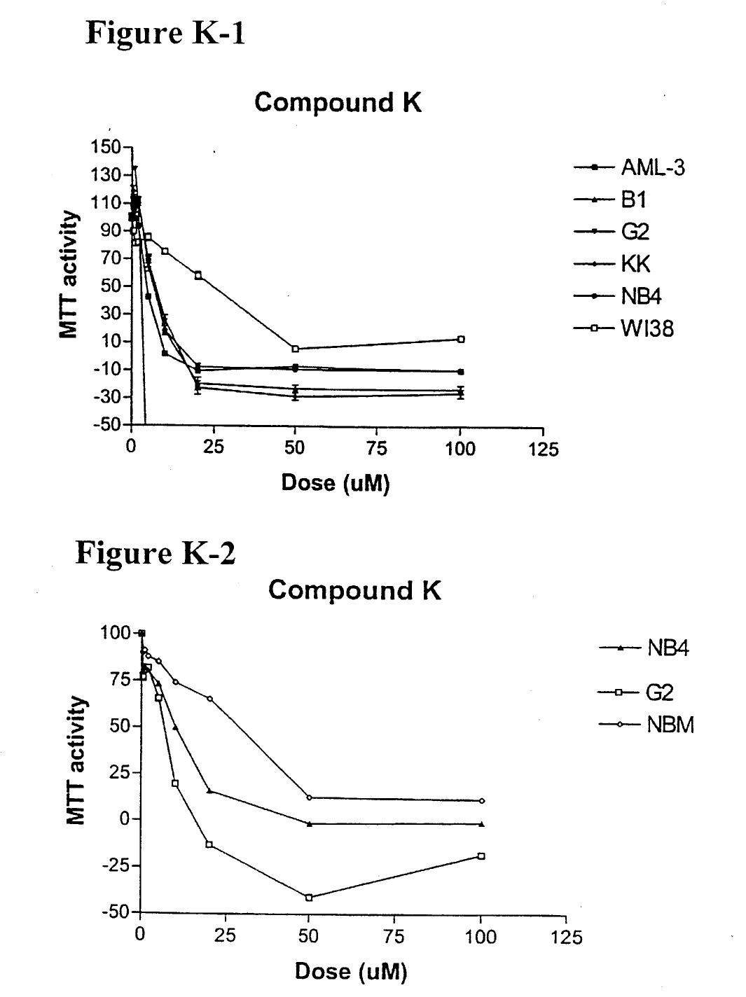

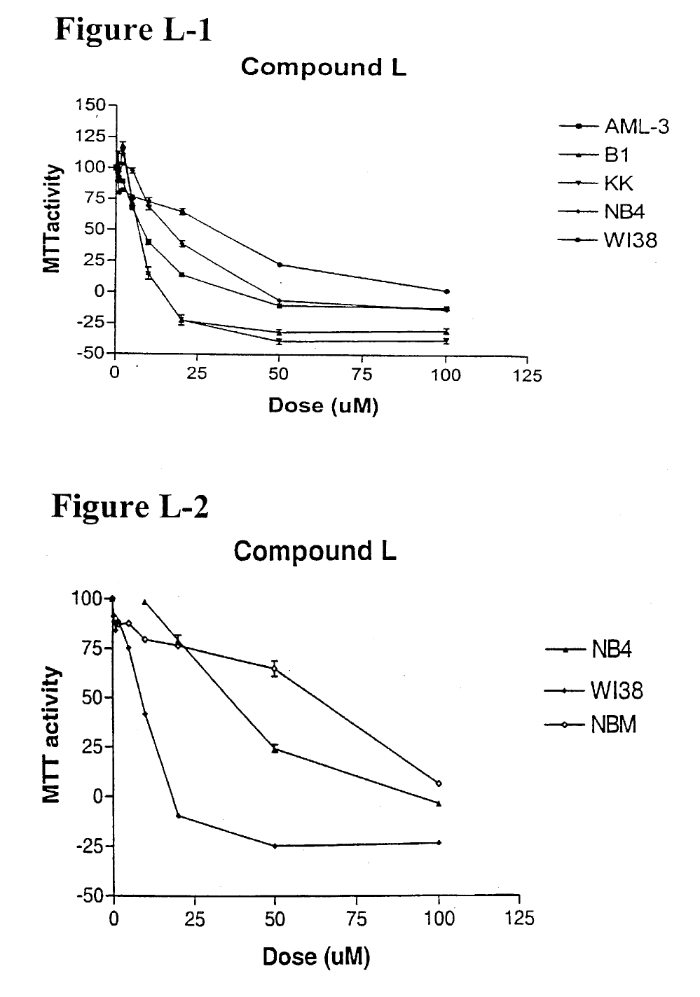

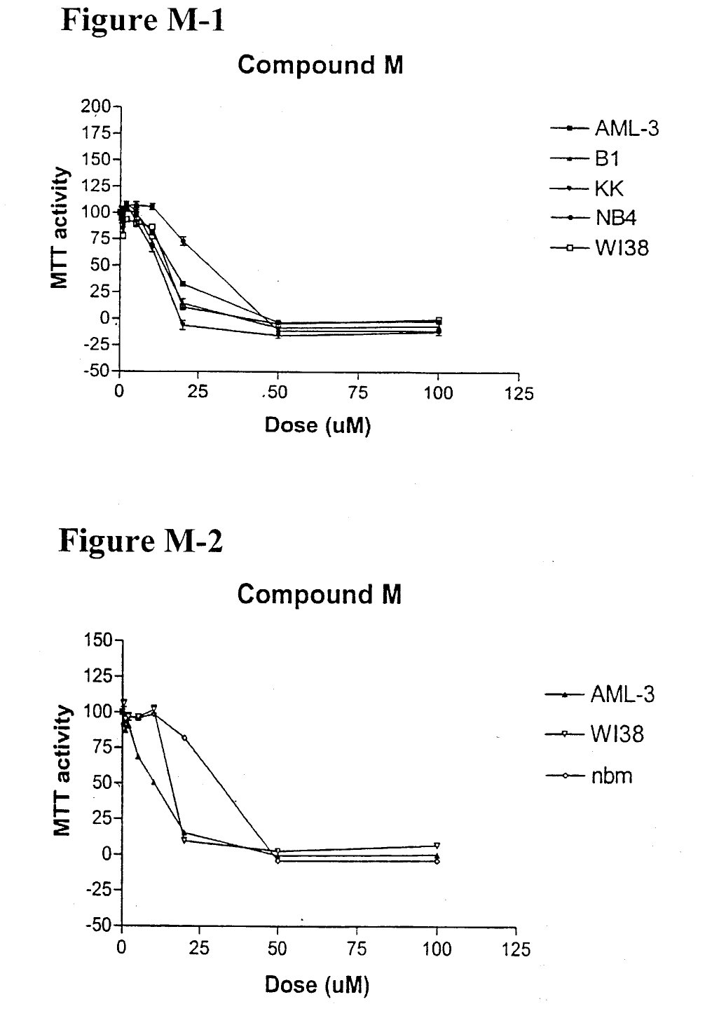

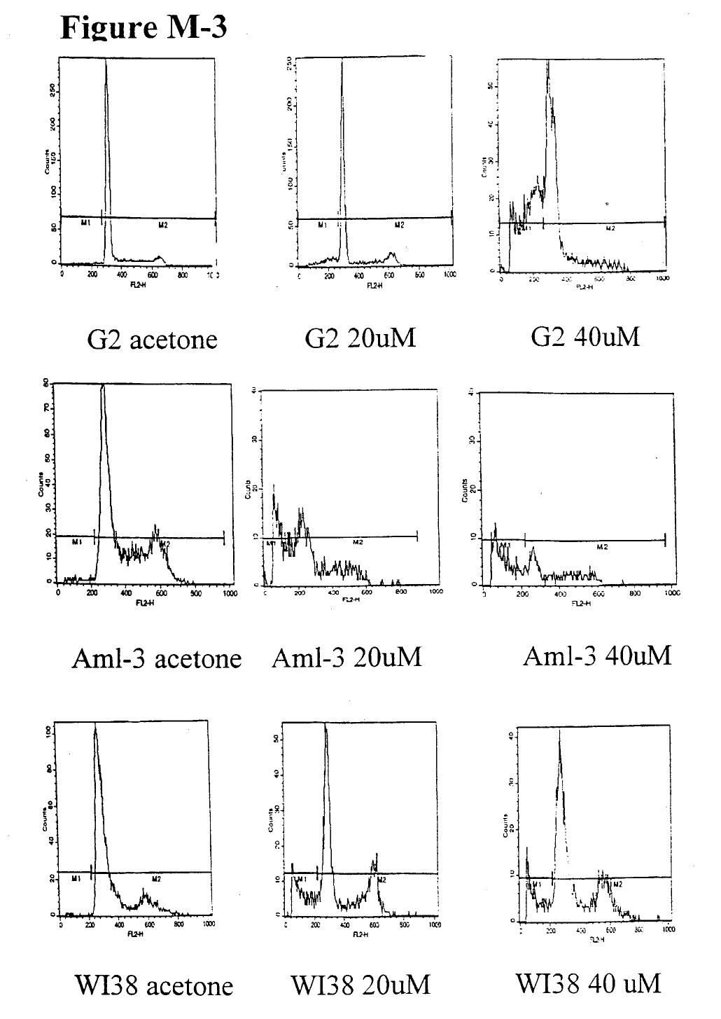

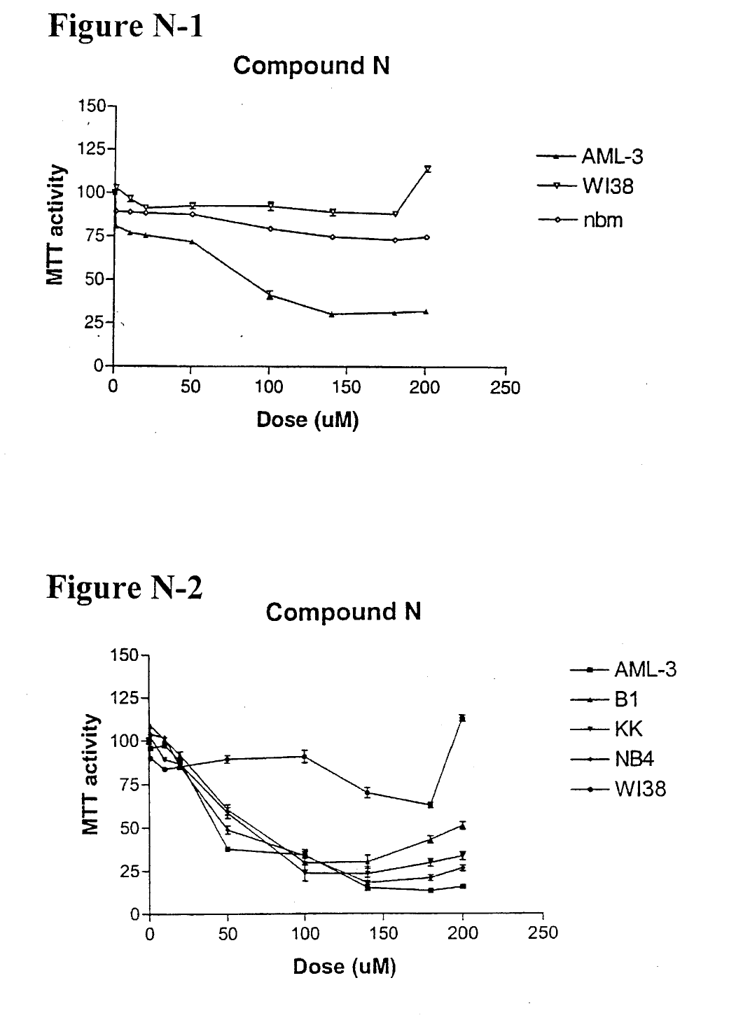

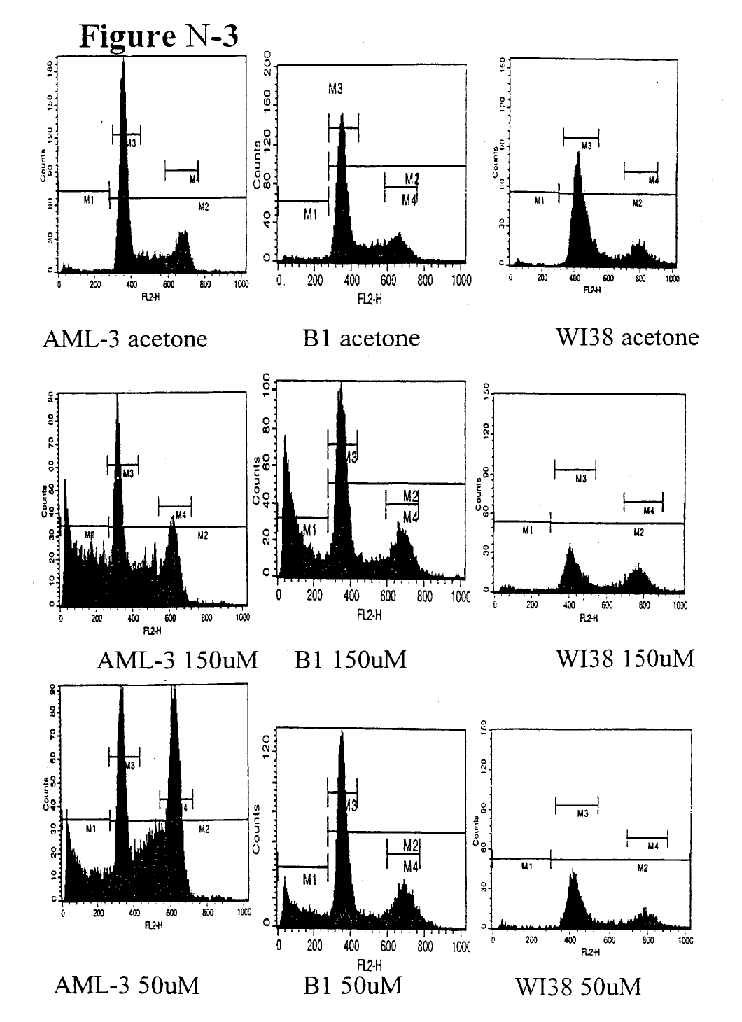

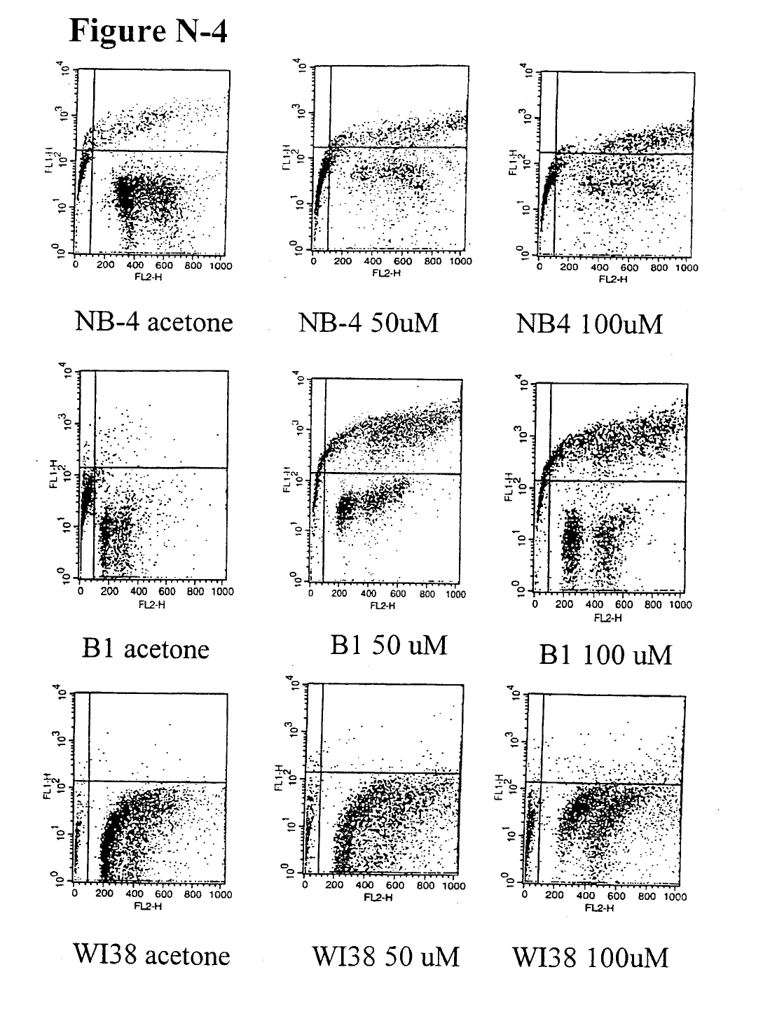

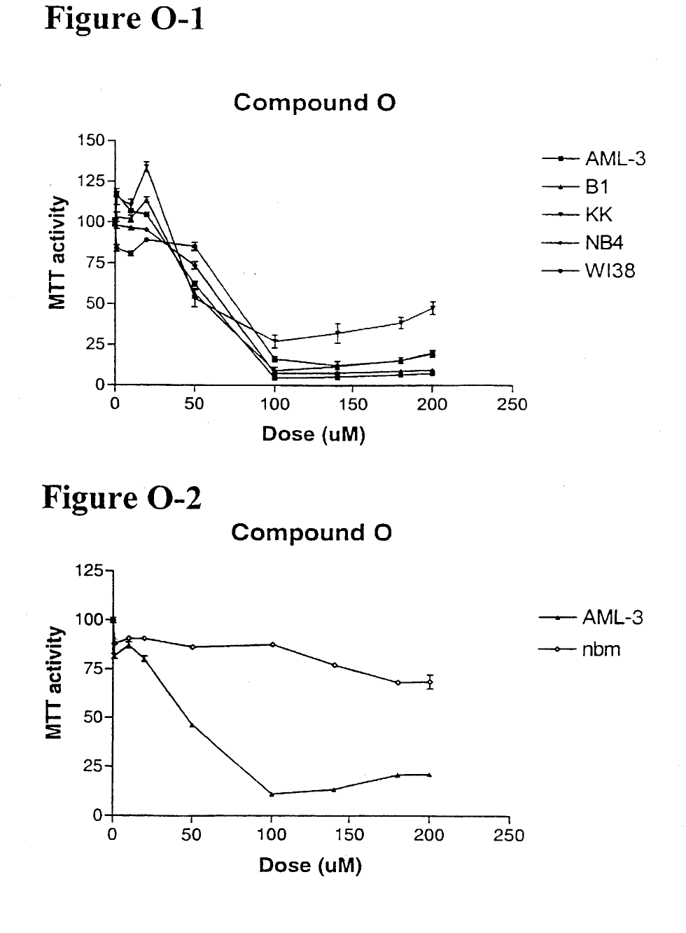

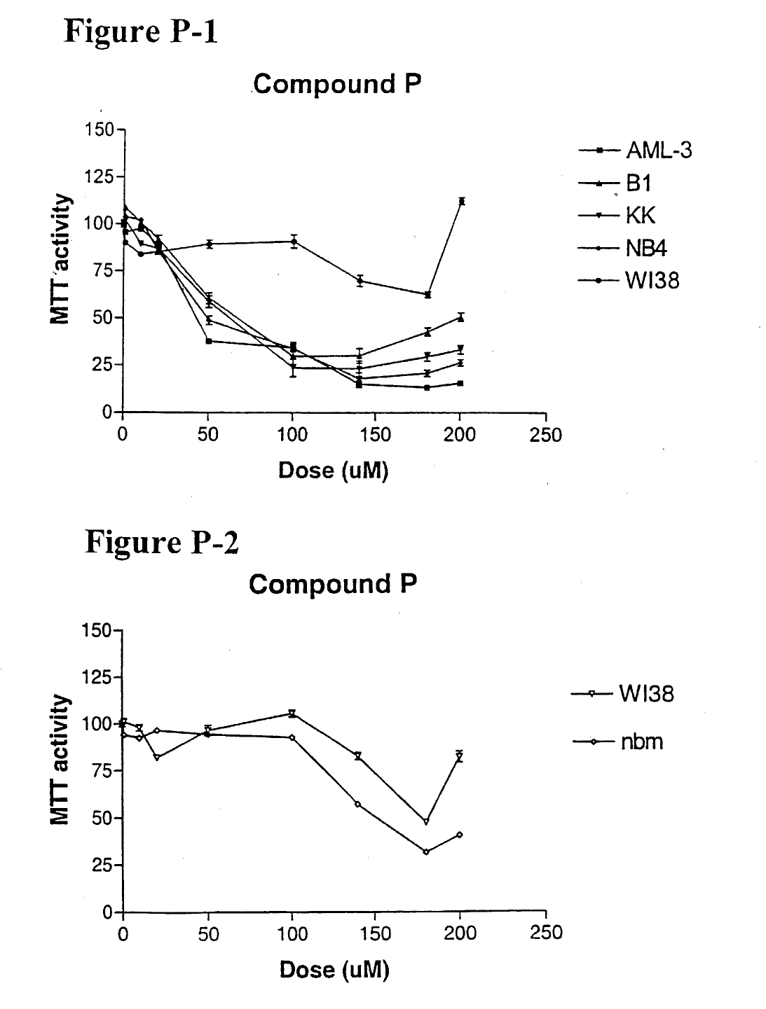

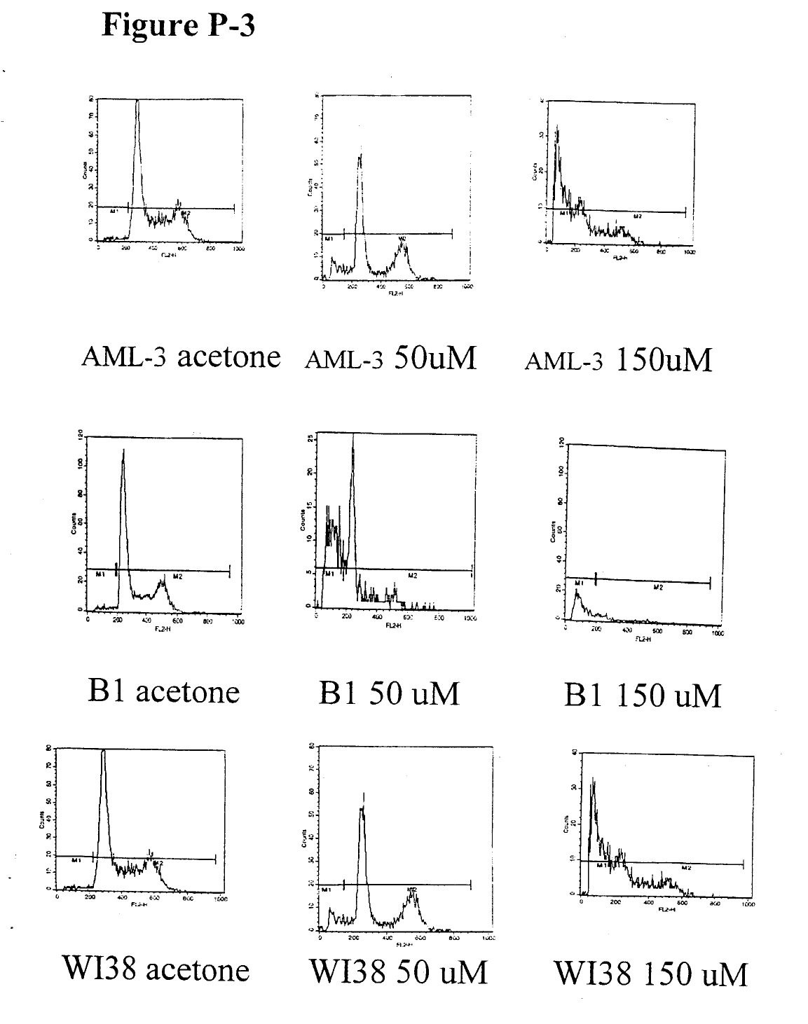

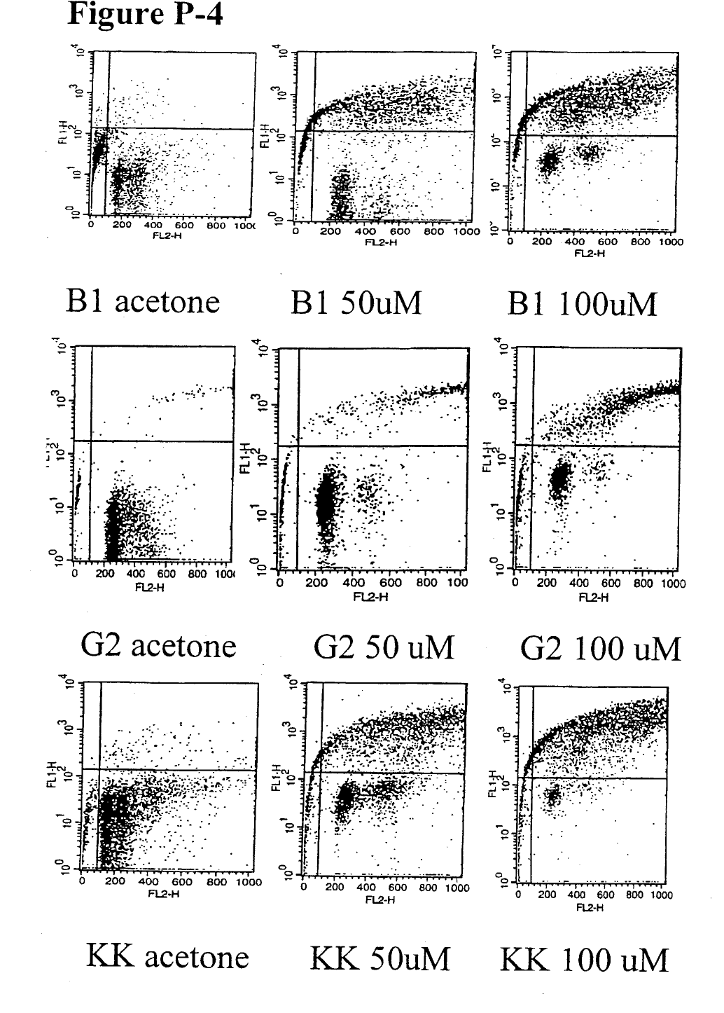

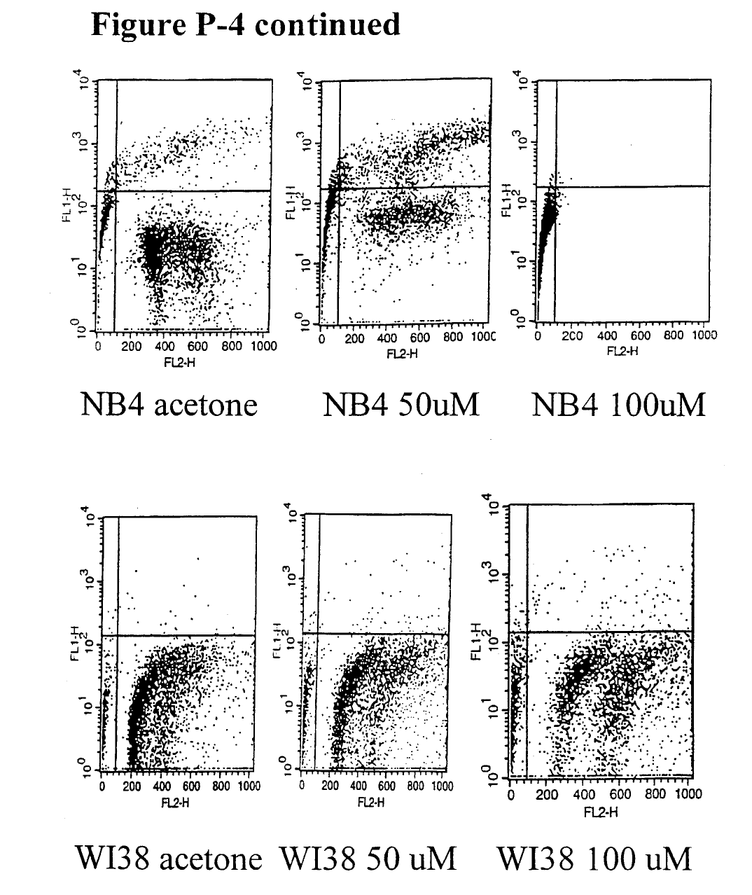

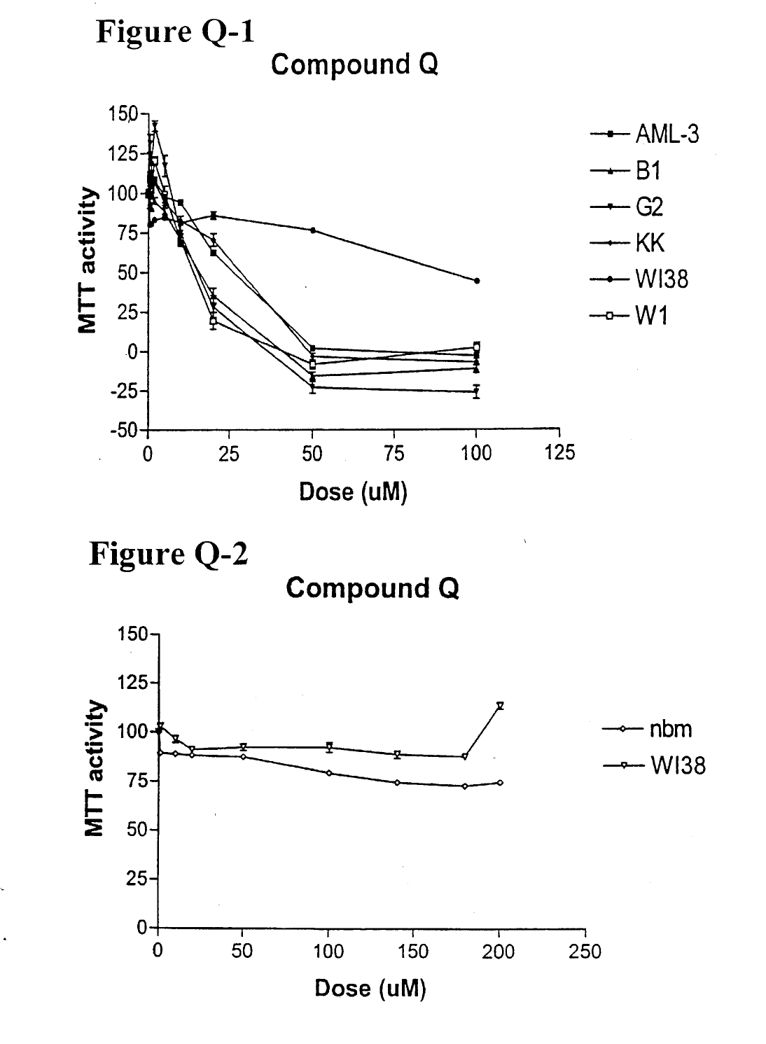

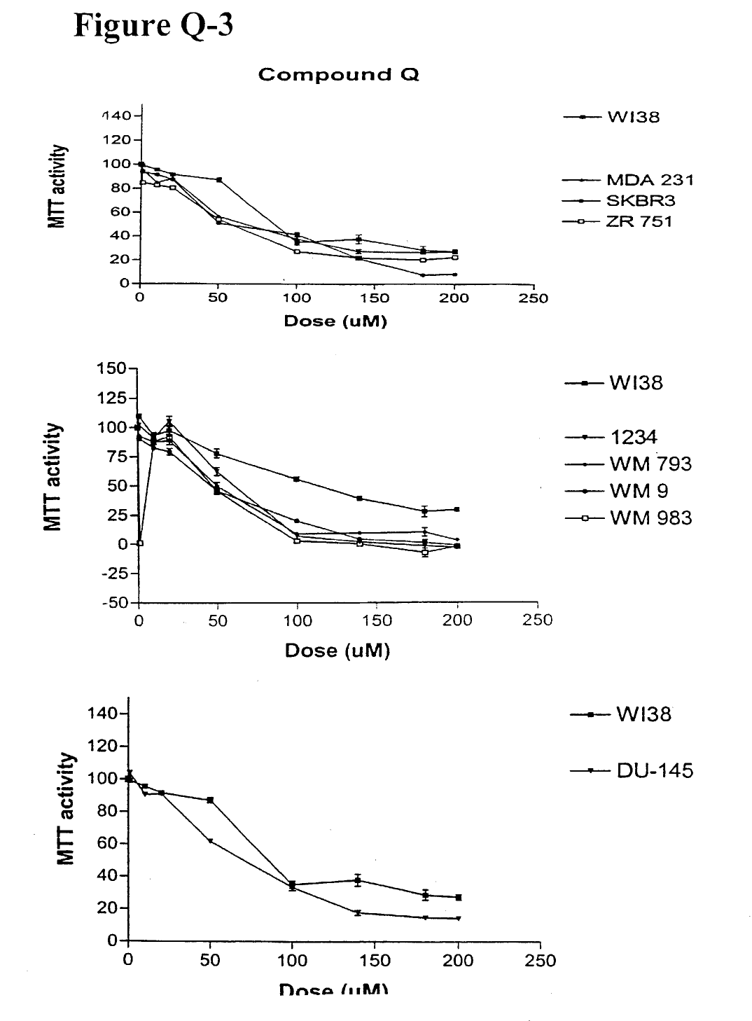

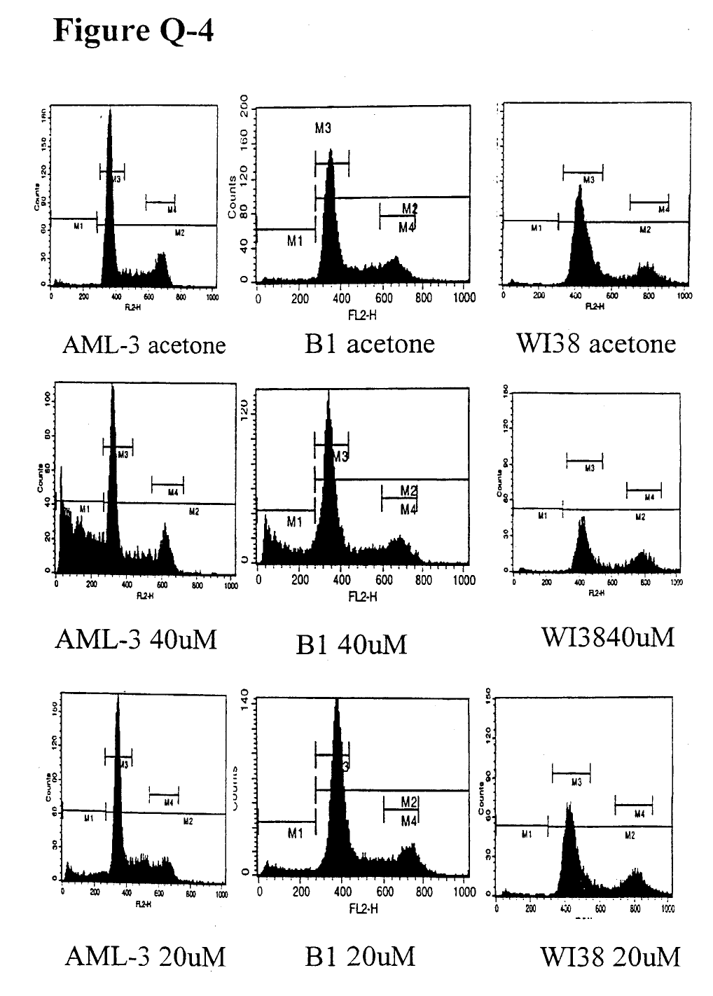

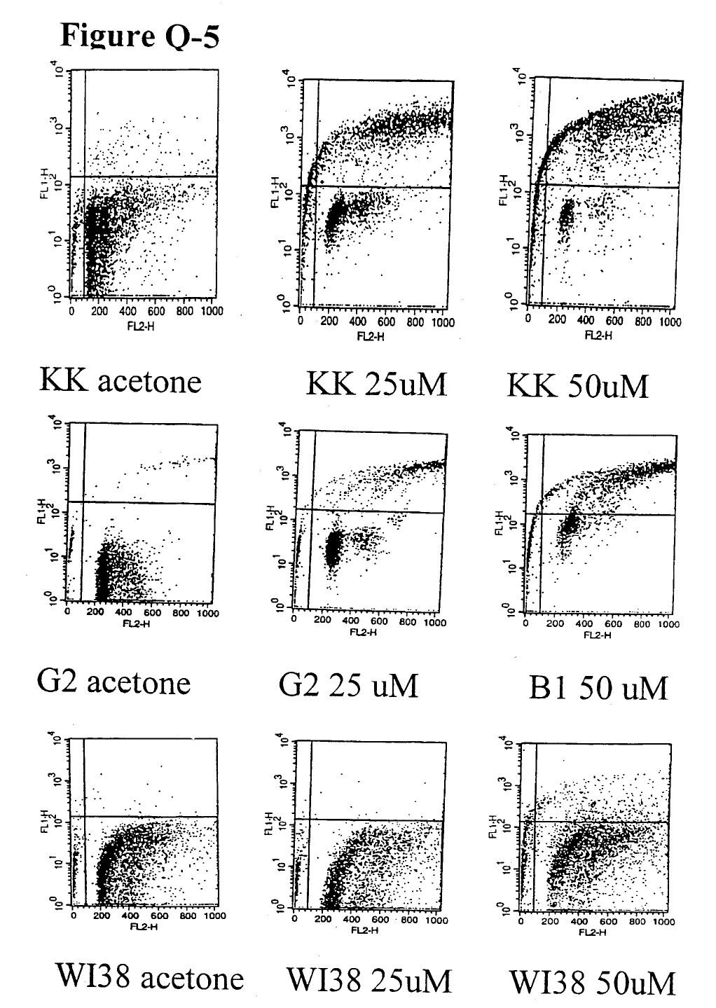

TABLE A-1

| | | |
|---|---|---|
| | MTT50 | Number of |
| Cell line | uM | Experiments |

Leukemic

| | | |
|---|---|---|
| AML-3 | >265 (25 ug/mL) | 6 |
| KK | >265 (25 ug/mL) | 6 |

Normal

| | | |
|---|---|---|
| WI38 | >265 (25 ug/mL) | 6 |
| Normal bone marrow | >265 (25 ug/mL) | 3 |

TABLE A-2

| | Flow Cytometry | | | |
|---|---|---|---|---|
| | Fixed PI | | | |
| | | | Acetone ctrl | Number of |
| Cell line | Dose (uM) | % pre-G1 | % pre-G1 | Experiments |

Leukemic

| | | | | |
|---|---|---|---|---|
| AML-3 | 212 (20 ug/mL) | 0.1 | 0.8 | 3 |
| KK | 212 (20 ug/mL) | 1.1 | 3.4 | 3 |

Notes:

No anti-proliferative properties

TABLE B-1

| | MTT50 | Number of |
|---|---|---|
| Cell line | uM | Experiments |
| Leukemic | | |
| AML-3 | >134 (25 ug/mL) | 6 |
| KK | >134 (25 ug/mL) | 6 |
| Normal | | |
| WI38 | >134(25 ug/mL) | 6 |
| Normal bone marrow | >134 (25 ug/mL) | 3 |

Table B-2

Trypan Blue Exclusion Assays n=3

TABLE B-3

| | Flow Cytometry | | | | | |
|---|---|---|---|---|---|---|
| | Fixed PI | | | | | |
| | | | | Acetone ctrl | | |
| | Dose | % pre- | % | | % | Number of |
| Cell line | (uM) | G1 | G2/M | % pre-G1 | G2/M | Experiments |

Leukemic

| | | | | | | |
|---|---|---|---|---|---|---|
| AML-3 | 107 (20 | 0.3 | 13.4 | 0.3 | 13.9 | 3 |
| | ug/mL) | | | | | |
| KK | 107 (20 | 1.1 | | 3.4 | | 3 |
| | ug/mL) | | | | | |

TABLE B-4

| | Flow Cytometry | | | |
|---|---|---|---|---|
| | TUNEL | | | |
| | Dose | TUNEL | Acetone | Number of |
| Cell line | (uM) | FITC +ve | TUNEL FITC +ve | Experiments |
| Leukemic | | | | |
| AML-3 | 107 (20 | 3.85 | 3.1 | 2 |
| | ug/mL) | | | |

Notes:
No anti-proliferative activity

TABLE C-1

| Cell | MTT50 | Number of |
|---|---|---|
| line | uM | Experiments |
| Leukemic | | |
| AML-2 | 13.2 | 2 |
| AML-3 | 16.9 | 6 |
| NB-4 | 11.7 | 2 |
| KK | 31.4 | 6 |
| B1 | 13.0 | 12 |
| W1 | 8.7 | 12 |
| Normal | | |
| WI38 | 89.0 | 16 |
| Normal bone marrow | 20.9 | 3 |

TABLE C-2

| | Flow Cytometry | | | | | |
|---|---|---|---|---|---|---|
| | Fixed PI | | | | | |
| | | | | Acetone ctrl | | |
| | Dose | % pre- | % | | % | Number of |
| Cell line | (uM) | G1 | G2/M | % pre-G1 | G2/M | Experiments |

Leukemic

| | | | | | | |
|---|---|---|---|---|---|---|
| AML-3 | 20.2 (10 | 10.7 | 11.1 | 0.3 | 13.9 | 3 |
| | ug/mL) | | | | | |
| KK | 40.3 (20 | 20.6 | | 3.4 | | 3 |
| | ug/mL) | | | | | |

TABLE C-3

| | Flow Cytometry | | | |
|---|---|---|---|---|
| | TUNEL | | | |
| | Dose | TUNEL | Acetone | Number of |
| Cell line | (uM) | FITC +ve | TUNEL FITC +ve | Experiments |
| Leukemic | | | | |
| AML-3 | 20.2 (10 | 22.4 | 3.1 | 2 |
| | ug/mL) | | | |

Notes:
Anti-proliferative activity

TABLE D-1

| Cell | MTT50 | Number of |
|---|---|---|
| line | uM | Experiments |
| Leukemic | | |
| AML-3 | 10.1 | 6 |
| KK | 12.8 | 6 |
| Normal | | |
| WI38 | 45.3 | 6 |
| Normal bone marrow | 19.1 | 3 |

TABLE D-2

| | Flow Cytometry | | | |
|---|---|---|---|---|
| | Fixed PI | | | |
| Cell | Dose | | Acetone ctrl | Number of |
| line | (uM) | % pre-G1 | % pre-G1 | Experiments |
| Leukemic | | | | |
| AML-3 | 91.6 (20 ug/mL) | 24.1 | 0.8 | 13 |
| KK | 91.6 (20 ug/mL) | 33.9 | 3.4 | 3 |

Notes:
Anti-proliferative activity

TABLE E-1

| Cell | MTT50 | Number of |
|---|---|---|
| line | uM | Experiments |
| Leukemic | | |
| AML-2 | 15.6 | 2 |
| AML-3 | 15.4 | 6 |
| NB-4 | 25.4 | 2 |
| KK | 17.5 | 6 |
| B1 | 4.3 | 2 |
| G2 | 3.6 | 2 |
| Normal | | |
| WI38 | 45.2 | 6 |
| Normal bone marrow | 21.9 | 3 |

TABLE E-2

| | Flow Cytometry | | | |
|---|---|---|---|---|
| | Fixed PI | | | |
| Cell | Dose | | Acetone ctrl | Number of |
| line | (uM) | % pre-G1 | % pre-G1 | Experiments |
| Leukemic | | | | |
| AML-3 | 85.3 (20 ug/mL) | 24.5 | 0.8 | 3 |
| KK | 85.3 (20 ug/mL) | 34.7 | 3.4 | 3 |

Notes:
Anti-proliferative activity

TABLE F-1

| Cell | MTT50 | Number of |
|---|---|---|
| line | uM | Experiments |
| Leukemic | | |
| AML-2 | 37.6 | 2 |
| AML-3 | 44.0 | 6 |
| KK | 93.4 | 6 |
| Normal | | |
| WI38 | >100 | 6 |
| | (25 ug/mL) | |
| Normal bone marrow | 115.8 | 3 |

Table F-1

Trypan Blue Exclusion Assays n=3

TABLE F-3

| | Flow Cytometry | | | | | |
|---|---|---|---|---|---|---|
| | Fixed PI | | | | | |
| | | | | Acetone ctrl | | |
| | Dose | % pre- | % | | % | Number of |
| Cell line | (uM) | G1 | G2/M | % pre-G1 | G2/M | Experiments |

Leukemic

| | | | | | | |
|---|---|---|---|---|---|---|
| AML-3 | 45.8 (10 | 5.1 | 34.1 | 0.3 | 13.9 | 3 |
| | ug/mL) | | | | | |
| KK | 91.6 (20 | 3.8 | | 3.4 | | 3 |
| | ug/mL) | | | | | |

Normal

| | | | | | | |
|---|---|---|---|---|---|---|
| WI38 | 50 (229 | 0.1 | 17.8 | 0.1 | 14.0 | 2 |
| | ug/mL) | | | | | |

TABLE F-4

| | Flow Cytometry | | | |
|---|---|---|---|---|
| | TUNEL | | | |
| | | | Acetone | |
| | Dose | TUNEL | TUNEL | Number of |
| Cell line | (uM) | FITC +ve | FITC +ve | Experiments |
| Leukemic | | | | |
| AML-3 | 45.8 (10 ug/mL) | 31.9 | 3.1 | 2 |

Notes:

Anti-proliferative Activity

TABLE G-1

| | | |
|---|---|---|
| | MTT50 | Number of |
| Cell line | uM | Experiments |

Leukemic

| | | |
|---|---|---|
| AML-3 | 114 (>25 ug/mL) | 6 |
| KK | 114 (>25 ug/mL) | 6 |

Normal

| | | |
|---|---|---|
| WI38 | 114 (>25 ug/mL) | 6 |
| Normal bone marrow | 114 (>25 ug/mL) | 3 |

Table G-1

Trypan Blue Exclusion Assays n=3

TABLE G-3

| | Flow Cytometry | | | | | |
|---|---|---|---|---|---|---|
| | Fixed PI | | | | | |
| | | | | Acetone ctrl | | |
| | Dose | % pre- | % | | % | Number of |
| Cell line | (uM) | G1 | G2/M | % pre-G1 | G2/M | Experiments |

Leukemic

| | | | | | | |
|---|---|---|---|---|---|---|
| AML-3 | 45.8 (10 | 0.3 | 13.5 | 0.3 | 13.9 | 3 |
| | ug/mL) | | | | | |
| KK | 91.6 (20 | 3.8 | | 3.4 | | 3 |
| | ug/mL) | | | | | |

TABLE G-4

| | Flow Cytometry | | | |
|---|---|---|---|---|
| | TUNEL | | | |
| | | | Acetone | |
| | | TUNEL | TUNEL | Number of |
| Cell line | Dose (uM) | FITC +ve | FITC +ve | Experiments |

Leukemic

| | | | | |
|---|---|---|---|---|
| AML-3 | 45.8 (10 ug/mL) | 3.0 | 3.1 | 2 |

Notes:
No anti-proliferative activity

TABLE H-1

| | | |
|---|---|---|
| | MTT50 | Number of |
| Cell line | uM | Experiments |

Leukemic

| | | |
|---|---|---|
| AML-3 | 93.4 | 6 |
| KK | 110.4 | 6 |

Normal

| | | |
|---|---|---|
| WI38 | >164 (25 ug/mL) | 6 |
| Normal bone marrow | 366.2 | 3 |

Table H-2

Trypan Blue Exclusion Assays n=3

TABLE H-3

| | Flow Cytometry | | | |
|---|---|---|---|---|
| | Fixed PI | | | |
| | | | Acetone ctrl | Number of |
| Cell line | Dose (uM) | % pre-G1 | % pre-G1 | Experiments |

Leukemic

| | | | | |
|---|---|---|---|---|
| AML-3 | 65.7 (10 ug/mL) | 3.6 | 0.3 | 3 |
| KK | 133.4 (20 ug/mL) | 5.9 | 3.4 | 3 |

Normal

| | | | | |
|---|---|---|---|---|
| WI38 | 985 (150 ug/mL) | 0.7 | 0.1 | 2 |

TABLE H-4

| | Flow Cytometry | | | |
|---|---|---|---|---|
| | TUNEL | | | |
| | | | Acetone | |
| | | TUNEL | TUNEL | Number of |
| Cell line | Dose (uM) | FITC +ve | FITC +ve | Experiments |

Leukemic

| | | | | |
|---|---|---|---|---|
| AML-3 | 65.7 (10 ug/mL) | 10.2 | 3.1 | 2 |

Notes:
Anti-proliferative activity

TABLE I-1

| | | |
|---|---|---|
| | MTT50 | Number of |
| Cell line | uM | Experiments |

Leukemic

| | | |
|---|---|---|
| AML-3 | 31.5 | 6 |
| KK | 31.5 | 6 |

Normal

| | | |
|---|---|---|
| WI38 | 76.9 | 6 |
| Normal bone marrow | 32.8 | 3 |

Table I-2

Trypan Blue Exclusion Assays n=3

TABLE I-3

| | Flow Cytometry | | | |
|---|---|---|---|---|
| | Fixed PI | | | |
| | | | Acetone ctrl | Number of |
| Cell line | Dose (uM) | % pre-G1 | % pre-G1 | Experiments |

Leukemic

| | | | | |
|---|---|---|---|---|
| AML-3 | 232.8 (5 ug/mL) | 23.6 | 0.3 | 3 |
| KK | 65.7 (10 ug/mL) | 48.4 | 3.4 | 3 |

TABLE I-4

| | Flow Cytometry | | | |
|---|---|---|---|---|
| | TUNEL | | | |
| | | | Acetone | |
| | | TUNEL | TUNEL | Number of |
| Cell line | Dose (uM) | FITC +ve | FITC +ve | Experiments |

Leukemic

| | | | | |
|---|---|---|---|---|
| AML-3 | 32.8 (5 ug/mL) | 57.3 | 3.1 | 2 |

Notes:
Anti-proliferative activity

TABLE J-1

| | | |
|---|---|---|
| | MTT50 | Number of |
| Cell line | uM | Experiments |

Leukemic

| | | |
|---|---|---|
| AML-3 | >162 (25 ug/mL) | 6 |
| KK | >162 (25 ug/mL) | 6 |

Normal

| | | |
|---|---|---|
| WI38 | >162 (25 ug/mL) | 6 |
| Normal bone marrow | >162 (25 ug/mL) | 3 |

TABLE J-2

| | Flow Cytometry | | | |
|---|---|---|---|---|
| | Fixed PI | | | |
| | | | Acetone ctrl | Number of |
| Cell line | Dose (uM) | % pre-G1 | % pre-G1 | Experiments |

Leukemic

| | | | | |
|---|---|---|---|---|
| AML-3 | 129.6 (ug/mL) | 0.2 | 0.3 | 3 |
| KK | 129.6 (ug/mL) | 2.6 | 3.4 | 3 |

Notes:
No anti-proliferative activity

TABLE K-1

| | MTT50 | Number of |
|---|---|---|
| Cell line | (uM) | repeats |
| Leukemic | | |
| B1 | 6.07 | 4 |
| G2 | 6.61 | |
| KK | 6.6 | |
| NB-4 | 6.36 | |
| AML-3 | 4.6 | |
| Normal | | |
| WI38 | 19.01 | 4 |

TABLE K-2

| | MTT50 | Number of |
|---|---|---|
| Cell line | (uM) | repeats |
| Leukemic | | |
| G2 | 5.8 | 1 |
| NB-4 | 7.8 | |
| Normal | | |
| Normal bone | 22.04 | |
| marrow | | |

TABLE L-1

| | MTT50 | Number of |
|---|---|---|
| Cell line | (uM) | Repeats |
| Leukemic | | |
| B1 | 6.34 | |
| W1 | 7.26 | |
| G2 | 3.8 | |
| KK | 6.3 | |
| NB-4 | 15.09 | |
| AML-3 | 7.46 | |
| Normal | | |
| WI38 | 19.69 | |

TABLE L-2

| | MTT50 | Number of |
|---|---|---|
| Cell line | (uM) | Repeats |
| Leukemic | | |
| NB-4 | 32.64 | 1 |
| Normal | | |
| WI38 | 7.9 | |
| Normal bone | 44.21 | |
| marrow | | |

TABLE M-1

| | MTT50 | Number of |
|---|---|---|
| Cell line | (uM) | Repeats |
| Leukemic | | |
| B1 | 12.73 | 6 |
| W1 | 4.8 | |
| G2 | 11.28 | |
| KK | 10.48 | |
| NB-4 | 21.23 | |
| AML-3 | 15.03 | |
| Normal | | |
| WI38 | 13.85 | |

TABLE M-2

| | MTT50 | Number of |
|---|---|---|
| Cell line | (uM) | Repeats |
| Leukemic | | |
| AML-3 | 8.53 | 2 |
| Normal | | |
| WI38 | 17.45 | |
| Normal bone | 22.61 | |
| marrow | | |

TABLE M-3

| | Flow Cytometry | | |
|---|---|---|---|
| | Fixed PI | | |
| | | | Acetone ctrl |
| Cell line | Dose (um) | % pre-G1 | % pre-G1 |
| Leukemic | | | |
| B1 | 20 | 70 | 2 |
| | 40 | 76 | |
| W1 | 20 | 9 | 0.3 |
| | 40 | 28 | |
| G2 | 20 | 10 | 1 |
| | 40 | 43 | |
| NBA | 20 | 16 | 6 |
| | 40 | 60 | |
| AML-3 | 20 | 34 | 1 |
| | 40 | 56 | |
| Normal | | | |
| WI38 | 120 | 119 | 0.2 |
| | 40 | 16 | |

TABLE N-1

| | MTT50 | Number of |
|---|---|---|
| Cell line | (uM) | Repeats |
| Leukemic | | |
| B1 | 94.39 | 6 |
| KK | 65.24 | |
| NB-4 | 60.78 | |
| AML-3 | 51.08 | |
| Normal | | |
| WI38 | >100 | |

TABLE N-2

| | MTT50 | Number of |
|---|---|---|
| Cell line | (uM) | Repeats |
| Leukemic | | |
| AML-3 | 73.36 | 1 |
| Normal | | |
| WI38 | >100 | |
| Normal bone | >100 | |
| marrow | | |

TABLE N-3

| | Flow Cytometry | | | |
|---|---|---|---|---|
| | Fixed PI | | | |
| | | | Acetone ctrl | % G2 |
| Cell line | Dose (uM) | % Pre-G1 | % pre-G1 | increase |
| Leukemic | | | | |
| B1 | 50 | 117 | 2 | +2 |
| | 150 | 35 | | −1 |
| W1 | 50 | 22 | 7 | +10 |
| | 150 | 33 | | |
| G2 | 50 | 16 | 2 | +8 |
| | 150 | 29 | | +8 |
| KK | 50 | 13 | 7 | +4 |
| | 150 | 10 | | +5 |
| AML-3 | 50 | 17 | 2 | +17 |
| | 150 | 35 | | +14 |
| Normal | | | | |
| WI38 | 50 | 2 | 1 | +7 |
| | 150 | 4 | | +25 |

TABLE N-4

| | Flow Cytometry | | |
|---|---|---|---|
| | TUNEL | | |
| | | | Acetone ctrl |
| | | % TUNEL | (% TUNEL |
| Cell line | Dose (uM) | +ve | +ve) |
| Leukemic | | | |
| B1 | 50 | 49 | 1 |
| | 100 | 44 | |
| G2 | 50 | 18 | 2 |
| | 100 | 19 | |
| NB-4 | 50 | 17 | 8 |
| | 100 | 19 | |
| Normal | | | |
| WI38 | 50 | 0.7 | 0.2 |
| | 100 | 2 | |

TABLE O-1

| | MTT50 | Number of |
|---|---|---|
| Cell line | (uM) | Repeats |
| Leukemic | | |
| B1 | 57.43 | 4 |
| KK | 96.9 | |
| NB-4 | 62.89 | |
| AML-3 | 55.75 | |
| Normal | | |
| WI38 | 73.87 | |

TABLE 0-2

| | | Number of |
|---|---|---|
| | MTT50 | Repeats |
| Cell line | (uM) | (%) |
| Leukemic | | |
| AML-3 | 44.09 | 1 |
| Normal | | |
| Normal Bone | >100 | |
| Marrow | | |

TABLE P-1

| | MTT50 | Number of |
|---|---|---|
| Cell line | (uM) | Repeats |
| Leukemic | | |
| B1 | 94.38 | 6 |
| KK | 65.24 | |
| NB-4 | 60.78 | |
| AML-3 | 51.08 | |
| Normal | | |
| WI38 | >100 | |

TABLE P-2

| | MTT50 | Number of |
|---|---|---|
| Cell line | (uM) | Repeats |
| Normal | | |
| WI38 | >100 | 1 |
| Normal bone | >100 | |
| marrow | | |

TABLE P-3

| | Flow Cytometry | | |
|---|---|---|---|
| | Fixed PI | | |
| | | | Acetone ctrl |
| Cell line | Dose (uM) | % pre-G1 | % pre-G1 |
| Leukemic | | | |
| B1 | 50 | 52 | 2 |
| | 150 | 80 | |
| W1 | 50 | 14 | 0.3 |
| | 150 | 55 | |
| G2 | 50 | 6 | 1 |
| | 150 | 63 | |
| NB-4 | 50 | 18 | 6 |
| | 150 | 75 | |
| AML-3 | 50 | 7 | 1 |
| | 150 | 66 | |
| Normal | | | |
| WI38 | 50 | 2 | 0.2 |
| | 150 | 52 | |

TABLE P-4

| | Flow Cytometry | | |
|---|---|---|---|
| | TUNEL | | |
| | | | Acetone ctrl |
| | | % TUNEL | (% TUNEL |
| Cell line | Dose (uM) | +ve | +ve) |
| Leukemic | | | |
| B1 | 50 | 39 | 1 |
| | 100 | 67 | |
| G2 | 50 | 17 | 2 |
| | 100 | 53 | |
| KK | 50 | 47 | 1 |
| | 100 | 78 | |
| NB-4 | 50 | 24 | 8 |
| | 100 | 0.2 | |
| Normal | | | |
| WI38 | 50 | 0.70 | 0.2 |
| | 100 | 1.7 | |

TABLE Q-1

| | MTT50 | Number of |
|---|---|---|
| Cell line | (uM) | repeats |
| Leukemic | | |
| B1 | 14.28 | 6 |
| W1 | 13.03 | |
| G2 | 14.68 | |
| KK | 23.69 | |
| AML-3 | 22.5 | |
| Normal | | |
| WI38 | >100 | |

TABLE Q-2

| | MTT50 | Number of |
|---|---|---|
| Cell line | (uM) | repeats |
| Normal | | |
| WI38 | >100 | 1 |
| Normal bone | >100 | |
| marrow | | |

TABLE Q-3

| | MTT50 | Number of |
|---|---|---|
| Cell line | (uM) | repeats |
| Normal | | |
| WI38 | >100 | 1 |
| Breast | | 1 |
| MDA-231 | 68.87 | |
| SK-BR-3 | 59 | |
| ZR-751 | 52 | |
| Prostate | | 1 |
| DU145 | 65.43 | |
| Melanoma | | 1 |
| WM9 | 41 | |
| WM983 | 46.98 | |
| WM793 | 47 | |
| 1232 | 56 | |

TABLE Q-4

| | Flow Cytometry | | | |
|---|---|---|---|---|
| | Fixed PI | | | |
| | | | Acetone ctrl | % G2 |
| Cell line | Dose (uM) | % pre-G1 | % pre-G1 | increase |

Leukemic

| | | | | |
|---|---|---|---|---|
| B1 | 20 | 5 | 2 | 0 |
| | 40 | 20 | | −3 |
| W1 | 20 | 20 | 7 | +4 |
| | 40 | 49 | | +12 |
| G2 | 20 | 31 | 2 | −1 |
| | 40 | 55 | | −3 |
| KK | 20 | 15 | 7 | |
| | 40 | 43 | | −4 |
| AML-3 | 20 | 10 | 2 | −5 |
| | 40 | 42 | | −9 |
| Normal | | | | |
| WI38 | 20 | 1 | 1 | +3 |
| | 40 | 1 | | +10 |

TABLE Q-5

| | Flow Cytometry | | |
|---|---|---|---|
| | TUNEL | | |
| | | | Acetone ctrl |
| Cell line | Dose (uM) | % TUNEL +ve | (% TUNEL +ve) |
| Leukemic | | | |
| B1 | 25 | 32 | 1 |
| | 50 | 78 | |
| G2 | 25 | 23 | 2 |
| | 50 | 58 | |
| KK | 25 | 43 | 1 |
| | 50 | 61 | |
| NB-4 | 50 | 16 | 8 |
| Normal | | | |
| WI38 | 25 | 4 | 0.2 |
| | 50 | 0.2 | |

Discussion

Initially, the efficacy of ten synthetic OSCs was explored for their antiproliferative activity against mammalian cells. This was expanded to include seven more compounds. On the basis of activity (MTT assays) the OSCs were separable into three distinct groups; Group I (compounds A, B, G, J), Group II (compounds F, H) and Group III (compounds C, D, E, I). The trypan blue exclusion results, in combination with the MTT activity results indicate that compounds C, D, E, F, H and I were cytotoxic to the leukemic cells in a dose-dependent manner. Exposure to compounds F and H led to an accumulation of leukemic cells in the G2/M phase of the cell cycle prior to the cells undergoing apoptosis. By contrast, compounds C, D, E and I trigger the leukemic cells to undergo apoptosis in all the phases of the cell cycle as determined by fixed PI and TUNEL. Interestingly, the viability of non-transformed human WI38 fibroblasts was not affected following exposure to compounds A, B, G, J, F and H yet decreased in response to compounds C, D, E and I. The common structural feature of all the active antileukemic OSCs analyzed in this study was the presence of a disulphide. Indeed, many biologically active natural products of the genus allium are disulphides or closely related thiosulfinates or thiosulfonates (Block, E: The organosulfur chemistry of the genus Allium—implications for the organic chemistry of sulfur. Angew. Chem. Int. Ed. Engl. 31: 1135-1178, 1992). Clearly in this study, however, the sulfone disulphide F and disulphide ester H exhibit specificity towards transformed cell lines.

A handful of investigators have explored the effects of naturally-occurring OSCs, such as S-allylmercaptocysteine (SAMC) or diallyl disulphide (DADS) on the growth of tumor cell lines. Sigounas et al. (Sigounas, G, Hooker, J L, Li, W, Anagnostou, A and Steiner, M: S-allylmercaptocysteine, a stable thioallyl compound, induces apoptosis in erythroleukemia cell lines. Nutr Cancer 28: 153-9, 1997, Sigounas, G, Hooker, J, Anagnostou, A and Steiner, M: S-allylmercaptocysteine inhibits cell proliferation and reduces the viability of erythroleukemia, breast, and prostate cancer cell lines. Nutr Cancer 27: 186-91, 1997) have shown that SAMC reduces viability of erythroleukemia, breast and prostate cancer cell lines. The effects of compounds F and H appear to be similar to SAMC and DADS. Both SAMC and DADS have been shown to growth arrest cells in the G2/M phase of the cell cycle prior to apoptosis in a time and dose dependent manner (Sigounas, G, Hooker, J L, Li, W, Anagnostou, A and Steiner, M: S-allylmercaptocysteine, a stable thioallyl compound, induces apoptosis in erythroleukemia cell lines. Nutr Cancer 28: 153-9, 1997, Sundaram, S G and Milner, J A: Diallyl disulphide inhibits the proliferation of human tumor cells in culture. Biochim Biophys Acta 1315: 15-20, 1996, Sundaram, S G and Milner, J A: Diallyl disulphide induces apoptosis of human colon tumor cells. Carcinogenesis 17: 669-73, 1996, Knowles, L M and Milner, J A: Depressed p34cdc2 kinase activity and G2/M phase arrest induced by diallyl disulphide in HCT-15 cells. Nutr Cancer 30: 169-74, 1998). However, unlike compound F and H, SAMC does not appear to be tumor-specific as it has been shown to inhibit proliferation of non-transformed cells (Sigounas, G, Hooker, J L, Li, W. Anagnostou, A and Steiner, M: S-allylmercaptocysteine, a stable thioallyl compound, induces apoptosis in erythroleukemia cell lines. Nutr Cancer 28: 153-9, 1997). Compared to these natural compounds, Group II and Group III OSCs in this study were approximately 2 and 10 fold more potent, respectively, suggesting the specific activity of natural OSCs can be increased by structure-activity analysis of synthetic derivatives.

It appears that the structural criteria of OSCs for their antifungal and antitumor activities are separable and distinct. Comparison of the antifungal (Baerlocher, F J, Langler, R F, Frederiksen, M U, Georges, N M and Witherell, R D: Structure-activity relationships for selected sulfur-rich antifungal compounds. Aust. J. Chem. 52: 167-172, 1999) and antileukemic activity of the synthetic OSCs reveals compounds A, B, G and J do not possess antiproliferative activity on either fungal or mammalian cells. By contrast, compounds C, E and I possess antifungal and anti-mammalian cell activity, which is consistent with the notion that disulphides are general toxins (Rice, W G, Turpin, J A, Schaeffer, C A, Graham, L, Clanton, D, Buckheit, R W, Jr., Zaharevitz, D, Summers, M F, Wallqvist, A and Covell, D G: Evaluation of selected chemotypes in coupled cellular and molecular target-based screens identifies novel HIV-1 zinc finger inhibitors. J Med Chem 39: 3606-16, 1996). Finally, compounds D, F and H do not possess antifungal activity but can inhibit mammalian cell growth. Moreover, compound F and H are distinct from compound D as these compounds possess tumor-specific activity. Unlike other natural products currently being explored as therapeutics, these compounds are relatively simple and inexpensive to synthesize. The discussion under the heading “Summary of Results” describes these results as well as the results for compounds K to Q.

Preparatory Methods

The methods described herein encompass both novel and known methods of preparation. The preparation of novel compound 4 in Table 6 represents a novel method. Compound Q is a novel compound and its method of preparation is also novel. The method for preparing compound 2 in Table 7 is new, but compound 3 is made by a known method. The same is true for compound 4 of Table 7 and for compound P. The following description provides an overview of various aspects of the methods described herein. It is believed that the person skilled in the art could readily apply the various methods for preparing the compounds of this invention to produce additional compounds having the same basic structure. The additional detail found in the specific methods described provides instruction for the detail of such methods. This is particularly true for any group of compounds or specific compound mentioned herein, the preparation for which is not described.

From the beginning of this program to make biologically active organosulfur compounds, α-substituted disulphides have been targeted for synthesis. Initially, singly α-substituted dimethyl disulphides i.e. 1 were chosen for construction.

CH₃SSCH₂W 1

A) Conditions for disproportionations in dimethyl disulphide which afford unsymmetrical methyl disulphides smoothly have been optimized. This chemistry affords methyl disulphides even when reactions proceed through the metastable intermediates: RSCH₂S⁻ (see eq. [1] for an example).

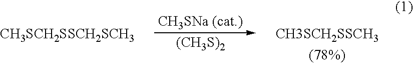

B) Well-known, base-catalyzed condensations of sulfenyl chlorides and mercaptans have been exploited for the construction of unsymmetrical disulphides. Symmetrical disulphides have traditionally been used to make sulfenyl chlorides, so that only one sulfenyl chloride is produced (see eq. [2]).

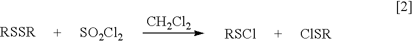

In a number of cases, symmetrical disulphides (e.g. (p-O₂N(C₆H₄)S)₂ or (CH₃OC(O)CH₂S)₂) will not react with our chlorinating agent. It has now been established that the corresponding methyl disulphides cleave smoothly with SO₂Cl₂/CH₂Cl₂ (see eq.[3] for an example).

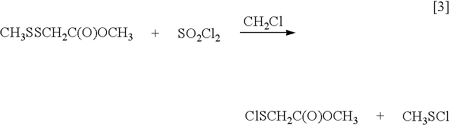

Methanesulfenyl chloride is very volatile and is completely removed when the solvent is evaporated. Hence, we have established that unsymmetrical methyl disulphides are useful precursors for the preparation of homogeneous sulfenyl chlorides.

C). Now described are new transition metal oxidations of dialkyl disulphides which furnish, the virtually unknown α-ester disuiphides in which the ester group is attached to the disulphide framework by an oxygen atom (see eq.[4] for an example).

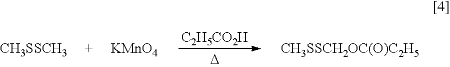

These α-ester disuiphides show, inter alia, antifungal and antileukemic properties.

D) It has been established that α-ester disulphides serve as effective precursors for the preparation of α-sulfonyl disulphides which also show antifungal and antileukemic properties (see eq.[5] for an example).

CH₃SSCH₂OC(O)C₂H₅+p-CH₃(C₆H₄)SO₂Na-->CH₃SSCH₂SO₂(CH₆H₄)(CH₃-p (5]

E) In a powerful conjunction of B) and D), the sulfenyl chloride ester 2 has been prepared.

ClSCH₂OC(O)C₂H₅ 2

This versatile intermediate permits nucleophilic attack at both S and at C. It is a synthetic equivalent for 3.

⁺S—CH₂⁺ 3

3 permits general access to unsymmetrical disulphides, through α-ester disulphides (see eq.[6]).

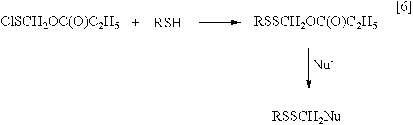

Infrared spectra were recorded on a Perkin-Elmer 710B grating spectrophotometer for chloroform solutions unless otherwise specified. ¹H n.m.r. spectra (60 MHz) were obtained on a Varian EM360L instrument. ¹H n.m.r. (270 MHz) and ¹³C n.m.r. spectra were obtained on a JEOL JNM-GSX 270 Fourier-transform n.m.r. system. Unless otherwise specified, all n.m.r. spectra were obtained for (D)chloroform solutions with tetramethylsilane as internal standard. Mass spectra were obtained on a Hewlett-Packard 55988A g.l.c./m.s. system. Melting points were determined on a Gallenkamp MFB-595 capillary melting point apparatus and are uncorrected.

Preparation of Compounds of Table 3

Previously Prepared Compounds

Compounds (4) and (6) were prepared as described in Ahern, T. P., Hennigar,. T. L., MacDonald, J. A., Morrison, H. G., Langler, R. F., Satyanarayana, S., and Zawarotko, M. J., Aust. J. Chem., 1997, 50, 683 and compound (8) was prepared as described in Ahern, T. P., Langler, R. F., and McNeil, R. L., Can. J. Chem., 1980, 58, 1996. Compound (13) was prepared as described earlier (Georges, N. M. Johnson, M. D., Langler, R. F., and Verma, S. D., Sulfur Lett. 22, 141 (1999). Compounds (5) (Robson, P., Speakman, P. H. R., and Stewart, D. G., J. Chem. Soc. C, 1968, 2180, Bohme, H., and Heller, P., Chem. Ber., 1953, 86, 785) and (7) (Dubs, P., and Stuessi, R., Helv. Chim. Acta, 1978, 61, 2351) have been prepared by earlier workers.

Preparation of 2,4-Dithiapentane 2,2-Dioxide (5)

(A) A solution of 2,4-dithiapentane (0.98 g, 9.0 mmol) and hydrogen peroxide (1.03 g, 30%) in 1,4-dioxan (24 ml) was refluxed behind a safety shield for 0.5 h and the solvent evaporated.

(B) Potassium permanganate (0.74 g, 4.7 mmol) was covered with water (4.2 ml) and tetrahydrofuran (17 ml). Crude product from part (A) was dissolved in water (7 ml) and tetrahydrofuran (28 ml) and added to the reaction mixture. The reaction mixture was stirred at ambient temperature for 1 h and filtered through a Celite filter pad. The filtered solution was added to solid sodium thiosulfate (35 g) and the mixture stirred for 0.5 h. The solid was filtered off and the organic solvent evaporated affording a wet residue. The residue was extracted with chloroform (three 100 ml aliquots). The organic layers were combined, dried (MgSO₄), filtered and concentrated. The crude product was chromatographed on silica gel (70 g) employing chloroform elution (50 ml fractions). Fractions 14-16 were combined and concentrated affording clean 2,4-dithiapentane 2,2-dioxide (5) (0.13 g, 0.9 mmol, 10%) as an oil. l.r. 1310, 1160 cm⁻¹. ¹H n.m.r. (60 MHz) δ2.43, s, 3H; 3.06, s, 3H; 3-85, s, 2H. m/z 140 (9%, M⁺), 61 (100).

Preparation of 2,3,5-Trithiahexane (7)

Sodium metal (0.163 g, 7.0 mmol) was dissolved in methanol (10 ml), the solvent evaporated and the sodium methoxide dried in vacuum. Dimethyl sulfoxide (Me₂SO)(3 ml) was added and the resultant suspension stirred vigorously for 5 h.

Dimethyl disulphide (3.7 g, 38.8 mmol), 3,5-dithiahexan-2one (Ahern, T. P., Haley, M. F., Langler, R. F. and Trenholm, J. E. Can. J. Chem., 1984, 62, 610) (1.0 g, 7.7 mmol) and Me₂SO (2 ml) were added. The reaction mixture was stirred at ambient temperature for 2 days. Hydrochloric acid (2.5%, 100 ml) was added and the resultant mixture extracted with diethyl ether (three 100 ml aliquots). The combined organic layers were concentrated and the extraction procedure was repeated. The combined organic layers were dried (MgS04) and filtered, and the solvent was evaporated.

The crude product was chromatographed on silica gel (10 g) employing light petroleum (10 ml fractions) for elution. Fractions 3-7 were combined and the residue was distilled furnishing 2,3,5-trithiahexane (7) (0.45 g, 3.2 mmol, 42%), b.p. 106° C./18 Torr. ¹H n.m.r. (270 MHz) δ2.23, s, 3H; 2.50, s, 3H; 3.85, s, 2H. ¹³C n.m.r. δ15-15, 23.42, 44.22. m/z 140 (26%, M⁺), 61 (100).

Preparation of 2,4,5-Trithiaheptane 2,2-Dioxide (9)

Ethanethiol (1.4 g, 22.4 mmol) was dissolved in dry pyridine (50 ml) and CH₃SO₂CC1₂SOCH₃ (Ahem, T. P., Langler, R. F., and McNeil, R. L., Can. J. Chem., 1980 58, 1996) (1.2 g, 4.5 mmol) added. The reaction mixture was stirred at ambient temperature for 24 h. Chloroform (200 ml) was added and the resultant mixture washed with 5% hydrochloric acid (100 ml aliquots) until the aqueous layer remained acidic. The organic layer was dried (MgSO₄), filtered and concentrated. The residue was chromatographed on silica gel (150 g) employing chloroform elution (100 ml fractions). Fractions 8-11 were combined and concentrated affording clean sulfone disulphide (9) (0.45 g, 2.4 mmol, 53%). Recrystallized 2,4,5-trithiaheptane 2,2-dioxide (methanol) had m.p. 34.6-36.3° C. (Found: C, 25.6; H, 5.4. C₄H₁₀O₂S₃ requires C, 25.8; H 5.4%). l.r. 1325, 1150 cm⁻¹. ¹H n.m.r. (270 MHz) δ1.37, t, 3H; 2.91, q, 2H; 3.04, s, 3H; 4.09, s, 2H. ¹³C n.m.r. δ14-17, 33.31, 39.21, 62.40. m/z 186 (13%, M⁺), 107 (67), 79 (100).

Preparation of 3,6-Dithiaheptan-2-one

Thioacetic S-acid (49.8 g, 655 mmol) and 4-chloro-2-thiabutane (Fong, H. O., Hardstaff, W. R., Kay, D. G., Langler, R. F., Morse, R. G., and Sandoval, D. N., Can. J. Chem., 1979, 57, 1206) (40.0 g, 363 mmol) were added to dry pyridine (550 ml), and the reaction mixture was refluxed for 2 h. Dichloromethane (2.7 litres) was added and the resultant mixture extracted with 5% hydrochloric acid (600 ml portions) until the aqueous layer remained acidic. The organic layer was dried (MgSO₄), filtered and the solvent distilled off at atmospheric pressure. The residue was rectified at reduced pressure affording 3,6-dithiaheptan-2-one (32.1 g, 214 mmol, 59%), b.p. 80° C./43 Torr. l.r. (liquid film) 1690 cm⁻¹. ¹H n.m.r. (270 MHz) δ2.17, s, 3H; 2.35, s, 3H; 2.67, t, 2H; 3.10, t, 2H. ¹³C n.m.r. δ15.37, 28.72, 30.65, 33.86. m/z 150 (9%, M⁺), 74 (55), 61 (40), 43 (100).

Preparation of 3-Thiabutane-1-thiol

3,6-Dithiaheptan-2-one (32.1 g, 214 mmol) was dissolved in methanol (900 ml) and sodium hydroxide (11.1 g, 277 mmol) in water (500 ml) added. The reaction mixture was stirred at ambient temperature for 1 h. Water (1 litre) and 10% hydrochloric acid (400 ml) were added. The resultant mixture was extracted with methylene chloride (five 800 ml aliquots). The bulk of the solvent was distilled off at atmospheric pressure. The residue was dissolved in methylene chloride (500 ml) and the resultant solution washed with 5% sodium hydroxide solution (three 200 ml portions). The aqueous layer was strongly acidified (concentrated hydrochloric acid) and extracted with dichloromethane (three 200 ml aliquots). The combined organic layers were dried (MgSO₄) and filtered, and the solvent was distilled off at atmospheric pressure. Crude 3-thiabutane-1-thiol was rectified at reduced pressure (10.8 g, 100 mmol, 47%), b.p. 120° C./160 Torn l.r. (liquid film) 2580 cm⁻¹. ¹H n.m.r. (270 MHz) δ1.75, t, 1H; 2.15, s, 3H; 2.73, s, 2H; 2.75, s, 2H. ¹³C n.m.r. δ15.28, 24.13, 38.14. m/z 108 (100%, M⁺), 61 (95).

Preparation of 2,3,6-Trithiaheptane

Me₂SO (50 ml) was added to powdered sodium hydroxide (2.6 g, 65 mmol) and the reaction mixture stirred to produce a homogeneous solution. 3-Thiabutane-l-thiol (3.0 g, 27 mmol) in Me₂SO (20 ml) was added to the reaction mixture which was stirred at room temperature for 5 min. Dimethyl disulphide (6.5 g, 69.1 mmol) in Me₂SO (30 ml) was added and the resultant mixture stirred at ambient temperature for 24 h. Hydrochloric acid (2.5%, 1 litre) was added and the resultant mixture washed with diethyl ether (three 1 litre aliquots). The combined organic layers were concentrated and the extraction procedure was repeated. The concentrate was covered with 2.5% sodium hydroxide solution (1 litre) and the resultant mixture extracted with diethyl ether (three 1 litre portions). The combined organic layers were dried (MgSO₄) and filtered, and the solvent was evaporated. The residue was distilled at reduced pressure giving 2,3,6-trithiaheptane as a colourless oil (2.4 g, 15.5 mmol, 24%), b.p. 80° C./2 Torr. ¹H n.m.r. (270 MHz) δ2.15, s, 3H; 2.43, s, 3H; 2-83, m, 2H; 2.92, m, 2H. ¹³C n.m.r δ15.54, 23.45, 33.54, 37.24. m/z 154 (1%, M⁺), 75 (100).

Preparation of 2,3,6-Trithiaheptane 6-Oxide

2,3,6-Trithiaheptane (1.0 g, 6.4 mmol) was dissolved in 1,4-dioxan (45 ml) and hydrogen peroxide (0.37 g, 30%) in 1,4-dioxan (5 ml) added. The reaction mixture was refluxed behind a safety shield for 30 min. The solvent was evaporated and the residue chromatographed on silica gel (100 g) employing chloroform (100 ml fractions) for elution. Fractions 9-18 were combined and concentrated affording clean 2,3,6-trithiaheptane 6-oxide as a colorless oil (0.54 g, 3.1 mmol. 48%). l.r. (liquid film) 1040 cm⁻¹. ¹H n.m.r. (270 MHz) δ2.42, s, 3H; 2.63, s, 3H; 3.03, m, 2H; 3.09, m, 211. ¹³C n.m.r. δ22.87, 29.55, 38.60, 53.49. m/z 106 (100%), 79 (95).

Preparation of 2,3,6-Trithiaheptane 6.6-Dioxide (10)

2,3,6-Trithiaheptane 6-oxide (0.54 g, 3.1 mmol) was dissolved in acetone (40 ml) and the reaction mixture cooled to 0° C. Anhydrous magnesium sulfate (3.8 g) in acetone (10 ml) was added and the reaction mixture stirred at ambient temperature. Potassium permanganate (0.50 g) was added in three portions at half-hour intervals. Upon completion of the addition, the reaction mixture was filtered through a Celite pad and the solvent evaporated. The residue was chromatographed on silica gel (50 g) employing 1:1 methylene chloride/light petroleum (50 ml fractions) for elution. Fractions 7-11 were combined and concentrated yielding clean (10) (0.51 g, 2.7 mmol, 87%). 2,3,6-Trithiaheptane 6,6-dioxide had b.p. 158-162° C./14 Torr (Found: C, 25.9; H, 5.6. C₄H₁₀0₂S₃ requires C, 25.8; H, 5.4%). l.r. (liquid film) 1310, 1145 cm⁻¹. ¹H n.m.r. (270 MHz) δ2.44, s, 3H; 2.99, s, 3H; 3.08, m, 2H; 3.43, m, 2H. ¹³C n.m.r. δ22.80, 28.67, 41.45, 54.17. m/z 186 (21%, M⁺), 106 (80), 79 (100).

Preparation of S-4-Thiapentyl Thioacetate 4,4Dioxide

(A) 4-Thiapentan-1-ol (Langler, R. F., Marini, Z. A., and Spalding, E. S., Can. J. Chem., 1979, 57, 3193. (5.3 g, 49.5 mmol) and triethylamine (4.9 g, 48.5 mmol) in dry pyridine (75 ml) were cooled with an ice/salt/water bath. Methanesulfonyl chloride (5.8 g, 50.8 mmol) was added dropwise over 15 min. The reaction mixture was stirred at ambient temperature for 3 days. Chloroform (100 ml) was added and the resultant mixture washed with 10% hydrochloric acid (50 ml portions) until the aqueous layer remained acidic. The organic layer was dried (MgSO₄), filtered and concentrated. The residue was rectified at reduced pressure affording impure sulphide methanesulfonate (3.30 g; b.p. 110-120° C./2 Torr).

(B) Impure sulphide methanesulfonate (3.8 g) from step (A) was dissolved in chloroform (75 ml), and the solution added dropwise to 10% sulfuric acid (104 ml). During the addition, solid potassium permanganate (13.7 g) was also added in small portions. Upon completion of the additions, the reaction mixture was stirred at ambient temperature for 2 days. The reaction mixture was cooled with an ice/water bath and sodium bisulfite added in small portions until the reaction mixture was decolorized. The layers were separated and the aqueous layer was extracted with chloroform (three 100 ml aliquots). The combined organic layers were concentrated affording impure sulfone methanesulfonate (3.7 g).

(C) Thioacetic S-acid (1.3 g) in dry pyridine (30 ml) was added to impure sulfone methanesulfonate (3.7 g) from step (B) and the reaction mixture stirred at room temperature for 2 days. Chloroform (200 ml) was added and the resultant mixture washed with 2.5% hydrochloric acid (100 ml aliquots) until the aqueous pH remained acidic. The organic layer was dried (MgSO₄), filtered and concentrated. Crude sulfone thioacetate was chromatographed on silica gel (250 g) employing 1:1 light petroleum/chloroform (100 ml fractions) for elution. Fractions 53-72 were combined and concentrated yielding clean S-4-thiapentyl thioacetate 4,4-dioxide (0.93 g, 4.7 mmol, 8% from 4-thiapentan-1-ol). Recrystallized (methanol) sulfone thioacetate had m.p. 61.8-62.7° C. (Found: C, 36.8; H, 6.1. C₆H₁₂0₃S₂ requires C, 36.7; H, 6.2%). l.r. (KBr) 1690, 1300, 1160, 1130 cm-⁻¹. ¹H n.m.r.(270 MHz) δ2.15, quin, 2H; 2.36, s, 3H; 2.92, s, 3H; 3.04, m, 4H. m/z 196 (6%, M⁺′), 116 (39), 43 (100).

Preparation of 4- Thiapentane-1-thiol 4,4-Dioxide

S-4-Thiapentyl thioacetate 4,4-dioxide (0.25 g, 1.2 mmol) was dissolved in methanol (25 ml) and a solution of sodium hydroxide (0.08 g, 2. 0 mmol) in water (125 ml) added. The reaction mixture was stirred at ambient temperature for 1 h. Water (25 ml) and 10% hydrochloric acid (6 ml) were added, and the resultant mixture was extracted with chloroform (four 50 ml aliquots). The combined organic layers ware dried (MgS0₄), filtered and concentrated giving 4-thiapentane-1-thiol 4,4-dioxide (0.13 g, 0.8 mmol, 67%). The thiol was subjected to a bulb-to-bulb distillation (bath: 200° C.; pressure: 1.5 Torr). l.r. (liquid film) 2600, 1310, 1145 cm¹. ¹H n.m.r. (270 MHz) δ1.45, t, 1H; 2.17, quin, 2H; 2.72, q, 2H; 2.95, s, 3H; 3.20, t, 2H. ¹³C n.m.r. δ23.21, 26.37, 40.88, 52.85. m/z 154 (36%, M⁻), 74 (100), 41 (76).

Preparation of 2,3,7-Trithiaoctane 7,7-Dioxide (II).

Me₂SO (2 ml) was added to powdered sodium hydroxide (0.10 g, 2.5 mmol) and the mixture stirred vigorously. 4-Thiapentane-l-thiol 4,4-dioxide (0.38 g, 2.5 mmol) in Me₂SO (4 ml) was added to the reaction mixture which was stirred for 5 min. Dimethyl disulphide (0.69 g, 7.3 mmol) in Me₂SO (4 ml) was added and the reaction mixture stirred at room temperature for 24 h. Hydrochloric acid (2.5%, 150 ml) was added and the resultant mixture washed with diethyl ether (three 100 ml aliquots). The combined organic layers were concentrated and the extraction procedure was repeated. Sodium hydroxide solution (2.5%, 150 ml) was added to the residue and the resultant mixture extracted with diethyl ether (three 100 ml portions). The combined organic layers were dried (MgS0₄) and filtered, and the solvent was evaporated. The crude product was chromatographed on silica gel (40 g) employing 1:1 methylene chloride/light petroleum (40 ml fractions) for elution. Fractions 12-24 were combined and concentrated furnishing clean sulfone disulphide (11) (0.15 g, 0.7 mmol, 30%). After recrystallization (methanol), 2,3,7-trithiaoctane 7,7-dioxide (11) had m.p. 34.8-35.3° C. (Found: C, 30.1; H 6.1. C₅H₂O₂S₃ requires C, 30.0; H 6.0%). l.r. 1305, 1140 cm⁻¹. ¹H n.m.r. (270 MHz) δ2.31, quin, 2H; 2.42, s, 3H; 2.84, t, 2H; 2.94, s, 3H; 3.17, t, 2H. ¹³C n.m.r. δ21.50, 23.00, 35.55, 40.85, 52.87. m/z 200 (13%, M⁺), 121 (100), 79 (76), 41 (78).

Preparation of Methyl 3,4Dithiapentanoate (12)

Sodium hydride (1.8 g, 76.5 mmol) was suspended in Me₂SO (30 ml) and methyl thioglycolate (5.2 g, 49.2 mmol) in Me₂SO (30 ml) was added. After 3 min, dimethyl disulphide (13.0 g, 138 mmol) in Me₂SO (90 ml) was added and the reaction mixture stirred at room temperature for 24 h. Hydrochloric acid (2.5%, 1 litre) was added to the reaction mixture and the resultant mixture extracted with diethyl ether (three 1 litre aliquots). The combined ether layers were concentrated and the extraction procedure was repeated. Sodium hydroxide solution (2.5%, 1 litre) was added to the residue and the resultant mixture extracted with diethyl ether (three 1 litre aliquots). The combined organic layers were dried (MgS0₄), filtered and concentrated. The residue was rectified at reduced pressure affording methyl 3,4-dithiapentanoate (12) (1.26 g, 8.2 mmol, 170%). The disulphide ester (12) had b.p. 108-110° C./18 Torr (Found: C, 31.4; H, 5.4 C₄H₈0₂S₂ requires C, 31.6; H, 5.3%). l.r. (liquid film) 1740 cm⁻¹. ¹H n.m.r. (270 MHz) δ2.47, s, 3H; 3.50, s, 2H; 3.77, s, 3H. ¹³C n.m.r. δ23.01, 40.67, 52.54, 170.20. m/z 152 (39%, M⁺), 93 (48),45 (100).

Pteparation of 3,4-Dithiapentan-1-ol

Me₂SO (30 ml) was added to sodium hydroxide (2.64 g, 65.4 mmol). A solution of 2-mercaptoethanol (5.0 g, 64.6 mmol) in Me_{z}SO (30 ml) was added and the reaction mixture stirred for 3 min. Dimethyl disulphide (18 g, 281 mmol) in Me₂SO (90 ml) was added and the reaction mixture stirred at ambient temperature for 1 week. Hydrochloric acid (2.5% 1 litre) was added and the resultant mixture washed with diethyl ether (three 1 litre aliquots). The combined organic layers were concentrated and the extraction procedure was repeated. The residue was dissolved in diethyl ether (1 litre) and extracted with 2.5% sodium hydroxide solution (1 litre). The organic layer was dried (MgS0₄) and filtered, and the solvent evaporated. The residue was rectified at reduced pressure furnishing clean 3,4-dithiapentan-1-ol (2.1 g, 16.9 mmol, 26%). The disulphide alcohol had b.p. 155.-160° C./80 Torr. l.r. (liquid film) 3250 cm⁻¹. ¹H n.m.r. (270 MHz) δ2.43, s, 3H; 2.87, t, 2H; 3.08, br s, 1H; 3.88, t. 2H. ¹³C n.m.r. δ23.19, 40.28, 60.26. m/z 124 (63%, M⁺′), 80 (100), 45 (99).

Preparation of 3,4-Dithiapentyl Acetate (14)

3,4-Dithiapentan-1-ol (1.0 g, 8.6 mmol) was added to acetyl chloride (20 ml), and the reaction mixture refluxed for 0-5 h. The solvent was evaporated and the residue rectified at reduced pressure affording clean disulphide acetate (14) (0.44 g, 2.6 mmol, 30%). 3,4-Dithiapentyl acetate (14) had b.p. 135-140° C./18 Torr (Found: C, 36.4; H, 6.4. C₅H₁₀O₂S₂ requires C, 36.1; H, 6.1%). l.r. (liquid film) 1740 cm⁻¹. ¹ H n.m.r. (270 MHz) δ2.08, s, 3H; 2.43, s, 3H; 2.93, t, 2H; 4.34, t, 2H. m/z 166 (2%, M⁺), 87 (51), 43 (100).

The following information and data were published on the website of CSIRO in February, 2000 in the paper mentioned earlier and entitled A New Synthesis for Antifungal α-Sulfone Disulphides.

Compounds (10) and (11) were prepared as shown in. Test results as shown in Table 3 for compounds 8, 9 and 13 appear to establish that the sulfone and disulphide functionalities need be attached to a common carbon for significant fungicidal capacity.

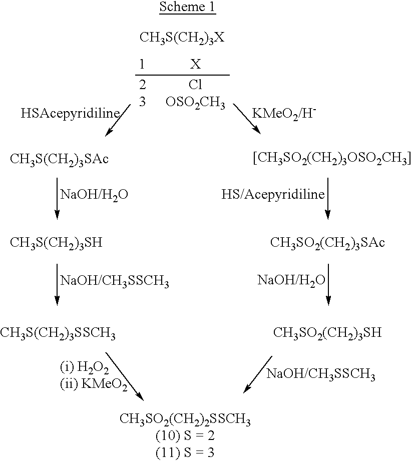

Reaction of the disulphide propionate (2) with the sodium salt of p-toluenesulfinic acid in either aqueous acetonitrile or aqueous acetone leads to smooth displacement of the propionate group (see Scheme 2).

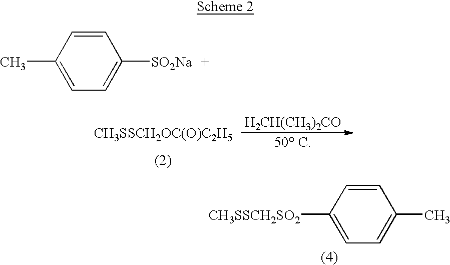

Sequential reaction of (4) with thiophenoxide ions and acetyl chloride produces the thioacetate (6) as depicted in Scheme 3.

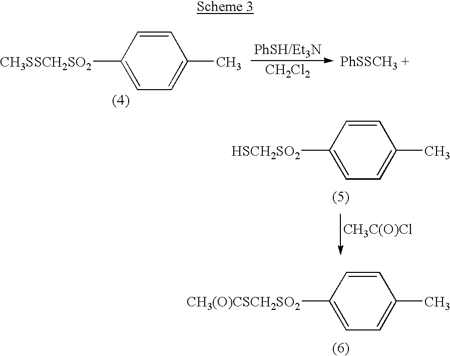

An unambiguous synthesis (see Scheme 4) of the sulfone thioacetate corresponding to (6) proved that (6) is not a thioacetate sulfinate ester.

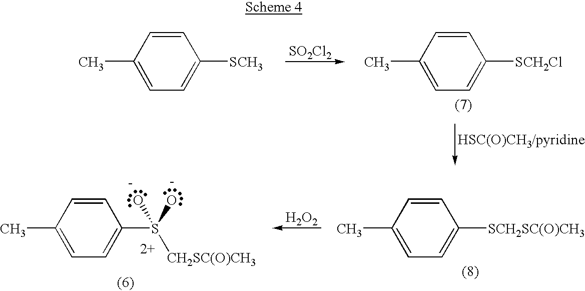

Clearly, sulfinate anion attack on (2) has occurred exclusively with the sulfur atom under our reaction conditions (see Scheme 2).

Schemes 3 and 4 not only establish that (4) is the target α-sulfone disulphide but also establish that (5) is an α-mercapto sulfone.

Typically, preparation of the disulphide ester (2) produces disulphide contaminated with the corresponding sulphide ester (3) (see Scheme 1). Fractional distillation usually leaves a small amount of sulphide ester (3) contaminating distilled disulphide ester (2). It seemed likely that preparation of a sulfone disulphide [e.g. (4), Scheme 2] from (2) would also produce the corresponding sulfone disulphide which would be difficult to remove. However, neither the sulphide propionate (3)³ nor the sulphide acetate (9)3 react with sulfinate anions in warm aqueous acetonitrile (see Scheme 5).

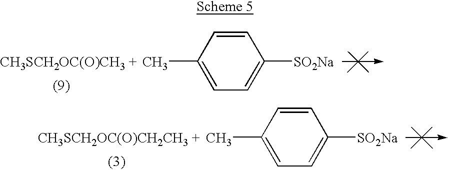

Thus, α-sulfone disulphides (1; R¹═CH₃), prepared as shown in Scheme 2, are readily purified.

Several other α-sulfone disulphides (1) have been prepared from (2) and tested for fungitoxic activity. Antifungal test results for these α-sulfone disulphides and both disulphide propionate (2) and the α-mercaptosulfone (5) are presented in Table 4.

In connection with a related synthetic problem, the disulphide ester (2) was reacted with potassium p-toluenethiosulfonate. This reaction (see Scheme 6) produced the α-sulfone disulphide (4).

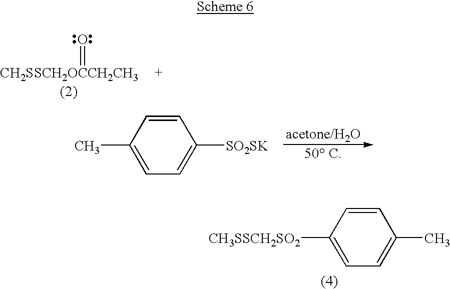

Apparently the reagent is transformed, under the reaction conditions, into the potassium salt of p-toluenesulfinic acid.

Preparation of Compounds of Table 4

Previously Prepared Compounds

Compound (2) was prepared as described in Georges, N. M., Johnson, M. D., Langler, R. F., and Verma, S. D., Sulfur Lett., 1999, 22, 141.

Preparation of α-Sulfone Disulphides (1) (R¹═CH₃)

Table 4 α-sulfone disulphides were prepared in the manner described below for (4).

A solution of sodium p-toluenesulfinate (2.4 g, 13.4 mmol) and the disulphide propionate (2) (2.0 g, 12.0 mmol) in 1:4 water/acetone (30 ml) was immersed in a constant temperature bath at 50° C. for 2 h. Chloroform (150 ml) was added and the resultant mixture washed with water (100 ml). The organic layer was dried (MgSO₄), filtered and the solvent evaporated. The residue was chromatographed on silica gel (200 g) employing 1:1 chloroform/light petroleum (100 ml fractions) for elution. Fractions 13-28 were combined and concentrated furnishing the α-sulfone disulphide (4) (1.6 g, 6.4 mmol, 53%). Recrystallized (methanol) α-sulfone disulphide (4) had m.p. 46.2-48.6° C. (Found: C, 44.1; H, 5.1. C⁹H₁₂O₂S₃ requires C, 43.5; H, 4.9). l.r. 1342, 1148 cm⁻¹. ¹ H n.m.r. (270 MHz) δ2.46, s, 3H; 2.50, s, 3H; 4.20, s, 2H; 7.38, d, 2H; 7.83, d, 2H. ¹³C n.m.r. δ21.70, 23.69, 64.38, 128.98, 129.92, 134.67, 145.36. m/z 248 (6%, M⁺), 139 (56%), 93 (100%).

Oily PhSO₂CH₂SSCH₃ (obtained in 58% yield) has i.r. 1325, 1155 cm⁻¹. ¹H n.m.r. (270 MHz) δ2.49, s, 3H; 4.22, s, 2H; 7.60, t, 2H; 7.70, t, 1H; 7.96, d, 2H. ¹³C n.m.r. δ23.68, 64.32, 128.96, 1 29.30, 134.23, 137.62. m/z 234 (5%, M⁻), 125 (28%), 93 (100%).

Oily CH₃SO₂CH₂SSCH₃^{8,9} (obtained in 53% yield) had i.r. 1320, 1145 cm⁻¹. ¹H n.m.r. (270 MHz) δ2.60, s, 3H; 3.05, s, 2H; 4.16, s, 2H. ¹³C n.m.r. δ23.61, 39.33, 61.38. m/z 172 (10%, M⁺), 93 (100%).

Conversion of (4) into the α-Mercatosulfone (5)

The α-sulfone disulphide (4) (0.20 g, 0.81 mmol) was dissolved in a solution of thiophenol (0.19 g, 1.7 mmol) in dry methylene chloride (10 ml). Dry pyridine (0.1 ml) was added and the reaction mixture stirred at ambient temperature for 2 h 10 min. The solvent was evaporated and the residue chromatographed on silica gel (10 g) employing chloroform (5 ml fractions) for elution. Fractions 3 and 4 were combined and dissolved in chloroform (100 ml). The chloroform layer was washed with 2.5% sodium hydroxide (two 50 ml portions), dried (MgSO₄), filtered and the solvent evaporated. G.l.c./m.s. established the presence of phenyl methyl disulphide and diphenyl disulphide in these fractions. Column fraction 5 furnished a mixture (0.06 g) of unchanged (4) and the α-mercaptosulfone (5). Fractions 6-9 were combined and concentrated yielding clean α-mercaptosulfone (5) (0.09 g, 0.44 mmol, 54%). Recrystallized (5)(methanol) had m.p. 86.4-87.4° C. (Found: C, 47.3; H, 5.0. C₈H₁₀O₂S₂ requires C, 47.5; H, 5.0). l.r. 2500, 1330, 1170 cm⁻¹. ¹ H n.m.r. (270 MHz) δ2.21, t, 1H; 2.46, s, 3H; 3.94, d, 2H; 7.38, d, 2H; 7.83, d, 2H. ¹³C n.m.r. δ21.68 49.43, 129.08, 129.85, 133.67, 145.37.

Conversion of (5) into the Sulfone α-Thioacetate (6)

The α-mercaptosulfone (5) (0.06 g, 0.29 mmol) was covered with acetyl chloride (10 ml) and the reaction mixture refluxed for 1 h. The solvent was evaporated and the residue chromatographed on silica gel (5 g) employing 1:4 light petroleum/methylene chloride (5 ml fractions) for elution. Fractions 6-8 were combined and concentrated giving the sulfone thioacetate (6) (0.024 g, 0.10 mmol, 34%). (Found: C, 49.3%; H, 5.1%. C₁₀H₁₂O₃S₂ requires C, 49.2; H, 5.0). l.r. 1720, 1335, 1165 cm⁻¹. ¹H n.m.r. (270 MHz) δ2.30, s, 3H; 2.45, s, 3H; 4.44, s, 2H; 7.34, d, 2H; 7.81, d, 2H. ¹³C n.m.r. δ21.70, 29.94, 52.09, 128.93, 129.71, 134.20, 145.39, 190.41. m/z 244 (1%, M⁺), 150 (31%), 43 (100%).

Conversion of p-Tolyl Methyl Sulphide into the Sulphide Thioacetate (8)

(A) A solution of p-tolyl methyl sulphide (5.0 g, 36.2 mmol) in dry methylene chloride (50 ml) was refluxed and a solution of sulfuryl chloride (5.0 g, 37.3 mmol) in dry methylene chloride(50 ml) added dropwise over 20 min. The solvent was evaporated and the residue rectified at reduced pressure yielding p-tolyl chloromethyl sulphide (7) (2.6 g, 15.3 mmol, 42%), b.p. 138-142° C./18 Torr. ¹H n.m.r. (270 MHz) δ2.33, s, 3H; 4.88, s, 2H; 7.15, d, 2H; 7.40, d, 2H. ¹³C n.m.r. δ21.11, 51.83, 129.48, 129.97, 131.67, 138.34.

(B) Thioacetic S-acid (0.4 g, 5.8 mmol) was dissolved in dry pyridine (25 ml) and p-tolyl chloromethyl sulphide (1.0 g, 5.8 mmol) added. The reaction mixture was stirred at ambient temperature for 23 h. Chloroform (100 ml) was added and the resultant mixture extracted with 5% HCl (50 ml aliquots) until the aqueous pH remained acidic. The organic layer was extracted with 2.5% NaOH (50 ml), dried (MgSO₄), filtered and the solvent evaporated. The residue was rectified at reduced pressure furnishing the sulphide thioacetate (7) (0.8 g, 3.7 mmol, 64%), b.p. 138-140° C./1.7 Torr. l.r. (liquid film) 1700 cm⁻¹. ¹H n.m.r. (270 MHz) δ2.29, s, 3H; 2.33, s, 3H; 4.29, s, 2H; 7.12, d, 2H; 7.32, d, 2H. ¹³C n.m.r. δ21.11, 30.39, 34.75, 129.82, 130.62, 131.46, 137.69, 194.34. m/z 212 (55%, M⁺), 124 (100%), 91 (55%), 43 (65%).

Conversion of (8) into the Sulfone α-Thioacetate (6)

The sulphide α-thioacetate (8) (1.0 g, 4.7 mmol) and hydrogen peroxide (30%, 1.1 g) were added to 1,4-dioxan (25 ml) and the reaction mixture refluxed for 0.5 h. The solvent was evaporated and chloroform (150 ml) added. The chloroform solution was dried (MgSO₄), filtered and concentrated. The residue was chromatographed on silica gel (100 g) employing chloroform (100 ml fractions) for elution. Fractions 4 and 5 were combined and concentrated affording clean sulfone α-thioacetate (6) (0.34 g, 1.4 mmol, 30%). The product was identical to material described under “Conversion of (5) into the Sulfone α-Thioacetate (6)” by i.r., ¹H n.m.r. (270 MHz) and ¹³C n.m.r.

Reaction of the Disulphide Ester (2) with Potassium p-Toluenethiosulfonate

The disulphide propionate (2) (2.0 g, 12.0 mmol) and potassium p-toluenethiosulfonate (2.7 g, 11.9 mmol) were dissolved in 1:4 water/acetone (30 ml) and the reaction heated at 50° C. for 2 h. Chloroform (200 ml) was added and the resultant mixture extracted with water (100 ml). The organic layer was dried (MgSO₄), filtered and the solvent evaporated. The residue was chromatographed on silica gel (200 g) employing 1:1 chloroform/light petroleum (100 ml fractions) for elution. Fractions 17-22 were combined and concentrated affording the α-sulfone disulphide (4) (0.38 g, 1.5 mmol, 13%) which was identical to (4) described under “Preparation of α-Sulfone Disulphides (1) (R¹═CH₃)” by i.r., ¹H n.m.r. (270 MHz) and ¹³C n.m.r. spectroscopy.

The next targets selected were (3; X=o-SO₂CH₃) and (3; X=p-SO₂CH₃). Given that benzenesulfonyl chlorides can generally be reduced (LiAlH₄) to the corresponding mercaptans (Fong, H. O., Hardstaff, W. R., Kay, D. G., Langler, R. F., Morse, R. G., and Sandoval, D. N., Can. J. Chem., 1979, 57m 1206), we elected to prepare the appropriate methylsulfonyl-substituted benzenesulfonyl chlorides (14) and (15) (Ginige, K. A., Goehl, J. E., and Langler, R. F., Can. J. Chem., 1996, 74, 1638). Lithium aluminum hydride reduction, followed by attempted thiomethylation, as shown in Scheme 7, gave none of the target methyl disulphides, but instead furnished the symmetrical disulphides (7) and (8).

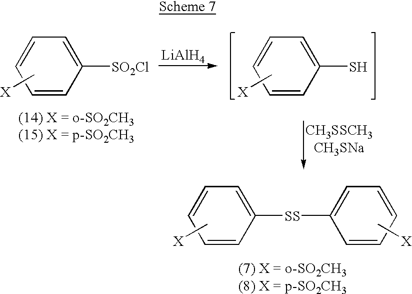

The disulfone disulphides (7) and (8) are fungitoxic (see Table 5).

The next set of target disulphides included (3; X=o-NO₂), (3; X=m-NO₂) and (3; X=p-NO2). Each of these molecules contains a most powerful electron withdrawing group and so, were expected to be potent antifungal disulphides. The results for those compounds ((9), (10) and (11) in Table 1) are in complete accord with expectations.

Appropriately substituted nitrophenyl or methylsulfonylphenyl aromatics can undergo smooth nucleophilic aromatic substitutions which arylate thiolate anions (Ginige, K. A., Goehl, J. E., and Langler, R. F., Can. J. Chem., 1996, 74, 1638, Baum, J. C., Bolhassan, J., Langler, R. F., Pujol, R. J., and Raheja, R. K., Can. J. Chem., 1990, 68, 1450) in dimethyl sulfoxide or hexamethylphosphoramide (e.g. see Scheme 8).

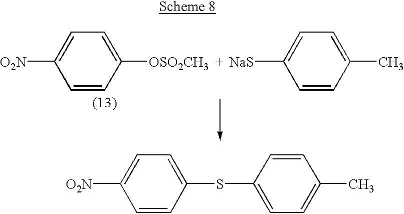

Preparation of Compounds of Table 5

The test results (Table 5) for compounds (12) and (13) demonstrate that potential nucleophilic aromatic substitutions which arylate by displacement of the entire disulphide linkage do not inhibit fungal growth.

Previously Prepared Compounds

Compound (12) was prepared as described in Ginige, K. A., Goehl, J. E., and Langler, R. F., Can. J. Chem., 1996, 74, 1638 and compound (13) was prepared as outlined in Baum, J. C., Bolhassan, J., Langler, R. F., Pujol, R. J., and Raheja, R. K., Can. J. Chem., 1990, 68, 1450.

Preparation of Phenyl Methyl Disulphide

Sodium metal (0.02 g, 0.69 mmol) was dissolved in methanol (1 ml) and thiophenol (0.1 ml) added. The solvent was evaporated and the sodium thiophenate dried in vacuo. The thiophenate salt was dissolved in dimethyl sulfoxide (10 ml). A portion of the resultant solution (1 ml) was added to a mixture of diphenyl disulphide (1.98 g, 9.1 mmol) and dimethyl disulphide (12 ml). The reaction mixture was stirred at ambient temperature for 8 days.

2.5% Hydrochloric acid (70 ml) was added and the resultant mixture washed with diethyl ether (three—50 ml aliquots). The organic layers were combined, dried (MgSO₄), filtered and the solvent evaporated. The concentrate was rectified at reduced pressure affording phenyl methyl disulphide (2.38 g, 15.2 mmol, 84%), b.p. 82-86° C./1.8 Torr. ¹H n.m.r. (270 MHz) δ7.51, d, 2H; 7.30, t, 2H; 7.20, t, 1H; 2.40, s, 3H. ¹³C n.m.r. δ22.82 126.77, 127.47, 128.96, 136.83. m/z 156 (100%, M+), 141 (65%) and 109 (57%).

Preparation of Methyl o-Mercaptobenzoate

o-Mercaptobenzoic acid (9.9 g, 64.2 mmol) was dissolved in methanol (300 ml) and concentrated sulfuric acid (0.5 ml) added. The reaction mixture was refluxed for 72 h. Chloroform (300 ml) was added and the resultant mixture extracted with water (two—100 ml aliquots) and 1% sodium hydroxide (two—100 ml aliquots). The combined aqueous layers were added to chloroform (75 ml), ice (100 ml) and concentrated hydrochloric acid (8 ml). Chloroform (150 ml) was added, the layers separated, the organic layer dried (MgSO₄) and filtered. The solvent was evaporated and the residue rectified at reduced pressure affording clean o-mercapto methyl benzoate (6.9 g, 41.0 mmol, 64%), b.p. 103-108° C./2.7 Torr. l.r. (liquid film) 1710 cm⁻¹. ¹H n.m.r. (270 MHz) δ7.99, d, 1H; 7.29, d, 1H; 7.14, m, 2H; 4.68, s, 1H; 3.90, s, 3H. ¹³C n.m.r. δ167.09, 138.27, 132.45, 131.65, 130.86, 125.74, 124.62, 52.19. m/z 168 (21%, M⁺), 136 (100%), 108 (35%).

Preparation of o-Carbomethoxyphenyl Methyl Disulphide (6)

Powdered sodium hydroxide (0.24 g, 6 mmol) was suspended in dimethyl sulfoxide (8 ml) and a solution of o-mercapto methyl benzoate (1.0 g, 5.9 mmol) in dimethyl sulfoxide (5 ml) added. The reaction mixture was stirred for 5 min and a solution of dimethyl disulphide (1.7 g, 18 mmol) in dimethyl sulfoxide (7 ml) added. The reaction mixture was stirred for 24 h at ambient temperature.

2.5% Hydrochloric acid (150 ml) was added to the reaction and the resultant mixture extracted with diethyl ether (three—100 ml aliquots). The organic layers were combined and concentrated. 2.5% Hydrochloric acid (150 ml) was added to the concentrate and the resultant mixture washed with diethyl ether (three—100 ml aliquots). The organic layers were combined and concentrated. 2.5% (WV) Sodium hydroxide solution (150 ml) was added to the residue and the resultant mixture extracted with diethyl ether (three—100 ml aliquots). The combined organic layers were dried (MgSO₄), filtered and the solvent evaporated. The crude product was chromatographed on silica gel (100 g) employing 3:2 chloroform/light petroleum (100 ml fractions). Fractions 3 and 4 were combined and concentrated and the product rectified at reduced pressure giving clean (6) (0.0 g, 0.4 mmol, 7%), b.p. 141-142° C./2.1 Torr (Found: C, 50.5; H, 4.6. C₉H₁₀O₂S₂ requires C, 50.4; H, 4.7). l.r. (liquid film) 1705 cm⁻¹. ¹H n.m.r. (270 MHz) δ8.15, d, 1H; 8.04, d, 1H; 7.58, t, 1H; 7.25, t, 1H; 3.93, s, 3H; 2.40, s, 3H. ¹³C n.m.r. δ166.80, 141.30, 132.92, 131.57, 126.90, 125.09, 52.29, 21.99. m/z 214 (35%, M⁺), 167 (100%), 152 (37%), 136 (41%).

Preparation of o-Chlorophenyl Methyl Sulphide

Sodium metal (0.80 g, 34 mmol) was dissolved in methanol (80 ml) and a solution of o-chlorothiophenol (5.1 g, 35 mmol) in methanol (10 ml) added. The reaction mixture was cooled with an ice/water bath and a solution of methyl iodide (5.0 g, 35 mmol) in methanol (10 ml) was added dropwise. The reaction mixture was stirred at ambient temperature for 24 h. Water (100 ml) was added and the resultant mixture extracted with chloroform (three—100 ml aliquots). The organic layers were combined, dried (MgSO₄) and the solvent evaporated. The residue was distilled at reduced pressure yielding o-chlorophenyl methyl sulphide (4.7 g, 29.7 mmol, 85%), b.p. 80-86° C./3.0 Torr. ¹H n.m.r. (270 MHz) δ7.33, d, 1H; 7.22, t, 1H; 7.12, d, 1H; 7.07, t, 1H; 2.47, s, 3H. ¹³C n.m.r. δ137.70, 131.75 129.69, 129.34, 127.18, 125.46, 15.13. m/z 160 (33%),158 (100%, M⁺), 145 (24%), 143 (66%).

Preparation of o-Chlorophenyl Methyl Sulfone

o-Chlorophenyl methyl sulphide (4.1 g, 25.9 mmol) in chloroform (86 ml) was added dropwise to 10% sulfuric acid (120 ml). Simultaneously, potassium permanganate (13.9 g) was added in small portions. The double addition took 45 min. Upon completion of the addition, the reaction mixture was stirred at ambient temperature for 1 h. The reaction mixture was cooled in an ice/water bath and sodium bisulfite added until the reaction mixture became colorless. The layers were separated and the aqueous layer extracted with chloroform (three—100 ml portions). The combined organic layers were dried (MgSO₄), filtered and the solvent evaporated. Crude chlorosulfone was recrystallized (methanol) affording clean chlorosulfone (4.0 g, 21.0 mmol, 81%), m.p. 93.5-95.1° C. l.r.1330, 1165cm⁻¹. ¹H n.m.r. (270 MHz) δ8.15, d, 1H; 7.58, m, 2H; 7.48, m, 1H; 3.29, s, 3H. ¹³C n.m.r. δ138.03, 134.79, 131.91, 130.84, 127.52, 42.73. m/z 192 (7%), 190 (20%, M⁺), 113 (33%), 111 (100%).

Preparation of o-Methylsulfonylphenyl Benzyl Sulphide

Sodium metal (0.24 g, 10.4 mmol) was dissolved in methanol (5 ml) and benzyl thiol (1.3 ml) added. The solvent was evaporated and sodium benzyl thiolate dried in vacuo. The sodium benzyl thiolate was dissolved in dimethyl sulfoxide (50 ml) and o-chlorophenyl methyl sulfone (2.0 g, 10.6 mmol) added. The reaction mixture was stirred at ambient temperature for 19 h. 10% Hydrochloric acid (200 ml) was added and the product filtered off. Dried sulfone sulphide (2.0 g) was recrystallized (methanol) furnishing o-methylsulfonylphenyl benzyl sulphide (1.6 g, 5.8 mmol, 55%), m.p. 132.0-133.4° C. l.r. 1315, 1155 cm⁻¹. ¹H n.m.r. (270 MHz) δ8.07, d, 1H; 7.50, m, 2H; 7.37, m, 6H; 4.24, s, 2H; 3.17, s, 3H. ¹³C n.m.r. δ139.47, 137.15, 136.08, 133.59, 131.05, 130.10, 128.93, 128.70, 127.67, 126.42, 42.05, 39.28. m/z 278 (3%, M⁺), 91 (100%).

Preparation of o-Chlorosulfonylphenyl Methyl Sulfone (14)

o-Methylsulfonylphenyl benzyl sulphide (1.0 g, 3.5 mmol) was suspended in glacial acetic acid (35 ml) and water (3ml). Cl₂ (ca 200 ml/min) was bubbled into the reaction mixture for 45 min. Ice/water cooling was employed, as necessary, to maintain the reaction temperature below 30° C. Chloroform (100 ml) was added and the resultant mixture extracted with 2.5% (WV) sodium hydroxide (three -50 ml aliquots). The organic layer was dried (MgSO₄), filtered and the solvent evaporated. The sulfone sulfonyl chloride was recrystallized (dry carbon tetrachloride) yielding o-chlorosulfonylphenyl methyl sulfone (0.64 g, 2.5 mmol, 71%), m.p. 140.4-142.2° C. l.r. 1380, 1330, 1155 cm⁻¹. ¹H n.m.r. (270 MHz) δ8.42, t, 2H; 7.95, m, 2H; 3.41, s, 3H. ¹³C n.m.r. δ142.80, 139.08, 135.99, 134.64, 133.18, 131.72, 45.12.

Preparation of the Disulfone Disulphides (7) and (8)

Both sulfone disulphides were prepared as described below for di-o-methylsulfonylphenyl disulphide (7). Note that the preparation of p-chlorosulfonylphenyl methyl sulfone has been described earlier.

(A) Lithium aluminum hydride (1.2 g, 31.5 mmol) was added to tetrahydrofuran (20 ml). A solution of o-chlorosulfonylphenyl methyl sulfone (2.0 g, 7.8 mmol) in tetrahydrofuran (80 ml) was added dropwise over 25 min. The reaction mixture was refluxed for 1 h. After cooling to ambient temperature the following chemicals were added sequentially in a dropwise manner: ethyl acetate (20 ml), methanol (10 ml), water (10 ml), 1% hydrochloric acid (40 ml) and concentrated hydrochloric acid(12 ml). Chloroform (250 ml) was added and the resultant mixture washed with water (two—150 ml aliquots). The organic layer was dried (MgSO₄), filtered and concentrated affording crude phenyl methyl sulfone (0.33 g). Phenyl methyl sulfone was recrystallized (methanol) and shown to be identical to authentic material by m.p., mixture m.p., i.r. and ¹H n.m.r. (60 MHz).

The aqueous layer from the extraction procedure was acidified (12 ml of concentrated hydrochloric acid) and the resultant mixture extracted with chloroform (three—100 ml aliquots). The combined organic layers were dried (MgSO₄), filtered and the solvent evaporated affording crude oily o-mercaptophenyl methyl sulfone (0.6 g).

(B) Sodium metal (0.25 g, 10.7 mmol) was dissolved in methanol (25 ml) and methanethiol (250 ml) bubbled into the solution. The solvent was evaporated and the sodium methanethiolate dried in vacuo. The sodium methanethiolate was dissolved in dimethyl sulfoxide (15 ml) and a solution of oily o-mercaptophenyl methyl sulfone (1.9 g, 10 mmol), dimethyl disulphide (3.0 g, 31 mmol) and dimethyl sulfoxide (5 ml) added. The reaction mixture was stirred at ambient temperature for 20 h. 2.5% Hydrochloric acid (150 ml) was added and the resultant mixture extracted with diethyl ether (three—100 ml aliquots). The organic layers were combined, dried (MgSO₄), filtered and the solvent evaporated. Crude disulfone disulphide (7) was recrystallized from methanol (175 ml).

Clean disulfone disulphide (7) (0.69 g, 1.8 mmol, 46% from the sulfonyl chloride) had m.p. 225-227° C. (Found: C, 45.0; H, 3.8. Cl₄H₁₄O₄S₄ requires C, 44.9; H, 3.8) l.r. (KBr) 1300, 1140 cm⁻¹. ¹H n.m.r. (DMSO-d₆, 270 MHz) δ8.06, d, 1H; 7.86, m, 2H; 7.65, t, 1H; 3.44, s, 3H. ¹³C n.m.r. (DMSO-d₆) δ138.22, 135.67, 134.92, 130.04, 127.90, 127.64, 42.52. m/z 374 (21%, M⁻), 296 (11%), 234 (15%), 188 (100%).

Clean disulfone disulphide (8) (22% from the sulfonyl chloride) had m.p. 183-185° C. (Found: C, 45.2; H, 3.9. C₁₄H₁₄O₄S₄ requires C, 44.9; H. 3.8). l.r. (KBr) 1308, 1155 cm⁻¹. ¹H; n.m.r. (DMSO-d₆, 270 MHz) δ7.93, d, 4H; 7.80, d, 4H; 3.22, s, 6H. ¹³C n.m.r. (DMSO-d₆) δ141.61, 139.52, 128.05, 126.45, 43.33. m/z 374 (31%, M⁺), 234 (18%), 188 (100%).

Preparation of the Nitrophenyl Disulphides (9), (10) and (11)

The nitrophenyl methyl disulphides were prepared from the appropriate symmetrical di(nitrophenyl) disulphides as described below for the para-nitro case.

Sodium metal (0.018 g, 0.78 mmol) was dissolved in methanol (10 ml) and methanethiol (20 ml) bubbled into the solution. The solvent was evaporated and the sodium methanethiolate dried in vacuo.

The sodium methanethiolate was dissolved in dimethyl sulfoxide (10 ml). Di(p-nitrophenyl) disulphide (2.0 g, 6.6 mmol) was added to dimethyl sulfoxide (2 ml) and a portion of the methanethiolate solution (1 ml) added. Dimethyl disulphide (12 ml) was added and the reaction mixture stirred at ambient temperature for 8 days. The reaction mixture became homogeneous after stirring for 24 h.

2.5% Hydrochloric acid (70 ml) was added and the resultant mixture extracted with diethyl ether (three—50 ml aliquots). The organic layers were combined, dried (MgSO₄), filtered and rotary evaporated. The residue was chromatographed on silica gel (50 g) employing light petroleum (twelve—50 ml fractions) followed by 1:1 light petroleum/chloroform (50 ml fractions) for elution. Fractions 13-22 were combined and rectified at reduced pressure affording p-nitrophenyl methyl disulphide (11) (1.2 g, 5.9 mmol, 45%), b.p. 146-149° C./0.9 Torr. (11) crystallized on standing and after recrystallization (methanol) had m.p. 42.9-44.3° C.

Clean o-nitrophenyl methyl disulphide (10) (31%) had m.p. 49-51° C. (Found: C, 41.9; H, 3.3. C₇H₇NO₂S₂ requires C, 41.8; H, 3.5). l.r. 1524, 1340 cm⁻¹. ¹H n.m.r. (270 MHz) δ8.29, t, 2H; 7.71, t, 1H; 7.37, t, 1H; 2.43, s, 3H. ¹³C n.m.r. δ137.25, 134.10, 126.79, 126.09, 21.89. m/z 201 (14%, M⁺), 136 (100%), 122 (41%).

Clean m-nitrophenyl methyl disulphide (9) (74%) had b.p. 152-158° C./2 Torr (Found: C, 42.0; H, 3.6. C₇H₇NO₂S₂ requires C, 41.8; H, 3.5). l.r. 1530, 1350 cm⁻¹. ¹H (270 MHz) δ8.39, s,1H; 8.04, d, 1H; 7.80, d, 1H; 7.52, t, 1H; 2.49, s, 3H. ¹³C n.m.r. δ148.78, 140.10, 132.26, 129.79, 121.37, 121.07, 22.85. m/z 201 (100%, M⁺), 140 (48%).

Clean p-nitrophenyl methyl disulphide (11) (45%) had C, 41.9; H, 3.5. C₇H₇NO₂S₂ requires C, 41.8; H, 3.5. l.r. 1515, 1340 cm¹. ¹H n.m.r. (270 MHz) δ8.20, d, 2H; 7.66, d, 2H; 2.48, s, 3H. ¹³C n.m.r. δ146.43, 125.73, 124.15, 22.71. m/z 201 (100%, M⁺), 140 (56%).

Preparation of Compounds of Table 6

An older example of α-ester disulphide preparation which applied a Pummerer reaction to a thiosulfinate (Saito, I., and Fukui, S., J. Vitaminol. (Kyoto), 1966, 12, 244) is illustrated by the following reaction scheme (see Scheme 9).

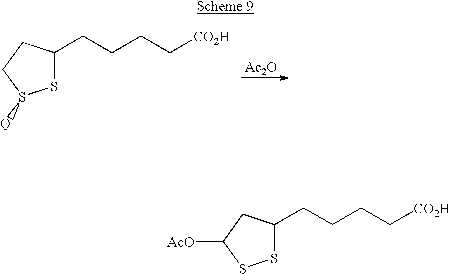

The foregoing reaction appears to proceed through the intermediacy of an acetoxy sulfonium ion. Therefore the direct reaction of a disulphide with dibenzoyl peroxide should produce a benzoyloxy sulfonium ion and, from there, an α-ester disulphide. Relative to prior disclosed reaction schemes, dibenzoyl peroxide provided a significant improvement in the yield of an α-ester disulphide as shown in Scheme 10.

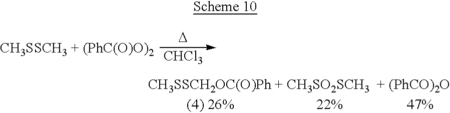

Although antifungal testing on (4) showed it to be very potent (see Table 6), it was less fungitoxic than several other disulphides, including (3), that have been described earlier (Baerlocher, F. J., Langler, R. F., Frederiksen, M. U., Georges, N. M., and Witherell, R. D., Aust. J. Chem., 1999, 52,167; Langler, R. F., MacQuarrie, S. L., McNamara, R. A., and O'Connor, P. E., Aust J. Chem. 52, 1119 (1999); and Baerlocher, F. J., Baerlocher, M. O., Langler, R. F., MacQuarrie, S. L., and Marchand, M. E., Aust J. Chem. 53, 1200).

In order to examine a structural variant of (4), the α-ester disulphide PhSSCH₂OC(O)CH₂CH₃ (5) was synthesized. Unfortunately, potassium permanganate/propionic acid oxidation of phenyl methyl disulphide only furnishes (5) in 0.9% yield. Consequently, an alternative two-step synthesis from (3) was developed (vide Scheme 11).

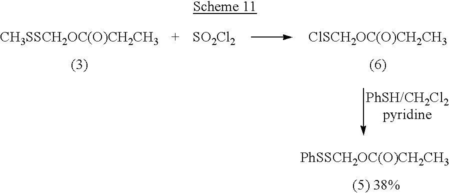

Compound (2) and (3) are known compounds. Methods of preparing these compounds are also known, these methods having been described in Georges, N. M Johnson, M. D., Langler, R. F., and Verma, S. D., Sulfur Lett. 22, 141 (1999).

Preparation of Disulphide Benzoate (4)

Five parallel reactions were conducted as follows. A solution of dimethyl disulphide (1.3 g, 13.8 mmol) and dibenzoyl peroxide (5.1 g, 21.0 mmol) in chloroform (60 ml) was refluxed behind a safety shield for 24 h.

Upon completion of the reflux period, the five runs were combined and chloroform (250 ml) was added. The resultant solution was extracted with 2.5% sodium hydroxide solution (two—125 ml aliquots). The organic layer was dried (MgSO₄), filtered and the solvent evaporated. The crude product was rectified at reduced pressure affording a low-boiling fraction (2.0 g, b.p. 25-80° C./1.9 Torr). A portion of the low-boiling fraction (1.0 g) was chromatographed on silica gel employing 1:1 chloroform/light petroleum (100 ml fractions) for elution. Methyl methanethiosulfonate (0.08 g) was obtained.

Based on g.l.c./m.s., the high-boiling distillation fraction (b.p. 80-140° C./1.6 Torr) contained a mixture of the disulphide benzoate (4) (2.1 g, 9.8 mmol) and dibenzoyl anhydride (2.1 g). In the same manner, the distillation residue was determined to contain the disulphide benzoate (4) (1.5 g, 7.0 mmol) and dibenzoyl anhydride (9.1 g). Distillation, at atmospheric pressure, of the condensate from the cold trap, furnished a mixture which contained methyl methanethiosulfonate (0.7 g).

A sample of the higher-boiling distillation fraction (2 g) was chromatographed on silica gel (200 g) employing 3:1 light petroleum/chloroform (100 ml fractions) for elution. Fractions 9-12 were combined and concentrated affording disulphide benzoate (4). The disulphide (4)(0.6 g) had b.p. 139° C./2 Torr (Found: C, 50.7; H, 4.6. C₉H₁₀O₂S₂ requires C, 50.4; H, 4.7). l.r. 1720 cm⁻¹. ¹H n.m.r. (270 MHz) δ2.51, s, 3H; 5.55, s, 2H; 7.47, t, 2H; 7.60, m, 1H; 8.07, d, 2H. ¹³C n.m.r. δ24.48, 73.55, 128.52, 129.51, 129.77, 133.4, 165.76. m/z 184 (9%, M⁺—CH₂O),105 (100), 77 (36).

Preparation of the Sulfenyl Chloride (6)

A solution of sulfuryl chloride (1.6 g, 11.9 mmol) in dry methylene chloride (10 ml) was added to a solution of the disulphide propionate (3)² (2.0 g, 12 mmol) in dry methylene chloride (6 ml). The reaction mixture was refluxed for 0.5 h and the solvent carefully evaporated.

Crude product was distilled at reduced pressure affording impure sulfenyl chloride (6) (0.8 g). The sulfenyl chloride (6) had b.p. 94-114° C./53 Torr and was not further purified. Impure sulfenyl chloride (6) had i.r. 1750 cm⁻¹. ¹H n.m.r. (270 MHz) showed major signals at δ1.18, t, 3H; 2.42, q, 2H; 5.60, s, 2H. ¹³C n.m.r. showed major signals at δ8.84, 27.39, 72.31, 173.76. m/z 154 (6%, M⁺) 118 (11), 57 (100). The sulfenyl chloride (6) was routinely stored in the freezer.

Preparation of the Propionate Disulphide (5)

Distilled impure sulfenyl chloride (6) (1.0 g) was added to a solution of benzene thiol (0.7 g, 6.3 mmol) and pyridine (1 ml) in dry methylene chloride (15 ml). The reaction mixture was stirred at ambient temperature for 2 h. Chloroform (100 ml) was added and the resultant mixture washed with 2.5% HCl (100 ml) and then 2.5% sodium hydroxide solution (100 ml). The organic layer was dried (MgSO₄), filtered and the solvent evaporated. The residue was chromatographed on silica gel (100 g) employing light petroleum (twenty—100 ml fractions) followed by chloroform (100 ml fractions). Fraction 22 furnished clean disulphide propionate (5) (0.53 g. 2.3 mmol).

The reaction was repeated on impure sulfenyl chloride (6) (2.0 g) and the chromatographed (5) so-obtained was added to the chromatographed product from the first run. The disulphide was rectified at reduced pressure affording clean (5) (1.7 g, 7.4 mmol) whose properties were in full accord with those expected.

Preparation of Compounds of Table 7

Preparation of (CH₃OC(O)CH₂SSCH₂C(O)C₂H₅) methyl 3,4-dithia-5-propionoxypentanoate

Methyl thioglycollate (1.896 g, 15.8 mmol) was added to dry methylene chloride (10 ml). ClSCH₂OC(O)C₂H₅ (2.44 g, 15.6 mmol) was dissolved in dry methylene chloride and the resultant solution added to the reaction mixture. Dry pyridine (2.5 ml) was added and the reaction mixture stirred at ambient temperature for 24 h. Chloroform (200 ml) was added to the reaction and the resultant solution washed first with 2.5% hydrochloric acid (200 ml) then. with 2.5% sodium hydroxide (200 ml). The organic layer was dried (MgSO₄), filtered and concentrated.

The residue was chromatographed on silica gel (250 g) employing petroleum ether (200 ml fractions) for fractions 1-19, then chloroform (200 ml fractions). Fraction 23 was concentrated and rectified at reduced pressure affording CH₃OC(O)CH₂SSCH₂OC(O)C₂H₅ (0.53 g, bp 140-150° C./4.5 Torr). l.r. 1755 cm⁻¹. ¹H n.m.r. (270 MHz) δ1.17, t, 3H; 2.40, g, 2H; 3.58, s, 2H; 3.77, s, 3H; 5.32, s, 2H. ¹³C n.m.r. δ8.84, 27.55, 41.76, 52.63, 72.65, 169.54, 173.63. m/z 224 (1%, M^{t},), 194 (41%), 57 (100%).

Preparation of ((C₂H₅OC(O)CH₂S)₂)2,3-dithiabutane-1,4-dipropionate

(CH₃SSCH₂OC(O)C₂H₅) 2,3-dithiabutylpropionate (18.029 g, 109 mmol) was added to propionic acid (350 ml) and the solution refluxed. Portions of potassium permanganate (ca 3 g each, 18.081 g in total) were added. The solution would immediately turn brown upon the addition of an aliquot of permanganate but would turn white thereafter. Provided the color change took place in less than 5 min, another 3 g portion of permanganate was added. When the color change took longer than 5 min, smaller portions (ca 0.5 g each) were added at 5 min intervals. Propionic acid (ca 5 ml) was used to rinse each aliquot of permanganate into the reaction mixture. When the last addition had been done (elapsed time 1 h), the reaction mixture was cooled in an ice/water bath.

Chloroform (500 ml) was added and the resultant mixture washed with 10% sodium hydroxide solution (six—250 ml portions), after which the aqueous pH remained basic. The organic layer was dried (MgSO₄), filtered and concentrated. The residue was rectified at reduced pressure affording unchanged starting material (4.342 g, bp 161-189° C./1 Torr) and (C₂H₅C(O)OCH₂S)₂ (5.061 g, bp 110-122° C./0.7 Torr).

(C₂H₅C(O)OCH₂S)₂ had l.r. 1750 cm¹. ¹H n.m.r. (270 MHz) δ1.18, t, 3H; 2.42, q, 2H; 5.28, s, 2H. ¹³C n.m.r. δ27.5, 72.7, 173.5. m/z 208 (14%), 57 (100%).

Preparation of (C₂H₅C(O)OCH₂SSCH₂SO₂(C₆H₄) CH—) 1-p-toluenesulfonyl-4-propionoxy-2,3-dithiabutane

((C₂H₅C(O)OCH₂S)₂) 2,3-dithiabutane-1,4-dipropionate (0.507 g) was dissolved in a solution of acetone/water (4:1 respectively, 30 ml). Sodium p-toluenesulfinate polyhydrate (0.411 g) was added and the reaction mixture heated at 49° C. for 2 h. At the end of this period, the reaction mixture was dark orange in color.

Water (100 ml) was added and the resultant mixture washed with chloroform (three 50 ml portions). The combined organic layers were dried (MgSO₄) and concentrated. Crude product was chromatographed on silica gel (50 g), employing chloroform for elution. Fractions 10-12 were combined and concentrated affording the product (0.21 g).

C₂H₅C(O)OCH₂SSCH₂SO₂(C₆H₄)CH₃-p had l.r. 1749, 1326, 1151 cm⁻¹. ¹H n.m.r. δ1.16, t, 3H; 2.38, q, 2H; 2.47, s, 3H; 4.24, s, 2H; 5.34, s, ZH; 7.38, d, 2H; 7.80, d, 2H. ¹³C n.m.r. δ8.82, 21.70, 27.41, 63.93, 72.38, 128.99, 129.94, 134.43, 145.49, 173.43. m/z 290 (13.7%), 57 (100%).

Preparation of Compound Q, 1-phenyl-1,2-dithiapropyl propionate, PhSSCH₂OC(O)C₂H₅ (See Table 6)

Compound Q is a potent antifungal compound. It's preparation is described in the preprint “New Antifungal Disulphides: Approaching Submicrogram Toxicity”, F. J. Baerlocher,, M. O. Baerlocher, C. L. Chaulk, R. F. Langler and E. M. O'Brien, Sulfur Lett.,—in press, the disclosures of which are incorporated herein by reference. See earlier description relative to Scheme 5 for method.

Preparation of N, 2,3,5-trithiahexane, CH₃SCH₂SSCH₃ (See Table 3)

Sodium metal (0.020 g) was dissolved in methanol (2 ml) and methanethiol (20 ml) slowly bubbled into the solution. The resultant solution was concentrated and the residue dried in vacuuo. The resulting solid was dissolved in DMSO (10 ml). A portion of this solution (1 ml) was added to a mixture of dimethyl disulphide (12 ml) and (CH₃SCH₂S)₂ (2.00 g)(preparation—P. Dubs and R. Stuessi, Helv. Chim. Acta 61, 2351 (1978)). The reaction mixture was stirred at ambient temperature for eight days.

2.5% Hydrochloric acid (70 ml) was added and the resultant mixture extracted with diethyl ether (three—50 ml aliquots). The organic layers were combined and dried (MgSO₄), filtered and concentrated. The concentrate was distilled at reduced pressure yielding N (2.336 g, bp. 92-102° C./18 Torr). N had ¹H n.m.r. (270 MHz) δ2.22, s, 3H; 2.49, s, 3H; 3.86, s, 2H. ¹³C n.m.r. δ65.12, 73.39, 94.23.

Preparation of P, phenacyl methyl disulphide, PhC(O)CH₂SSCH₃

A) Phenacyl chloride (1.004 g) was added to dry pyridine (4 ml). Thiolacetic acid (0.560 g) was dissulved in dry pyridine (6 ml) and added to the reaction mixture. The reaction flask was fitted with a drying tube and the reaction mixture heated at 800° C. for 1.5 h. Chloroform (200 ml) was added and the resultant mixture extracted with 5% hydrochloric acid (150 ml), followed by 2.5% sodium hydroxide (100 ml). The organic layer was dried (MgSO₄), filtered and concentrated. The product was chromatographed on silica gel (5 g) employing petroleum ether (400 ml) for elution. Evaporation of the solvent afforded an orange oil.

Fractional distillation provided phenacyl thiolacetate (0.959 g, bp. 137-139° C./2.4 Torr). l.r. 1710, 1685 cm⁻¹. ¹H n.m.r. (270 MHz) δ2.38, s, 3H; 4.39, s, 2H; 7.46, t, ZH; 7.58, t, 1H; 7.97, d., 2H. ¹³C n.m.r. δ30.17, 36.62, 128.44, 128.74, 133.67, 135.50, 193.10, 194.08. m/z 194 (M^{t}., 3%), 105 (100%).

B) Potassium carbonate (19.00 g) was added to methanol (147 ml) which was cooled (O° C.) and stirred 1 h. Phenacyl thiolacetate (4.157 g) was added to the reaction vessel dropwise. Cooling and stirring continued for another 30 min. Diethyl ether (205 ml) and water (200 ml) were added and the reaction mixture cooled for another 30 min. Iodine (3.590 g) was added in small portions over 30 min. Saturated sodium thiosulfate solution (16 ml) was added.

Diethyl ether (85 ml) was added to the reaction mixture and the layers separated. The organic layer was washed with distilled water (two—200 ml aliquots. The organic layer was dried (MgSO₄), filtered and concentrated in vacuuo. The product was chromatographed on silica gel (400 g) employing chloroform for elution (100 ml fractions). Fractions 16-29 were combined and concentrated. The concentrate was recrystallized from methanol/benzene which produced a crop of gummy crystals (0.551 g). A second recrystallization furnished phenacyl disulphide with the following properties. l.r. 1675 cm⁻¹. ¹H n.m.r. 64.20, s, 4H; 7.46, t, 4H; 7.59, t, 2H; 7.94, d, 4H. ¹³C n.m.r. δ45.36, 128.74, 128.77, 133.68, 135.35, 194.30. m/z 105 (100%).

C) Sodium metal (0.019 g) was dissolved in methanol (10 ml). Methanethiol (20 ml) was bubbled through the solution. The solvent was evaporated and the residue dried in vaccuo. DMSO (10 ml) was added to the solid and the mixture stirred for 30 min.

Phenacyl disulphide (1.603 g) and dimethyl disulphide were combined and a portion of the DMSO solution (1 ml) was added. The reaction mixture was stirred at ambient temperature for 8 days. 2.5% Hydrochloric acid (70 ml) was added. The resultant mixture was extracted with diethyl ether (three—50 ml aliquots). The combined organic layers were dried (MgSO₄), filtered and concentrated.

The crude product was chromatographed on silica gel (200 g) employing 3:7 chloroform/petroleum ether (100 ml fractions) for elution. Fractions 12-19 were combined and concentrated. The residue was rectified at reduced pressure furnishing phenacyl methyl disulphide (1.121 g, bp. 144-154° C./1-7 Torr). It had l.r. 1690 cm⁻¹. ¹H n.m.r. δ2.37, s, 3H; 4.09, s, 2H; 7.47, t, 2H; 7.59, t, 1H); 7.97, d, 2H. ¹³C n.m.r. δ22.93, 44.25, 128.7, 128.73, 133.51, 135.23, 194.44. m/z 198 (M^{t}, 15%), 105 (100%).

Preparation of Compounds in Table 8

Previously Prepared Compounds

Compound (2) was prepared as described earlier (Langler, R. F., MacQuarrie, S. L., McNamara, R. A., and O'C.onnor, P. E., Aust. J. Chem.—in press). The preparations of compounds (3), (12), (17) and (7) were outlined previously (Baerlocher, F. J., Baerlocher, M. O., Langler, R. F., MacQuarrie, S. L., and Marchand, M. E., Aust J. Chem.—in press). The synthesis of compound (20) has been reported (Baerlocher, F. J., Langler, R. F., Frederiksen, M. U., Georges, N. M., and Witherell, R. D., Aust. J. Chem., 1999, 52,167. Syntheses for compounds (4), (13), (15) and (16) have been described in refs.: Oae, S., Takata, T., and Kim, Y. H., Bull. C. S. Jpn., 1982, 55,2484; Goodridge, R. J., Hambley, T. W., and Haynes, R. K., J. Org. Chem.,1988, 53, 2881; Langler, R. F., Ryan, D. A., and Verma, S. D., Sulfur Lett. 24, 51 (2000); and Back, T. G., Collins, S., and Krishna, M. V., Can. J. Chem., 1987, 65, 38, respectively.

Three Approaches to the Synthesis of Phenyl Methanethiosulfonate (4)

(A) Disulphide Oxidation

Methyl phenyl disulphide (See Baerlocher, F. J., Baerlocher, M. O., Langler, R. F., MacQuarrie, S. L., and Marchand, M. E., Aust J. Chem.—in press.) (1.0 g, 6.4 mmol) and hydrogen peroxide (30%, 1.5 g) were dissolved in glacial acetic acid (25 ml) and the reaction refluxed behind a safety shield for 0.5 h. Chloroform (100 ml) was added and the resultant mixture extracted with 2.5% sodium hydroxide solution (three—50 ml aliquots). The organic layer was dried (MgSO₄), filtered and the solvent evaporated. The crude product was chromatographed on silica gel (100 g) employing 1:1 chloroform/light petroleum (100 ml fractions) for elution. Fractions 11 and 12 were combined and concentrated affording clean phenyl methanethiosulfonate (4) (0.20 g, 1.1 mmol, 17%). Recrystallized (4) (methanol) had m.p. 88.9-90.4EC. l.r. 1335, 1145 cm¹. ¹H n.m.r. (270 MHz) δ3.19, s, 3H; 7.54, m, 3H; 7.72, d, 2H. ¹³C n.m.r. δ47.39, 127.93, 129.92, 131.67, 136.24. m/z 188 (35%, M⁺), 125 (57), 109 (100).

(B) Benzenesulfenyl Chloride From Disulphide, Then Reaction With Methanesulfinate Anions

Diphenyl disulphide (1.0 g, 4.6 mmol) was dissolved in dry methylene chloride (5 ml) and a solution of sulfuryl chloride (0.6 g, 4.6 mmol) in dry methylene chloride (5 ml) added dropwise. Upon completion of the addition, the reaction mixture was refluxed for 0.5 h.

A solution of sodium methanesulfinate (0.94 g, 9.2 mmol) in acetone (40 ml) and water (10 ml) was added to the reaction mixture which was then immersed in a constant temperature bath at 50EC for 1 h. The workup described for part (A) fumished diphenyl disulphide (0.35 g, 35% from column fractions 2 and 3) and the thiosulfonate (4) (0.66 g, 3.5 mmol, 38%, from fractions 7-17).

(C) Benzenesulfenyl Chloride From Mercaptan, Then Reaction With Methanesulfinate Anions

Benzenethiol (1.0 g, 9.0 mmol) was reacted with sulfuryl chloride (1.3 g, 9.7 mmol) and sodium methanesulfinate (0.96 g, 9.4 mmol) as described for diphenyl disulphide in part (B). Extractive workup and column chromatography, as described in part (B), fumished diphenyl disulphide (0. 11 g, from fraction 3) and the thiosulfonate (4)(0.95 g, 5.0 mmol, 55%, from fractions 7-11).

Preparation of Methyl Ethanethiosulfonate (5)

(A) Sodium benzenethiolate (2.1 g, 15.6 mmol) was dissolved in acetone (50 ml) and ethanesulfonyl chloride (1.0 g, 7.8 mmol) added. The reaction mixture was refluxed for 1 h. Chloroform (200 ml) was added and the resultant mixture washed with water (100 ml). The aqueous layer was concentrated and dried in vacuuo for 8 h, producing a mixture (1.12 g) of sodium ethanesulfinate and sodium chloride. The product had ¹H n.m.r. (270 MHz, D₂O+DSS) δ1.08, t, 3H; 2.33, q, 2H. ¹³C n.m.r. δ7.90, 56.44.

(B) Dimethyl disulphide (0.45 g, 4.8 mmol) was dissolved in dry methyiene chloride (5 ml) and a solution of sulfuryl chloride (0.65 g, 4.8 mmol) in dry methylene chloride (5 ml) added dropwise. Upon completion of the addition, the reaction mixture was refluxed for 0.5 h. A solution of the mixture of sodium ethanesulfinate and sodium chloride (1.12 g) in water (10 ml) and acetone (40 ml) was added. The reaction mixture was immersed in a constant temperature bath at 50EC for 1 h.

Chloroform (200 ml) was added and the resultant mixture extracted with water (100 ml). The organic layer was dried (MgSO₄), filtered and the solvent evaporated. The crude product was chromatographed on silica gel (100 g) employing 1:1 chloroform/light petroleum (100 ml fractions) for elution. Fractions 7-9 were concentrated and combined affording oily methyl ethanethiosulfonate (5)(0.32 g, 2.3 mmol, 29%). l.r. 1325, 1140 cm⁻¹. ¹H n.m.r. (270 MHz) δ1.48, t, 3H; 2.66, s, 3H; 3.34, q, 2H. ¹³C n.m.r. δ8.37, 18.21, 55.61. m/z 140 (75%, M⁺), 61 (47),48 (100).

Preparation of p-Nitrophenyl Methanethiosulfonate (6)

p-Nitrophenyl methyl disulphide (See Baerlocher, F. J., Baerlocher, M. O., Langler, R. F., MacQuarrie, S. L., and Marchand, M. E., Aust J. Chem.—in press.) (1.0 g, 5.0 mmol) was dissolved in dry methylene chloride (5 ml) and a solution of sulfuryl chloride (0.67 g, 5.0 mmol) in dry methylene chloride (5 ml) added dropwise. The reaction mixture was refluxed for 0.5 h. solution of sodium methanesulfinate (0.5 g, 5.0 mmol) in acetone (40 ml) and water (10 ml) was added and the reaction mixture immersed in a constant temperature bath at 50EC for 1 h.

Chloroform (200 ml) was added and the resultant mixture washed with water (100 ml). The organic layer was dried (MgSO₄), filtered and concentrated. The crude was recrystallized from methanol (8 ml) and the first crop chromatographed on silica gel (50 g) employing chloroform (50 ml fractions) for elution. Fraction 4 was concentrated affording clean p-nitrophenyl methanethiosulfonate (6) (0.31 g, 1.3 mmol, 26%). The nitrothiosulfonate (6) had m.p. 98-99EC. (Found: C, 36.1; H, 3.0. C₇H₇NO₄S₂ requires C, 36.0; H, 3.0%). l.r. 1530, 1440, 1145 cm⁻¹. ¹H n.m.r. (270 MHz) δ3.27, s, 3H; 7.92, d, 2H; 8.33, d, 2H. ¹³C n.m.r. δ48.56, 124.63, 135.40, 136.73, 149.56. m/z 233 (79%, M⁺), 170 (100).

Preparation of p-Nitrophenyl p-Toluenethiosulfonate (8)

p-Nitrophenyl methyl disufphide (7) (See Baerlocher, F. J., Baerlocher, M. O., Langler, R. F., MacQuarrie, S. L., and Marchand, M. E., Aust. J. Chem.—in press.) (2.5 g, 12.4 mmol) was converted into p-nitrophenyl p-toluenethiosulfonate (8) using the procedure (replace sodium methanesulfinate with sodium p-toluenesulfinate) outlined above for the preparation of (6). Crude product was not recrystallized but was chromatographed on silica gel (150 g) employing 1:1 chloroform/light petroleum (100 ml fractions) for elution. Fractions 3-10 were combined and concentrated and the product recrystallized (methanol). Recrystallized (8) was sublimed (110OEC/2 Torr/12 h) affording p-nitrophenyl p-toluenethiosulfonate (8)(1.4 g, 4.6 mmol, 37%). The thiosulfonate (8) had m.p. 135-137EC. (Found: C, 49.6; H, 3.4. C₁₃H₁₁NO₄S₂ requires C, 50.5; H, 3.6%). l.r. 1525, 1345, 1145 cm⁻¹.¹H n.m.r. (270 MHz) δ2.44, s, 3H; 7.25, d, 2H, 7.50, d, 2H; 7.60, d, 2H; 8.18, d, 2H. ¹³C n.m.r. δ21.73, 124.15, 127.57, 129.76, 135.77, 137.10, 140.17, 145.60, 149.38.

Preparation of o-Carbomethoxyphenyl Methanethiosulfonate (18)

o-Mercapto methylbenzoate (See Baerlocher, F. J., Baerlocher, M. O., Langler, R. F., MacQuarrie, S. L., and Marchand, M. E., Aust. J. Chem.—in press.) (2.0 g, 11.9 mmol) was dissolved in dry methylene chloride (5 ml) and a solution of sulfuryl chloride (1.6 g, 11.9 mmol) in dry methylene chloride (5 ml) added dropwise. The reaction mixture was refluxed for 0.5 h. A solution of sodium methanesulfinate (1.2 g, 11.9 mmol) in acetone (40 ml) and water (10 ml) was added to the reaction mixture which was immersed in a constant temperature bath at 50EC for 1 h.

Chloroform (200 ml) was added and the resultant mixture washed with water (100 ml). The organic layer was dried (MgSO₄), filtered and the solvent evaporated. Crude product was chromatographed on silica gel (100 g) employing 1:1 chloroform/light petroleum (100 ml fractions) for elution. Fractions 8-18 were combined and concentrated affording thiosulfonate (18) (2.0 g, 8.1 mmol, 68%). After recrystallization from methanol, o-carbomethoxyphenyl methanethiosulfonate (18) had m.p. 37.9-38.4EC. (Found: C, 44.4; H, 4.1. C₉H₁₀O₄S₂ requires C, 43.9; H, 4.1%). l.r. 1730, 1335, 1140 cm⁻¹. ¹H n.m.r. (270 MHz) δ3.24, s, 3H; 3.95, s, 3H; 7.61, m, 2H; 7.91, m, 2H. ¹³C n.m.r. δ48.73, 52.79, 127.54, 130.60, 131.25, 132.35, 135.73, 138.34, 168.83. m/z 167 (100%, M⁺—CH₃SO₂).

Preparation of Carbomethoxymethyl p-Toluenethiosulfonate (9)

A solution of p-nitrophenyl p-toluenethiosulfonate (8) (1.0 g, 3.2 mmol) in dimethyl sulfoxide (5 ml) was added to a solution of sodium methylthioglycollate (0.4 g, 3.2 mmol) in dimethyl sulfoxide (5 ml) and the reaction mixture stirred at ambient temperature for 2.5 h.

2.5% Hydrochloric acid (200 ml) was added and the resultant mixture extracted with diethyl ether (three—100 ml aliquots). The organic layers were combined and concentrated and the extractive procedure repeated. The combined organic layers were dried (MgSO₄), filtered and the solvent evaporated. Crude product was chromatographed on silica gel (100 g) employing chloroform (100 ml fractions) for elution. Fractions 6 and 7 were combined and concentrated, yielding oily thiosulfonate (9). l.r. 1745, 1320, 1150 cm⁻¹. ¹H n.m.r. (270 MHz) δ2.47, s, 3H; 3.72, s, 3H; 4.11, s, 2H; 7.38, d, 2H; 7.82, d, 2H. ¹³C n.m.r. δ21.73, 53.08, 60.93, 128.54, 129.90, 135.71, 145.52, 162.99. m/z 228 (3%, M⁺—CH₅O), 155 (51), 91 (100).

Administration—Pharmaceutical Compositions

For use as a medicine, the compound of the present invention may be administered to an animal including human being either as it is or in the form of a pharmaceutical composition containing, for example, 0.01-99.5%, preferably 0.5-90%, of the compound in a pharmaceutically acceptable nontoxic, inert carrier.

As the carrier, one or more of solid, semisolid, or liquid diluent, filler, and other formulation auxiliaries may be employed. The pharmaceutical composition is preferably administered in unit dosage forms. The pharmaceutical composition of the present invention may be administered orally, parenterally (e.g. intravenously), locally (e.g. transdermally), or rectally. Of course, dosage forms suited for respective routes of administration should be selected.

Oral administration may be carried out using solid or liquid unit dosage forms such as bulk powders, powders, tablets, dragees, capsules, granules, suspensions, solutions, syrups, drops, sublingual tablets, etc.

Bulk powders may be manufactured by comminuting the active substance into a finely divided form. Powders may be manufactured by comminuting the active substance into a finely-divided form and blending it with a similarly comminuted pharmaceutical carrier, e.g. an edible carbohydrate such as starch or mannitol. Where necessary, a corrigent, a preservative, a dispersant, a coloring agent, a perfume, etc. may also be added.

Capsules may be manufactured by filling said finely-divided bulk powders or powders, or granules described below for tablets, in capsule shells such as gelatin capsule shells. Preceding the filling operation, a lubricant or a fluidizing agent, such as colloidal silica, talc, magnesium stearate, calcium stearate or solid polyethylene glycol, may be blended with the powders. Improvement in the efficacy of the drug after ingestion may be expected when a disintegrator or a solubilizer, such as carboxymethylcellulose, carboxymethylcellulose calcium, low-substitution-degree hydroxypropylcellulose, roscarmellose sodium, carboxymethylstarch sodium, calcium carbonate or sodium carbonate, is added.

Soft capsules may be provided by suspending said finely divided powders in vegetable oil, polyethylene glycol, glycerin, or a surfactant and wrapping the suspension in gelatin sheets. Tablets may be manufactured by adding an excipient to said powders, granulating or slugging the mixture, adding a disintegrator and/or a lubricant, and compressing the whole composition. A powdery mixture may be prepared by mixing said finely divided powders with said diluent or a base. Where necessary, a binder (e.g. carboxymethylcellulose sodium, methylcellulose, hydroxypropylmethylcellulose, gelatin, polyvinylpyrrolidone, polyvinyl alcohol, etc.), a dissolution retardant (e.g. paraffin), a reabsorption agent (e.g. quaternary salts), and an adsorbent (e.g. bentonite, kaolin, dicalcium phosphate, etc.) may be added. The powdery mixture may be processed into granules by wetting it with a binder, e.g. a syrup, a starch paste, gum arabic, a solution of cellulose, or a solution of a high polymer, stirring to mix, drying it, and pulverizing the same. Instead of granulating such powders, it is possible to compress the powders with a tablet machine and crush the resulting slugs of crude form to prepare granules. The resulting granules may be protected against interadhesion by the addition of a lubricant such as stearic acid, a salt of stearic acid, talc or mineral oil. The mixture thus lubricated is then compressed. The resulting uncoated tablets may be coated with a film coating composition or a sugar coating composition.

The compound of the invention may be mixed with a free-flowing inert carrier and the mixture be directly compressed without resort to the above-mentioned granulation or slugging process. A transparent or translucent protective coat consisted of, for example, a hermetic shellac coat, a sugar or polymer coat, or a polishing wax coat may also be applied. Other oral compositions such as a solution, a syrup, and an elixir may also be provided in unit dosage forms each containing a predetermined amount of the drug substance. Syrups may be manufactured by dissolving the compound in suitable flavored aqueous media, while elixirs may be manufactured using nontoxic alcoholic vehicles. Suspensions may be formulated by dispersing the compound in nontoxic vehicles. Where necessary, solubilizers and emulsifiers (e.g. ethoxylated isostearyl alcohol, polyoxyethylene sorbitol ester, etc.), preservatives, and flavorants (e.g. peppermint oil, saccharin, etc.) may also be added.

Where necessary, the unit dosage formulation for oral administration may be microencapsulated. This formulation may be coated or embedded in a polymer, wax or other matrix to provide a prolonged action or sustained release dosage form.

Parenteral administration may be carried out using liquid unit dosage forms for subcutaneous, intramuscular, or intravenous injection, e.g. solutions and suspensions. Such unit dosage forms may be manufactured by suspending or dissolving a predetermined amount of the compound of the invention in an injectable nontoxic liquid vehicle, for example an aqueous vehicle or an oily vehicle, and sterilizing the resulting suspension or solution. For isotonizing an injection, a nontoxic salt or salt solution may be added. Moreover, stabilizers, preservatives, emulsifiers, etc. may also be added.

Rectal administration may be carried out by using suppositories manufactured by dissolving or suspending the compound in a low-melting water-soluble or waterinsoluble solid carrier such as polyethylene glycol, caccao butter, semisynthetic oil (e g. Witepsol®), a higher ester (e.g. myristyl palmitate) or a mixture thereof.

The invention may be varied in any number of ways as would be apparent to a person skilled in the art and all obvious equivalents and the like are meant to fall within the scope of this description and claims. The description is meant to serve as a guide to interpret the claims and not to limit them unnecessarily.

1. Sulfone disulphides of the general formula RSO₂CH₂SSR¹, wherein R is phenyl or lower alkyl, and R¹ is lower alkyl or phenyl.

2. Sulfone disulphides as claimed in claim 1 selected from the group consisting of
C₆H₅SO₂CH₂SSCH₃,
CH₃SO₂CH₂SSCH₃, and
CH₃SO₂CH₂SSC₆H₅.

3. A process for preparing a sulfone disulphide of the general formula as claimed in claim 1, which comprises reacting a transition metal oxidant or a peroxyanhydride oxidant with a symmetrical dialkyl or arylalkyl disulphide to obtain an α-ester disulphide compound of the formula RSSCH₂OC(O)R¹, wherein R and R¹ are as defined in claim 1, which compound is further reacted with a sulfinic acid salt to obtain the required compound.

4. Compounds of the general formula RSSCH₂OC(O)R¹, wherein R and R¹ may be the same or different and each is selected from the group of substituents comprising lower alkyl or phenyl.

5. Compounds of the general formula as set out in claim 4 wherein lower alkyl is methyl or ethyl.

6. 2,3-Dithiabutyl benzoate.

7. 1-Phenyl-1,2-dithiapropyl propionate.

8. Methyl 3,4-dithia-5-propionoxypentanoate.

9. 2,3-Dithiabutane-1,4-dipropionate.

10. 1-p-Toluenesulfonyl4-propionoxy-2,3-dithiabutane.

11. Phenacyl methyl disulphide.

12. A process for preparing a compound of the general formula as claimed in claim 4, which comprises reacting a transition metal oxidant or a peroxyanhydride oxidant with a symmetrical dialkyl or arylalkyl disulfide to obtain the α-ester disulfide compound.

13. A process for preparing compounds of the formula RSO₂CH₂SSR¹, wherein R and R¹ may be the same or different and each is selected from lower alkyl and phenyl, which comprises reacting a transition metal oxidant or a peroxyanhydride oxidant with a symmetrical dialkyl or arylalkyl disulfide to obtain the α-ester disulfide compound RSSCH₂OC(O)R¹, wherein R and R¹ are as defined above, which compound is further reacted with a sulfinic acid salt to obtain the required compound.

14. A process for preparing a compound of the formula PhSO₂CH₂SSCH₂CH₃, wherein Ph is phenyl which comprises reacting a transition metal oxidant with dimethyl disulfide to obtain an α-ester disulfide compound of the formula H₃SSCH₂OC(O)CH₂CH₃, which compound is further reacted with the sodium salt of p-toluenesulfinic acid in either aqueous acetonitrile or aqueous acetone to obtain the title compound or the compound is further reacted with potassium p-toluenethiosulfonate to yield the title compound.

15. A process for the preparation of α-ester disulphides and α-sulfonyl disuiphides of the formula RSSCH₂SO₂R¹, wherein R and R¹ may be the same or different and each is selected from the group comprising lower alkyl, phenyl, phenyl substituted with ????, which comprises reacting a compound of the formula RSH, wherein R is as defined above with a compound of the formula ClSCH₂OC(O)CH₂CH₃, in the presence of a base to yield a compound of the formula RSSCH₂OC(O)CH₂CH₃, wherein R is as defined above; reacting this compound with a compound of the formula R¹SO₂Na, wherein R¹ is as defined above, to obtain the desired compound.

16. A compound of the formula ClSCH₂OC(O)CH₂CH₃.

17. Antifungal agents comprising as active ingredient at least one antifungally active compound of the formulae RSO₂CH₂SSR¹, wherein R is lower alkyl or phenyl and R¹ is lower alkyl or phenyl, and RC(O)OCH₂SSR¹, wherein R and R¹ are as defined above together with a pharmaceutically acceptable carrier.

18. Antifungal agents comprising as active ingredients a therapeutically effective amount of at least one compound of the formula 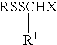
wherein R¹ is H or CH₃;
R is CH₃, CH₂CH₃, C₆H₅, o-CH_{3O}₂C(C₆H₄), o-CH₃SO₂(C₆H₄, p-CH₃SO₂(C₆H₄), O NO₂(C₆H₄), m-NO₂(C₆H₄), p-NO₂(C₆H₄), or CH₂O₂CCH₂CH₃ ; and
X is SO₂(C₆H₄)CH₃-p, SO₂CH₃, SO₂C₆H₅, SO₂CH₂CH₃, H, O₂CCH₃CH₃, or CO₂CH₃;
and a pharmaceutically acceptable carrier.

19. Antifungal agents as claimed in claim 17 wherein the active ingredient is selected from the group of compounds consisting of
o-CH₃C(O)(C₆H₄)SSCH₃,
[o-CH₃SO₂(C₆H₄)S]₂,
[p-CH₃SO₂(C₆H₄)S]₂,
m-O₂N(C₆H₄)SSCH₃,
o-O₂N(C₆H₄)SSCH₃,
p-O₂N(C₆H₄)SSCH₃,
p-CH₃(C₆H₄)SO₂CH₂SSCH₃,
C₆H₅SO₂CH₂SSCH3,
CH₃SO₂CH₂SSCH₃,
CH₃CH₂C(O)OCH₂SSCH₃,
CH₃SO₂CH₂SSPC₆H₅,
CH₃SO₂CH₂SSCH₂CH₃,
CH₃SSCH₂OC(O)CH₃,
CH₃SSCH₂OC(O)CH₂CH₃,
CH₃SSCH₂OC(O)PC₆H₅, and
PhSSCH₂OC(O)CH₂CH₃,
together with a pharmaceutically acceptable carrier.

20. An antiproliferative agent active against mammalian cells comprising as active ingredient a therapeutically effective amount of at least one antiproliferative active compound of the formulae
RSO₂CH₂SSR¹ (I)
wherein R is lower alkyl or phenyl and R¹ is lower alkyl or phenyl, and
RC(O)OCH₂SSR¹ (II)
wherein R and R¹ are as defined above, together with a pharmaceutically acceptable carrier.

21. An antiproliferative agent active against mammalian cells comprising a therapeutically effective amount of at least one compound selected from the group of compounds defined by the formula RSSCH₂X, wherein X is SO₂(C₆H₄)CH₃-p, SO₂CH₃, CO₂CH₃, O₂CCH₃, SCH₃, O₂CCH₂CH₃, (O)CC₆H₅ or CH₂SO₂CH₃; and R is CH₃ or C₆H₅ together with a pharmaceutically acceptable carrier.

22. An antiproliferative agent active against mammalian cells comprising as active ingredient a therapeutically effective amount of at least one antiproliferative compound selected from the group of compounds consisting of
CH₃(C₆H₄)SO₂CH₂SSCH₃
(C₆H₅)SS(C₆H₅)
CH₃SO₂CH₂SS(C₆H₅)
CH₃SSCH₂OC(O)CH₃
CH₃SO₂CH₂SSCH₂CH₂CH₂CH₂CH₃
CH₃SO₂S(C₆H₅)
o-, m- and p-NO₂(C₆H₄)SSCH₃
CH₃SCH₂SSCH₃
CH₃OC(O)CH₂SSCH₂C(O)OCH₃
(C₆H₅)C(O)CH₂SSCH₃
(C₆H₅)SSCH₂OC(O)CH₂CH₃
CH₃SO₂CH₂CH₂SSCH₃, and
CH₃SSCH₂C(O)OCH3, together with a pharmaceutically acceptable carrier.

23. An anti-tumour agent active against leukemic cells comprising as active ingredient a therapeutically effective amount of a compound of the formula CH₃SO₂CH₂CH₂SSCH₃ and a pharmaceutically acceptable carrier.

24. An antiproliferative agent active against mammalian cells comprising as active ingredient a therapeutically effective amount of at least one therapeutically effective amount of CH₃SCH₂SSCH₃ together with a pharmaceutically acceptable carrier.

25. An antiproliferative agent active against mammalian cells comprising as active ingredient a therapeutically effective amount of at least one therapeutically effective amount of (C₆H₅)C(O)CH₂SSCH₃ together with a pharmaceutically acceptable carrier.

26. An antiproliferative agent active against mammalian cells comprising as active ingredient a therapeutically effective amount of at least one therapeutically effective amount of (C₆H₅)SSCH₂OC(O)CH₂CH₃ together with a pharmaceutically acceptable carrier.

27. An antifungal agent comprising as active ingredient a therapeutically effective amount of a compound of the formula (C₆H₅)SSCH₂OC(O)CH₂CH₃ together with a pharmaceutical carrier.

28. The use of at least one of the compounds as claimed in any of claims 1 to 8 and 27 in the preparation of a medicament for antifungal treatment.

29. The use of at least one of the compounds as set out in any of claims 21 to 26 in the preparation of a medicament for the treatment of cancer.
